(19) United States
(12) Patent Application Publication
(10) Pub. No.: US 2026/0216350 A1
(43) Pub. Date: Jul. 30, 2026

(54) INSULIN COMPLEXES

(71) Applicant: UNIVERSITY OF NOTRE DAME DU LAC, South Bend, IN (US)

(72) Inventors: Matthew J. Webber, South Bend, IN (US); Yuanhui Xiang, South Bend, IN (US); Sijie Xian, South Bend, IN (US)

(21) Appl. No.: 19/146,172

(22) PCT Filed: Jan. 10, 2024
(86) PCT No.: PCT/US2024/011034

§ 371 Date: Jul. 7, 2025

Related U.S. Application Data
(60) Provisional application No. 63/479,387, filed on Jan. 11, 2023.

(51) Int. Cl. A61K 47/59; A61K 38/28; A61K 47/54; A61P 3/10

(52) U.S. Cl. CPC A61K 47/595; A61K 38/28; A61K 47/54; A61P 3/10

(57) ABSTRACT

Described are complexes comprising insulin. The complexes described herein comprise insulin or a macromolecule attached to a glucose-binding diboronate motif (B1), and a macromolecule or insulin attached to a diol (D1). Further disclosed are methods of correcting blood glucose levels in a subject in need thereof, the methods comprising administering a complex described herein or a pharmaceutical composition comprising a complex described herein to the subject in need thereof.

### CROSS-REFERENCE TO RELATED APPLICATION(S)

This application claims the benefit of and priority to U.S. Provisional Patent Application No. 63/479,387, filed on Jan. 11, 2023, which is incorporated herein by reference in its entirety.

### TECHNICAL FIELD

The present disclosure relates to complexes comprising insulin. The complexes comprise insulin or a macromolecule attached to a glucose-binding diboronate motif (B¹), and a macromolecule or insulin attached to a diol (D¹). Further disclosed are methods of correcting blood glucose levels in a subject in need thereof, the methods comprising administering a complex described herein or a pharmaceutical composition comprising a complex described herein to the subject in need thereof.

### INTRODUCTION

Diabetes is among the most pressing global healthcare challenges. The incidence of all forms of diabetes is increasing, coupled with extensive suffering, comorbidity, and economic burden. The absence or dysfunctional signaling of insulin, a hormone secreted by the pancreas in response to elevated blood glucose levels that triggers glucose uptake and storage, is central to the pathology of diabetes. Exogenous insulin is critical to manage type 1 diabetes and is often also used in later stages of type 2 diabetes. Insulin is typically self-administered, with best outcomes following a strict treatment regimen. Some individuals respond well to insulin therapy, yet many still suffer complications that arise from poor adherence or from inadequate glycemic control. An overdose of insulin may lead to acute hypoglycemia, with serious and even lethal outcomes. For this reason, insulin is often underdosed to avoid the acute risks from overdose. Unfortunately, prolonged blood glucose instability and hyperglycemia that arises from under-dosing insulin leads to its own chronic comorbidities, including cardiovascular and neurological diseases, retinopathy, nephropathy, and non-healing wounds. Diabetics also have an increased rate of total, cardiovascular, and cancer mortality. There is thus a growing need to improve blood glucose control and avoid the acute and chronic health complications that arise from blood glucose instability.

Progress in diabetes treatment has been realized by insulin variants with tunable pharmacokinetics, as well as advances in pumps, continuous glucose monitors, and related hardware. Key developments in recent decades include the approval of insulin variants with either rapid-acting (prandial) and long-lasting (basal) function. Current clinically used basal variants offer ~1 day of blood glucose support (e.g., Insulin Glargine and Insulin Detemir), though nascent week-long variants fused to antibody Fc domains (Insulin Efsitora Alfa) or that bind with high affinity to circulating albumin (Insulin Icodec) are on the cusp of clinical use. While day- and weeklong variants offer basal support with enhanced duration and ease of use, these do not adjust bioavailability or potency as a function of blood glucose level.

Glucose-responsive therapy remains a goal in the development of materials and formulations for insulin delivery. Glucose-responsive therapy involves varying dosage according to real-time disease state (i.e., glucose level), delivering on a vision of a synthetic “closed-loop” therapy that senses changes in blood glucose and responds by tuning the bioavailability and/or potency of insulin. Glucose sensing is typically achieved by integrating one of three mechanisms into materials design: (i) enzyme-catalyzed pH change, (ii) glucose binding proteins, or (iii) glucose-binding synthetic motifs. However, recreating the natural dynamics of glycemic control, with both peaks and troughs, is still a challenge in the materials-based approaches explored thus far. Moreover, since managing diabetes often requires life-long therapy, glucose-responsive technologies must be amenable to serial self-administration with practical dosing schedules. As such, and despite decades of progress, there is so far limited demonstration of systems that tune insulin bioavailability/potency according to real-time need to meet both basal and prandial insulin requirements in a single platform.

What is needed are improved compositions and methods for insulin delivery. More specifically, approaches that are sensitive to real-time therapeutic need dictated by blood glucose level, while also offering convenient dosing schedules, are absent in the current standard of care.

### SUMMARY

In some aspects, the present disclosure provides complex comprising insulin attached to B¹ (insulin-B¹) and a macromolecule attached to D¹ (macromolecule-D¹); or insulin attached to D¹ (insulin-D¹) and a macromolecule attached to B¹ (macromolecule-B¹);
wherein:
B¹ is

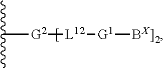

wherein:
B^{X}, at each occurrence, is B(OH)₂ or [B(OH)₃]⁻;
G¹, at each occurrence, is independently a pyridylene or a phenylene, wherein G¹ is optionally substituted with 1-2 substituents independently selected from the group consisting of halogen, —CN, C₁₋₄alkyl, —NO₂, C₃₋₄cycloalkyl, C₁₋₂haloalkyl, —OC₁₋₄alkyl, —OC₃₋₄cycloalkyl, and —OC₁₋₂haloalkyl, wherein each cycloalkyl is optionally substituted with 1-4 substituents independently selected from the group consisting of halogen, C₁₋₄alkyl, and C₁₋₂haloalkyl;
L¹², at each occurrence, is independently a C₁₋₆alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with a first substituent selected from the group consisting of halogen, cyano, C₁₋₆alkyl, C₁₋₄haloalkyl, oxo, —OR^{2x}, —N(R^{2x})₂, —SR^{2x}, —SO₂R^{2x}, —C(O)R^{2x}, —C(O)OR^{2x}, —C(O)N(R^{2x})₂, —C₁₋₆alkylene-OR^{2x}, —C₁₋₆alkylene-SR^{2x}, —C₁₋₆alkylene-N(R^{2x})₂, —C₁₋₆alkylene-SO₂R^{2x}, —C₁₋₆alkylene-C(O)R^{2x}, —C₁₋₆alkylene-C(O)OR^{2x}, and —C₁₋₆alkylene-C(O)N(R^{2x})₂, and optionally further substituted with 1-3 substituents independently selected from the group consisting of halogen, cyano, C₁₋₄alkyl, C₁₋₄haloalkyl, and —OR^{2x};
R^{2x}, at each occurrence, is hydrogen, C₃₋₄cycloalkyl, C₁₋₂haloalkyl, —OC₁₋₄alkyl, —OC₃₋₄cycloalkyl, or —OC₁₋₂haloalkyl, wherein each cycloalkyl is optionally substituted with 1-4 substituents independently selected from the group consisting of halogen, C₁₋₄alkyl, and C₁₋₂haloalkyl; and
D¹ is a diol moiety comprising

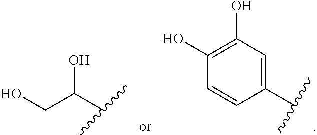 [SMILES: *C(O)CO ; *c1ccc(O)c(O)c1]

The complex may comprise the insulin-B¹ and the macromolecule-D¹. The molar ratio of the insulin-B¹ to the macromolecule-D¹ may be from 1:1 to 110:1.
G² may be phenylene

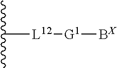

may be:

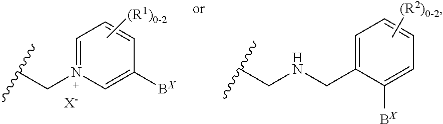

wherein:
R¹, at each occurrence, is independently halogen, —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl;
R², at each occurrence, is independently —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl; and
X⁻ is an anion having a net charge of −1.

X⁻ may be Br⁻, Cl⁻, NO₃⁻, H₂PO₄⁻, H₂PO₃⁻, HSO₄⁻, HSO₃⁻, H₃C—SO₃, HCO₃⁻, HCO₂⁻, H₃C—CO₂⁻, HC₂O₄⁻, or TsO⁻. In some instances, X⁻ is Br⁻ or Cl⁻.

B¹ may be attached to the insulin by a linking moiety.

The linking moiety may comprise

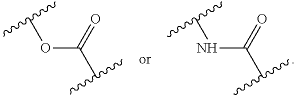 [SMILES: *OC(*)=O ; *NC(*)=O]

The linking moiety may comprise:

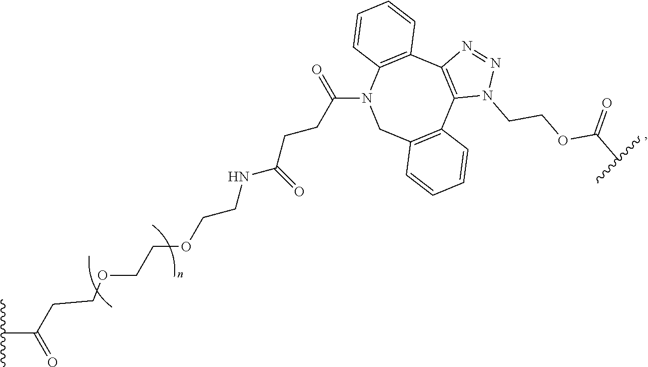

wherein n is 1 to 20.

B¹ may be attached to the insulin's Lys_{B29} residue.

D may be:

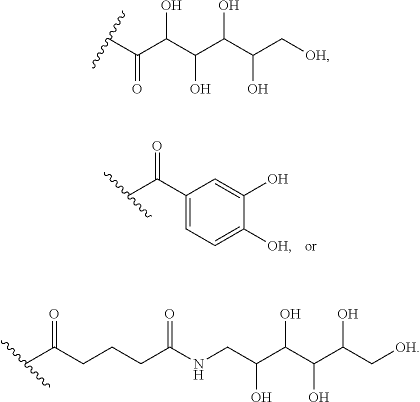 [SMILES: *C(=O)C(O)C(O)C(O)C(O)CO ; *C(=O)c1ccc(O)c(O)c1 ; *C(=O)CCCC(=O)NCC(O)C(O)C(O)C(O)CO]

The macromolecule may be a dendrimer. The dendrimer may be a polyamidoamine dendrimer, a polyethylenimine dendrimer, a polyester dendrimer, or a lysine dendrimer. The dendrimer may be a 16-arm to 256-arm dendrimer.

In other aspects, the present disclosure provides pharmaceutical compositions comprising a complex described herein and a pharmaceutically acceptable excipient.

In other aspects, the present disclosure provides methods of correcting blood glucose levels in a subject in need thereof, the method comprising: administering a complex or a pharmaceutical composition described herein, to a subject in need thereof. The subject in need thereof may have diabetes. The insulin-B¹ may be administered to the subject at 0.05 mg/kg to 10 mg/kg. In some instances, following administration of the complex or pharmaceutical composition, the subject may have blood glucose levels of 40 mg/dL to 800 mg/dL. In some instances, following administration of the complex or pharmaceutical composition, the subject may have corrected blood glucose levels for at least 1 day.

### BRIEF DESCRIPTION OF THE DRAWINGS

FIG. 1A shows the chemical structures of insulin site-specifically modified at the B29 lysine with a glucose-binding diboronate motif (“Insulin-DiPBA”), and a generation 6 (G6) PAMAM Dendrimer-Diol modified on its periphery with glucose-like diol molecules (“Dendrimer-Diol”).

FIG. 1B schematically illustrates the electrostatic interactions between the net-negative Insulin-DiPBA and the positive Dendrimer-Diol, as well as the DiPBA-diol dynamic-covalent bonding, that combine to form a nanocomplex.

FIG. 2A shows the ¹H NMR characterization of DiPBA-N₃.

FIG. 2B shows the ¹H NMR characterization of DiPyr-N₃.

FIG. 3A shows the mass spectrometry (MS) spectrum of dibenzocyclooctyne (DBCO)-modified insulin (“I_{DBCO}”).

FIG. 3B schematically illustrates the disulfide reduction and trypsin digest locations in I_{DBCO}.

FIG. 3C shows the liquid chromatography (LC) chromatograms for the different digestion fragments of I_{DBCO}.

FIG. 3D shows the mass spectrometry (MS) fragment analysis for the digestion fragments of I_{DBCO}.

FIG. 4A shows the liquid chromatography chromatograms of B29-specific modification with DiPBA using copper-free click chemistry.

FIG. 4B shows the mass spectrometry spectra of B29-specific modification with DiPBA using copper-free click chemistry.

FIG. 5 shows the dose-response curves for the in vitro assay in C2C12 cells measured as pAKT versus total AKT.

FIG. 6 shows the potency of Insulin-DiPBA in overnight-fasted STZ diabetic mice compared to native (rH Insulin) at 0.1 mg/kg.

FIG. 7A graphically shows the binding affinity of insulin at insulin receptor A (IR-A).

FIG. 7B graphically shows the binding affinity of insulin at insulin receptor B (IR-B).

FIG. 7C graphically shows the binding affinity of insulin at insulin-like growth factor-1 receptor (IGF-1R).

FIG. 8A graphically shows the binding affinity of B29-modified Insulin-Dibenzocyclooctyne (DBCO) (Ins-DBCO, i.e., I_{DBCO}) at insulin receptor A (IR-A).

FIG. 8B graphically shows the binding affinity of B29-modified Insulin-DBCO (Ins-DBCO, i.e., I_{DBCO}) at insulin receptor B (IR-B).

FIG. 8C graphically shows the binding affinity of B29-modified Insulin-DBCO (Ins-DBCO, i.e., I_{DBCO}) at insulin-like growth factor-1 receptor (IGF-1R).

FIG. 9A graphically shows the binding affinity of B29-modified Insulin-DiPBA (Ins-DiPBA, i.e., I_{DiPBA}) at insulin receptor A (IR-A).

FIG. 9B graphically shows the binding affinity of B29-modified Insulin-DiPBA (Ins-DiPBA, i.e., I_{DiPBA}) at insulin receptor B (IR-B).

FIG. 9C graphically shows the binding affinity of B29-modified Insulin-DiPBA (Ins-DiPBA, i.e., I_{DiPBA}) at insulin-like growth factor-1 receptor (IGF-1R).

FIG. 10A-10C show ¹H NMR characterization spectra for exemplary Dendrimer-Diols.

FIG. 10A shows the ¹H NMR spectrum for Dendrimer-Diol (G2).

FIG. 10B shows the ¹H NMR spectrum for Dendrimer-Diol (G4).

FIG. 10C shows the ¹H NMR spectrum for Dendrimer-Diol (G6).

FIG. 11 graphically shows the percent cell viability measured by an in vitro cytotoxicity assay exposing C2C12 cells to Insulin-DiPBA and Dendrimer-Diol (G6).

FIG. 12 shows the isothermal titration calorimetry results for Insulin-DiPBA binding to a model glucono-δ-lactone (GdL)-derived diol small molecule at pH 7.4.

FIG. 13 shows photographs demonstrating that the mixture of Insulin-DiPBA and Dendrimer-Diol is soluble and translucent at pH 5, but precipitates under neutral conditions.

FIG. 14A graphically shows the isothermal titration calorimetry results for Insulin-DiPBA binding to a model GdL-derived diol small molecule at pH 5.0.

FIG. 14B graphically shows the isothermal titration calorimetry results for Insulin-DiPBA binding to a model GdL-derived diol small molecule at pH 3.5.

FIG. 15 is a bar graph showing the turbidity of the 1:1 mixture, Insulin-DiPBA, and Dendrimer-Diol at the concentrations used for injection with in vivo studies in mice (n=3/group).

FIG. 16 is a bar graph showing that tuning the ratio of positive charge, originating from the Dendrimer_{Diol}, to negative charge from the Insulin-DiPBA enables precipitation to be optimized, as measured by sample turbidity (n=3/ratio/group, mean±SD shown).

FIG. 17A is a bar graph showing the encapsulation efficiency of Dendrimer_{Diol} and Insulin-DiPBA in 1:1 nanocomplexes.

FIG. 17B shows the analytical HPLC calibration curve obtained by measuring the residual soluble Dendrimer_{Diol} following centrifugation of the nanocomplexes.

FIG. 18A schematically illustrates the Dendrimer-Diol and modified variant Dendrimer-Diol(COOH).

FIG. 18B is a bar graph showing zeta potentials of unaltered Dendrimer-Diol and modified variant Dendrimer-Diol(COOH).

FIG. 18C is a bar graph showing the relative turbidity (%) of Dendrimer-Diol complexed in a 1:1 molar ratio with Insulin-DiPBA and Dendrimer-Diol(COOH) complexed in a 1:1 molar ratio with Insulin-DiPBA.

FIG. 19A is the dynamic light scattering (DLS) spectrum for recombinant Human (rH) insulin.

FIG. 19B is the dynamic light scattering (DLS) spectrum for Insulin-DiPBA.

FIG. 20 is a bar graph showing the turbidity intensity of complexes between different diol-modified dendrimer generations with Insulin-DiPBA at different charge ratios.

FIG. 21 graphically shows the zeta potentials of Dendrimer-Diol and Insulin-DiPBA individually, as well as that for the 1:1 charge ratio mixture (complex). Zeta potentials were measured over a pH range of 2-12 to estimate isoelectric points (dashed vertical lines) where zeta potential is 0 (n=3/titrations/group, mean±SD shown).

FIG. 22 is a negative-stained transmission electron microscopy image showing the resulting nanocomplexes from the 1:1 charge ratio mixture of Insulin-DiPBA and Dendrimer-Diol.

FIG. 23 shows the relative turbidity of the 1:1 charge ratio mixture of Insulin-DiPBA and Dendrimer-Diol prepared at different glucose concentrations (n=3/group, mean±SD shown).

FIG. 24 graphically shows release of free Insulin-DiPBA from pre-formed complex in buffer conditions of 0 mg/dL, 100 mg/dL, and 400 mg/dL glucose (n=3/group, mean±SD shown).

FIG. 25 graphically shows release of free Insulin-DiPBA from pre-formed complexes in buffer conditions of 100 mg/dL glucose without or with 10% FBS (n=3/group).

FIG. 26 graphically shows cycling the nanocomplexes between conditions of no glucose (PBS, gray) and high glucose buffer (400 mg/dL, white), measuring cumulative insulin release as well as the release rate between each sampling point (n=3/group, mean±SD shown).

FIG. 27A graphically shows the release of insulin from 1.5:1 complexes in low vs. high glucose conditions.

FIG. 27B graphically shows the cyclic release of insulin from 1.5:1 complex when alternating between low and high glucose levels over time.

FIG. 28 shows the circular dichroism (CD) spectra of released Insulin-DiPBA compared to fresh samples of Insulin-DiPBA and recombinant human insulin.

FIG. 29 graphically shows the blood glucose levels (BGL) during the treatment of overnight-fasted STZ diabetic mice with carrier control (Dendrimer-Diol) or the Insulin-Dendrimer nanocomplex formulations at a charge ratio of 1:1 or 1.5:1. Following administration of treatment (t=0) three consecutive intraperitoneal glucose tolerance tests (IP-GTT) were performed while maintaining mice in a fasted state (n=6 for carrier and n=8 for treatment groups, mean±SEM shown).

FIGS. 30A-30C graphically show the area under the curve (AUC) after treatment with an Insulin-Dendrimer nanocomplex formulation at a charge ratio of 1:1 or 1.5:1 (t=0) and an intraperitoneal glucose tolerance a test (GTT).

FIG. 30A is a bar graph showing the AUC after the first GTT.

FIG. 30B is a bar graph showing the AUC after the second GTT.

FIG. 30C is a bar graph showing the AUC in after the third GTT.

FIG. 31 graphically shows the blood glucose levels (BGL) of overnight-fasted STZ diabetic mice were administered Insulin-Dendrimer nanocomplex formulations at a charge ratio of 1:1 or 1.5:1 and compared to a potency-matched dose of Insulin Detemir. IPGTT was then performed at day 0 (D0). Insulin Detemir was serially dosed daily, and mice were subjected to follow-up intraperitoneal glucose tolerance test were preformed again after a brief 3-hour fast on D2 and D4 following treatment with Insulin-Dendrimer nanocomplex formulations, hyperglycemia was restored in all mice (n=10/group, mean±SEM shown).

FIG. 32A-32C graphically show the glucose tolerance test (GTT) area under the curve (AUC) quantified in the response to the 1:1 and 1.5:1 of the Insulin-Dendrimer nanocomplex formulations (n=10/group, mean±SEM shown, *-P<0.05 determined from Student's t-test).

FIG. 32A is a bar graph showing the glucose tolerance test (GTT) area under the curve (AUC) quantified in the response to the 1:1 and 1.5:1 of the Insulin-Dendrimer nanocomplex formulations at day 0 (D0).

FIG. 32B is a bar graph showing the glucose tolerance test (GTT) area under the curve (AUC) quantified in the response to the 1:1 and 1.5:1 of the Insulin-Dendrimer nanocomplex formulations at day 2 (D2).

FIG. 32C is a bar graph showing the glucose tolerance test (GTT) area under the curve (AUC) quantified in the response to the 1:1 and 1.5:1 of the Insulin-Dendrimer nanocomplex formulations at day 4 (D4).

FIG. 33 schematically illustrates the approach to measuring changes in serum insulin resulting in STZ diabetic mice upon glucose challenge at DO, D2, and D4 following treatment with the Insulin-Dendrimer nanocomplex formulated at a charge ratio of 1:1.

FIG. 34A-34C graphically show the serum insulin and blood glucose measured 30 minutes prior to (Pre) and 30 minutes following (Post) administering IPGTT. Studies performed in 3 cohorts of mice for each treatment (n=6/cohort), with each cohort used for one of the timepoints (n=6/group, mean±SEM shown, *-P<0.05 from a paired Student's t-test).

FIG. 34A is a bar graph showing the serum insulin and blood glucose of mice cohort 1.

FIG. 34B is a bar graph showing the serum insulin and blood glucose of mice cohort 2.

FIG. 34C is a bar graph showing the serum insulin and blood glucose of mice cohort 3.

FIG. 35A-35C graphically show the serum insulin concentrations for carrier-treated STZ mice before and after intraperitoneal glucose tolerance tests (IP-GTT).

FIG. 35A graphically shows the serum insulin concentrations for carrier treated STZ mice at day 0.

FIG. 35B graphically shows the serum insulin concentrations for carrier treated STZ mice at day 2.

FIG. 35C graphically shows the serum insulin concentrations for carrier treated STZ mice at day 4.

FIG. 36 graphically shows the kinetic profile of serum insulin levels and blood glucose in STZ diabetic mice for an IPGTT performed 48 hours after treatment with the Insulin-Dendrimer nanocomplex formulated at a charge ratio of 1:1 (solid lines) or a control of the Dendrimer-Diol carrier alone (STZ control, dashed lines). Study was performed in 5 cohorts of mice per group (n=5/cohort) with each cohort yielding two timepoints shown in the curve according to the table in the supporting information (n=5/group/timepoint, mean±SEM shown).

FIG. 37A-37D graphically show serial dosing of Insulin-Dendrimer nanocomplex formulations at a charge ratio of 1:1 or 1.5:1 administered every 5 days and compared to controls of mice treated every 5 days with the Dendrimer-Diol carrier or every day with a potency-matched dose of insulin detemir. Blood glucose levels were collected after a brief 3 h fast, with the yellow shaded region indicating the normal blood glucose range for a healthy mouse (n=10/group, mean±SEM shown).

FIG. 37A graphically shows blood glucose levels in STZ diabetic mice.

FIG. 37B graphically shows animal weights throughout the study and presented relative to the pre-STZ weight of each mouse.

FIG. 37C is a bar graph showing the serum chemistry in healthy mice, with values of each marker presented relative to those for untreated healthy mice (n=4/group, mean±SEM shown, ANOVA performed with Tukey post hoc test for each analyte).

FIG. 37D shows optical microscopy of stained liver and kidney sections for 1:1 and 1.5:1 nanocomplex formulations dosed serially for one month in health mice.

FIG. 38 shows hemolysis of Insulin-DiPBA and Dendrimer-Diol over a range of concentrations in excess of their maximum possible exposure in blood (n=3/group).

FIG. 39A-39D graphically show alloxan-treated diabetic swine (n=6) exposed to an oral glucose tolerance test (OGTT) administered at t=0, with all blood glucose levels normalized to fasted blood glucose levels for each individual untreated pig. The initial OGTT response for 2 days prior to treatment was measured for each individual pig, averaged, and plotted as the untreated control (black dashed trace). On DO, the Insulin-Dendrimer nanocomplex formulation at a charge ratio of 1:1 was first administered, and blood glucose was first monitored for 3 h prior to OGTT. OGTT was then performed on DO and each subsequent day (D1-D6) for a week following initial treatment with the Insulin-Dendrimer nanocomplex.

FIG. 39A graphically shows relative blood glucose levels.

FIG. 39B is a bar graph showing fasting blood glucose levels.

FIG. 39C is a bar graph showing final blood glucose level at 150 minutes after OGTT.

FIG. 39D is a bar graph showing the area under the curve (AUC) for each OGTT in the cohort of swine prior treatment (untreated) and OFTT performed during days 0-6.

FIG. 40A-40D graphically show the kinetics of serum insulin and blood glucose levels for diabetic swine during oral glucose tolerance tests where the Insulin-Dendrimer nanocomplex formulation at a charge ratio of 1:1 was first administered (“day 0”).

FIG. 40A graphically shows serum insulin and blood glucose levels the day before treatment with the Insulin-Dendrimer nanocomplex formulation (“day −1”).

FIG. 40B graphically shows serum insulin and blood glucose levels the day after treatment with the Insulin-Dendrimer nanocomplex formulation.

FIG. 40C graphically shows serum insulin and blood glucose levels 3 days after treatment with the Insulin-Dendrimer nanocomplex formulation.

FIG. 40D graphically shows serum insulin and blood glucose levels 5 days after treatment with the Insulin-Dendrimer nanocomplex formulation.

FIG. 41A graphically shows serum insulin concentrations for treated diabetic swine in with an oral glucose tolerance test (OGTT) following treatment with the nanocomplex formulation on day −1, day 1, day 3, and day 5.

FIG. 41B graphically shows blood glucose levels for treated diabetic swine in with an oral glucose tolerance test (OGTT) following treatment with the nanocomplex formulation on day −1, day 1, day 3, and day 5.

### DETAILED DESCRIPTIONS

Before any embodiments of the disclosure are explained in detail, it is to be understood that the disclosure is not limited in its application to the details of construction and the arrangement of components set forth in the following description or illustrated in the following drawings. The disclosure is capable of other embodiments and of being practiced or of being carried out in various way.

### I. Definitions

Unless otherwise defined, all technical and scientific terms used herein have the same meaning as commonly understood by one of ordinary skill in the art. In case of conflict, the present document, including definitions, will control, Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “may,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result may be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, “treatment” or “treating” refers to prophylaxis of, preventing, suppressing, repressing, reversing, alleviating, ameliorating, or inhibiting the progress of biological process including a disorder or disease, or eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkoxy,” as used herein, refers to a group —O-alkyl. Representative examples of alkoxy include, but are not limited to, methoxy, ethoxy, propoxy, 2-propoxy, butoxy and tert-butoxy.

The term “alkyl,” as used herein, means a straight or branched, saturated hydrocarbon chain. The term “lower alkyl” or “C₁₋₆alkyl” means a straight or branched chain hydrocarbon containing from 1 to 6 carbon atoms. The term “C₁₋₄alkyl” means a straight or branched chain hydrocarbon containing from 1 to 4 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkenyl,” as used herein, means a straight or branched, hydrocarbon chain containing at least one carbon-carbon double bond.

The term “alkoxyalkyl,” as used herein, refers to an alkoxy group, as defined herein, appended to the parent molecular moiety through an alkyl group, as defined herein.

The term “alkylamino,” as used herein, means at least one alkyl group, as defined herein, is appended to the parent molecular moiety through an amino group, as defined herein. The term “amide,” as used herein, means —C(O)NR— or —NRC(O)—, wherein R may be hydrogen, alkyl, cycloalkyl, aryl, heteroaryl, heterocycle, alkenyl, or heteroalkyl.

The term “aminoalkyl” as used herein, means at least one amino group, as defined herein, is appended to the parent molecular moiety through an alkylene group, as defined herein.

The term “amino,” as used herein, means —NRₓR_{y}, wherein Rₓ and R_{y} may be hydrogen, alkyl, cycloalkyl, aryl, heteroaryl, heterocycle, alkenyl, or heteroalkyl. In the case of an aminoalkyl group or any other moiety where amino appends together two other moieties, amino may be —NRₓ—, wherein Rₓ may be hydrogen, alkyl, cycloalkyl, aryl, heteroaryl, heterocycle, alkenyl, or heteroalkyl.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

The term “cyanoalkyl,” as used herein, means at least one —CN group, is appended to the parent molecular moiety through an alkylene group, as defined herein.

The term “cycloalkoxy,” as used herein, refers to a cycloalkyl group, as defined herein, appended to the parent molecular moiety through an oxygen atom.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The term “cycloalkenyl” or “cycloalkene,” as used herein, means a non-aromatic monocyclic or multicyclic ring system containing all carbon atoms as ring members and at least one carbon-carbon double bond and preferably having from 5-10 carbon atoms per ring. The term “cycloalkenyl” is used herein to refer to a cycloalkene when present as a substituent. A cycloalkenyl may be a monocyclic cycloalkenyl (e.g., cyclopentenyl), a fused bicyclic cycloalkenyl (e.g., octahydronaphthalenyl), or a bridged cycloalkenyl in which two non-adjacent atoms of a ring are linked by an alkylene bridge of 1, 2, 3, or 4 carbon atoms (e.g., bicyclo[2.2.1]heptenyl). Exemplary monocyclic cycloalkenyl rings include cyclopentenyl, cyclohexenyl or cycloheptenyl. Exemplary monocyclic cycloalkenyl rings include cyclopentenyl, cyclohexenyl or cycloheptenyl.

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms cycloalkylene and heterocyclylene refer to divalent groups derived from the base ring, i.e., cycloalkane, heterocycle. For purposes of illustration, examples of cycloalkylene and heterocyclylene include, respectively,

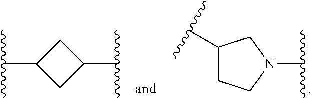 [SMILES: *C1CC(*)C1 ; *C1CCN(*)C1]

Cycloalkylene and heterocyclylene include a geminal divalent groups such as 1,1-C₃₋₆cycloalkylene

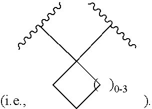

A further example is 1,1-cyclopropylene

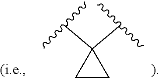 [SMILES: *C1(*)CC1]

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “haloalkoxy,” as used herein, means at least one haloalkyl group, as defined herein, is appended to the parent molecular moiety through an oxygen atom.

The term “halocycloalkyl,” as used herein, means a cycloalkyl group, as defined herein, in which one or more hydrogen atoms are replaced by a halogen.

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g., 1, 2, 3, or 4 heteroatoms independently selected from O, S, and N). The five membered aromatic monocyclic rings have two double bonds, and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxomayyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, 3-oxaspiro[5.5]undemayyl, 6-oxaspiro[2.5]octan-1-yl, and 3-oxabicyclo[3.1.0]hexan-6-yl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]demaye), and oxa-adamantane (2-oxatricyclo[3.3.1.13,7]demaye). The monocyclic, bicyclic, and tricyclic heterocyclyls are connected to the parent molecular moiety at a non-aromatic ring atom.

The term “hydroxyl” or “hydroxy,” as used herein, means an —OH group.

The term “hydroxyalkyl,” as used herein, means at least one —OH group, is appended to the parent molecular moiety through an alkylene group, as defined herein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

### II. Compositions

### A. Complexes

Exemplary complexes described herein comprise insulin or a macromolecule attached to a glucose-binding diboronate motif (B¹), and a macromolecule or insulin attached to a diol (D¹).

The term “complex,” as used herein, means a substance formed by the union of simpler substances (as molecules, compounds, or ions) and held together by force. The complexes described herein are held together by dynamic-covalent bonds, namely, the dynamic covalent bonds between the glucose-binding diboronate motif (B¹) and diol (D¹). As used herein the term “dynamic-covalent bond” refers to a covalent bond that may reversibly form and dissociate and is typically equilibrium-governed. Further, the constitution of such dynamic systems may respond to changes in chemical environment (e.g., complexing entities) or physical conditions (e.g., temperature, mechanical stress, electric field, irradiation) to alter their extent of bond formation.

The glucose-binding diboronate motif (B¹) may be any suitable moiety comprising two boronic acid/boronate moieties (“B^{X}”). Depending on the pH of the complex's environment and the specific boronic acid's pKa, each boronic acid moiety may exist in the complex in its trigonal planar form, “B(OH)₂,” or its tetrahedral boronate form, “[B(OH)₃]⁻,” as illustrated below.

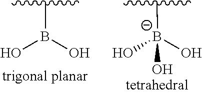

Accordingly, B^{X} may be B(OH)₂ or [B(OH)₃]⁻.

The diol (D¹) may be any suitable moiety comprising a vicinal diol (two hydroxyl groups attached to adjacent atoms).

Complexes of the present disclosure may comprise:
insulin attached to B¹ (insulin-B¹) and a macromolecule attached to D¹ (macromolecule-D¹); or
insulin attached to D¹ (insulin-D¹) and a macromolecule attached to B¹ (macromolecule-B¹);
wherein:
B¹ is

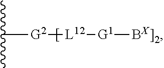

wherein:
B^{X}, at each occurrence, is B(OH)₂ or [B(OH)₃]—;
G¹, at each occurrence, is independently a pyridylene or a phenylene, wherein G1 is optionally substituted with 1-2 substituents independently selected from the group consisting of halogen, —CN, C₁₋₄alkyl, —NO₂, C₃₋₄cycloalkyl, C₁₋₂haloalkyl, —OC₁₋₄alkyl, —OC₃₋₄cycloalkyl, and —OC₁₋₂haloalkyl, wherein each cycloalkyl is optionally substituted with 1-4 substituents independently selected from the group consisting of halogen, C₁₋₄alkyl, and C₁₋₂haloalkyl;
L¹², at each occurrence, is independently a C₁₋₆alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with a first substituent selected from the group consisting of halogen, cyano, C₁₋₆alkyl, C₁₋₄haloalkyl, oxo, —OR^{2x}, —N(R^{2x})₂, —SR^{2x}, —SO₂R^{2x}, —C(O)R^{2x}, —C(O)OR^{2x}, —C(O)N(R^{2x})₂, —C₁₋₆alkylene-OR^{2x}, —C₁₋₆alkylene-SR^{2x}, —C₁₋₆alkylene-N(R^{2x})₂, —C₁₋₆alkylene-SO₂R^{2x}, —C₁₋₆alkylene-C(O)R^{2x}, —C₁₋₆alkylene-C(O)OR^{2x}, and —C₁₋₆alkylene-C(O)N(R^{2x})₂, and optionally further substituted with 1-3 substituents independently selected from the group consisting of halogen, cyano, C₁₋₄alkyl, C₁₋₄haloalkyl, and —OR^{2x};
R^{2x}, at each occurrence, is hydrogen, C₃₋₄cycloalkyl, C₁₋₂haloalkyl, —OC₁₋₄alkyl, —OC₃₋₄cycloalkyl, or —OC₁₋₂haloalkyl, wherein each cycloalkyl is optionally substituted with 1-4 substituents independently selected from the group consisting of halogen, C₁₋₄alkyl, and C₁₋₂haloalkyl; and
D¹ is a diol moiety comprising or

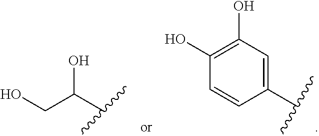 [SMILES: *C(O)CO ; *c1ccc(O)c(O)c1]

In some instances, G² is phenylene.

In some instances,

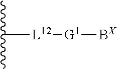

is:

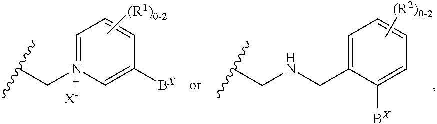

wherein:
R¹, at each occurrence, is independently halogen, —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl;
R², at each occurrence, is independently —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl; and
X⁻ is an anion having a net charge of −1.

In some instances, X⁻ is Br⁻, Cl⁻, NO₃⁻, H₂PO₄⁻, H₂PO₃⁻, HSO₄⁻, HSO₃⁻, H₃C—SO₃⁻, HCO₃⁻, HCO₂⁻, H₃C—CO₂⁻, HC₂O₄⁻, or TsO⁻.

In some instances, X⁻ is Br⁻ or Cl⁻.

In some instances, B¹ is attached to the insulin by a linking moiety.

In some instances, the linking moiety comprises:

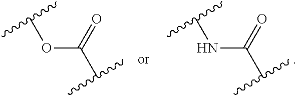 [SMILES: *OC(*)=O ; *NC(*)=O]

In some instances, the linking moiety comprises:

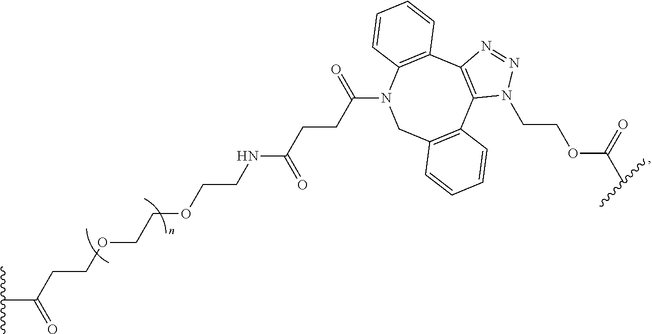

wherein n is 1 to 20.

In some instances, B¹ is attached to the insulin's Lys_{B29} residue.

In some instances, D¹ is:

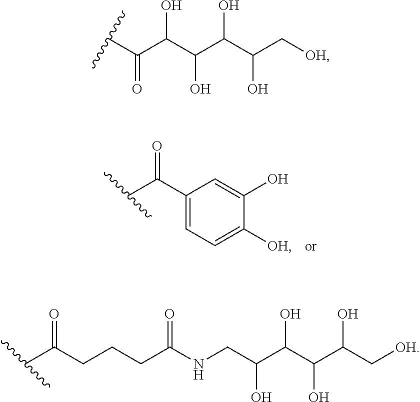 [SMILES: *C(=O)C(O)C(O)C(O)C(O)CO ; *C(=O)c1ccc(O)c(O)c1 ; *C(=O)CCCC(=O)NCC(O)C(O)C(O)C(O)CO]

In some instances, the macromolecule is a dendrimer. The term “dendrimer,” as used herein means a highly ordered and branched polymer emanating from a central core. The dendrimer may be a polyamidoamine dendrimer, a polyethylenimine dendrimer, polyester dendrimer, or a lysine dendrimer. In some instances, dendrimer is a 16-arm to 256-arm dendrimer.

In some instances, the complex comprises the insulin-B¹ and the macromolecule-D¹. In various instances, the molar ratio of the insulin-B¹ to the macromolecule-D¹ may be from 1:1 to 110:1. In some instances, the molar ratio of the insulin-B¹ to the macromolecule-D¹ may be from 5:1 to 105:1; 10:1 to 100:1; 15:1 to 95:1; 20:1 to 90:1; 25:1 to 85:1; 30:1 to 80:1; 35:1 to 75:1; 40:1 to 70:1; 45:1 to 65:1; or 50:1 to 60:1. In some instances, the molar ratio of the insulin-B¹ to the macromolecule-D¹ may be no greater than 110:1; no greater than 100:1; no greater than 90:1; no greater than 80:1; no greater than 70:1; no greater than 60:1; no greater than 50:1; no greater than 40:1; no greater than 30:1; no greater than 20:1; no greater than 10:1; or no greater than 5:1. In some instances, the molar ratio of the insulin-B¹ to the macromolecule-D¹ may be no less than 1:1; no less than 5:1; no less than 10:1; no less than 20:1; no less than 30:1; no less than 40:1; no less than 50:1; no less than 60:1; no less than 70:1; no less than 80:1; no less than 90:1; no less than 100:1; or no less than 105:1.

### B. General Syntheses

Exemplary insulin and macromolecules attached to B¹ and D¹ (insulin-B¹, macromolecule-B¹, insulin-D¹, and macromolecule-D¹) may be prepared according to the general schemes below.

Abbreviations which have been used in the Schemes that follow are:
TBAI is tetra-n-butylammonium iodide;
DMF is dimethyl formamide;
TEA is triethylamine;
ACN is acetonitrile;
Bi(OTf)₃ is bismuth (III) trifluoromethanesulfonate;
THF is tetrahydrofuran; and
Pd/C is palladium on carbon.

General Scheme 1, below, illustrates a general method for preparing exemplary B¹-substituted azide intermediates of formula E.

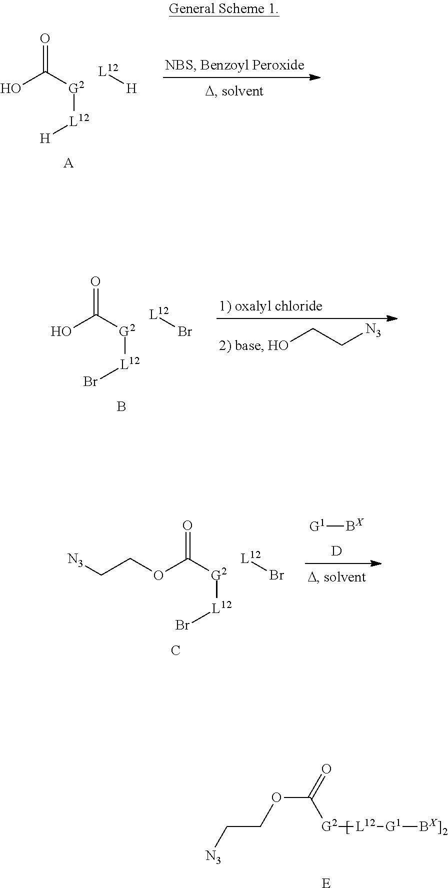

As shown in General Scheme 1 above, in some instances, B¹-substituted azide intermediates of formula E may be prepared by subjecting a compound of formula A to suitable bromination conditions (e.g., NBS and benzoyl peroxide in chloroform) to provide a brominated intermediate of formula B. The intermediate of formula B may be transformed into an acyl chloride under suitable conditions (e.g., oxalyl chloride), followed by exposure to a suitable base and a suitable azide (e.g., TEA and

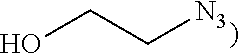

to provide an intermediate compound of formula C. An intermediate compound of formula C may then be reacted with an intermediate of formula D under suitable conditions to provide exemplary azide intermediates of formula E.

General Scheme 2, below, illustrates a general method for preparing exemplary B¹-substituted acid intermediates of formula F.

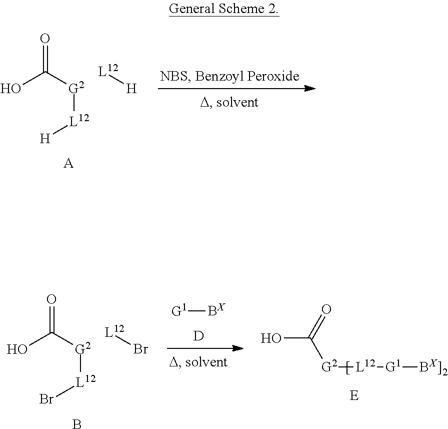

As shown in General Scheme 2 above, in some instances, B¹-substituted acid intermediates of formula F may be prepared by subjecting a compound of formula A to suitable bromination conditions (e.g., NBS and benzoyl peroxide in chloroform) to provide a brominated intermediate of formula B. An intermediate compound of formula B may then be reacted with an intermediate of formula D under suitable conditions to provide exemplary B¹-substituted acid intermediates of formula F.

The B¹-substituted intermediates, e.g., an exemplary azide intermediate of formula E or an exemplary acid intermediate of formula F, may be carried forward to prepare various exemplary insulin-B¹ or macromolecule-B¹. For example, General Scheme 3, below, illustrates an exemplary method for preparing insulin and macromolecules attached to B¹ (insulin-B¹ and macromolecule-B¹) using an exemplary azide intermediate of formula E.

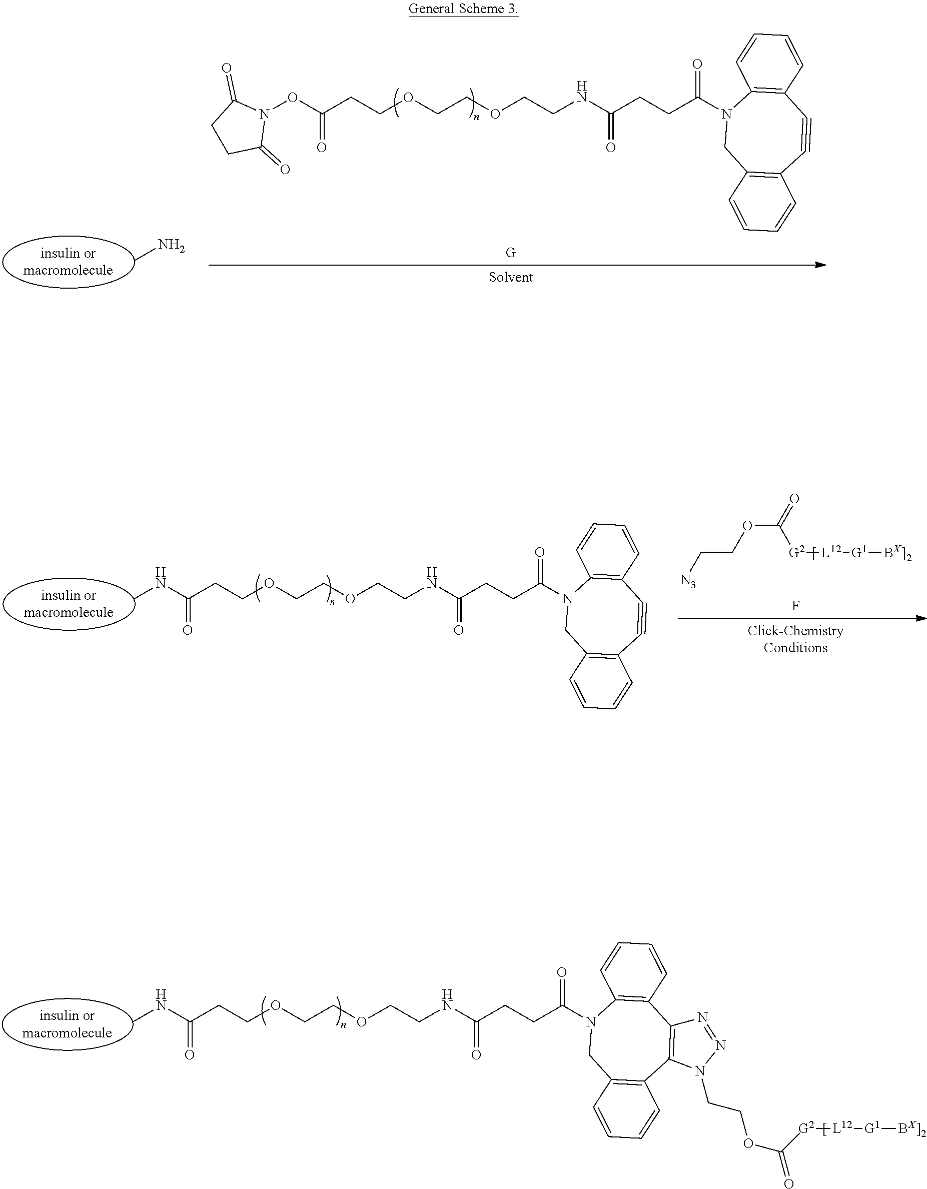

As shown in General Scheme 3 above, in some instances, an exemplary insulin-B¹ or macromolecule-B¹ may be prepared by reacting insulin or a macromolecule comprising a —NH₂ group with a compound of formula G under suitable conditions to provide an intermediate of formula H. Intermediates of formula H may be reacted with an azide intermediate of formula E under suitable click-chemistry conditions to provide an exemplary insulin-B¹ or macromolecule-B¹.

General Scheme 4, below, illustrates an alternative exemplary method for preparing insulin and macromolecules attached to B¹ (insulin-B¹ and macromolecule-B¹) using an exemplary acid intermediate of formula F.

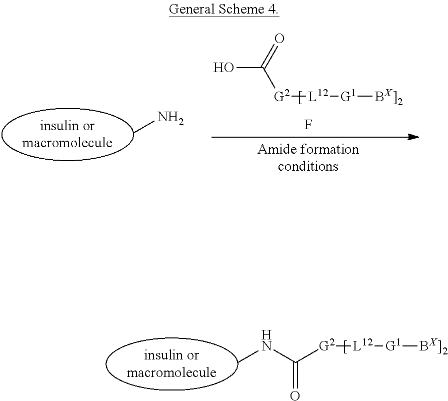

As shown in General Scheme 4 above, in some instances, an exemplary insulin-B¹ or macromolecule-B¹ may be prepared by reacting insulin or a macromolecule comprising a —NH₂ group with an acid intermediate of formula F to provide an exemplary insulin-B¹ or macromolecule-B¹.

General Scheme 5, below, illustrates an exemplary method for preparing insulin and macromolecules attached to D¹ (insulin-D¹ and macromolecule-D¹).

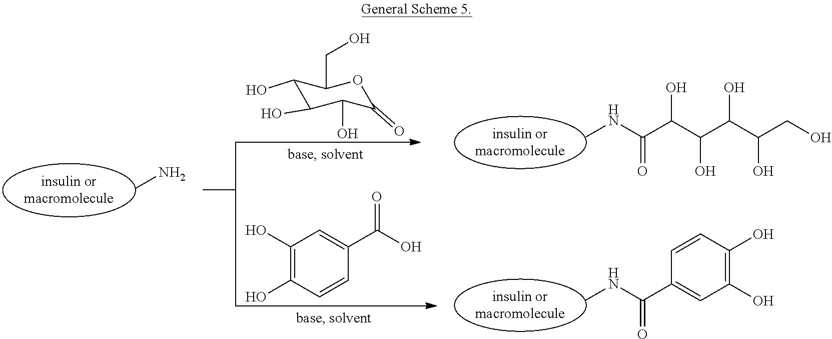

As shown in General Scheme 5 above, an exemplary insulin-D¹ or macromolecule-D¹ may be prepared by reacting insulin or a macromolecule comprising a —NH₂ group with a diol-containing compound (e.g., glucono-δ-lactone (GdL) or 3,4-dihydroxybenzoic acid)under suitable coupling conditions (e.g., in presence of TEA and MeOH).

The compounds and intermediates may be isolated and purified by methods well-known to those skilled in the art of organic synthesis. Examples of conventional methods for isolating and purifying compounds may include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5th edition (1989), by Furniss, Hannaford, Smith, and Tatchell, pub. Longman Scientific & Technical, Essex CM20 2JE, England.

A disclosed compound may have at least one basic nitrogen whereby the compound may be treated with an acid to form a desired salt. For example, a compound may be reacted with an acid at or above room temperature to provide the desired salt, which is deposited, and collected by filtration after cooling. Examples of acids suitable for the reaction may include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step may vary depending on the reactants employed and substituents present in the reactants used. Specific procedures are provided in the Examples section. Reactions may be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or may be prepared by one skilled in the art from commercially available materials using methods described in the chemical literature. Starting materials, if not commercially available, may be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which may be found in P G M Wuts and T W Greene, in Greene's book titled Protective Groups in Organic Synthesis (4^{th} ed.), John Wiley & Sons, NY (2006), which is incorporated herein by reference in its entirety. Synthesis of the compounds of the invention may be accomplished by methods analogous to those described in the synthetic schemes described hereinabove and in specific examples.

When an optically active form of a disclosed compound is required, it may be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it may be obtained by carrying out one of the above procedures using a pure geometric isomer as a starting material, or by resolution of a mixture of the geometric isomers of the compound or intermediates using a standard procedure such as chromatographic separation.

It may be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### C. Pharmaceutical Compositions

In various instances, a complex described herein may be included in a pharmaceutical composition. The complexes may be incorporated into pharmaceutical compositions suitable for administration to a subject (such as a patient, which may be a human or non-human). The pharmaceutical compositions may include a “therapeutically effective amount” or a “prophylactically effective amount” of the active agent (insulin or analogues or variants thereof).

A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A “therapeutically effective amount” is also one in which any toxic or detrimental effects are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

The pharmaceutical compositions may include pharmaceutically acceptable excipients. The term “pharmaceutically acceptable excipient,” as used herein, means a non-toxic, inert solid, semi-solid or liquid filler, diluent, encapsulating material, or formulation auxiliary of any type. Some examples of materials which may serve as pharmaceutically acceptable excipients are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants may also be present in the composition, according to the judgment of the formulator.

The disclosed compositions may be topically administered. Topical compositions, such as a topical composition comprising a disclosed complex and a pharmaceutically acceptable excipient, may be applied locally to the skin. The pharmaceutically acceptable excipient of the topical composition may aid penetration of the complexes into the skin. The pharmaceutically acceptable excipient may further include one or more optional components.

The amount of the pharmaceutically acceptable excipient employed in conjunction with the complex is sufficient to provide a practical quantity of composition for administration per unit dose of the medicament. Techniques and compositions for making dosage forms useful in the methods of this invention are described in the following references: Modern Pharmaceutics, Chapters 9 and 10, Banker & Rhodes, eds. (1979); Lieberman et al., Pharmaceutical Dosage Forms: Tablets (1981); and Ansel, Introduction to Pharmaceutical Dosage Forms, 2^{nd} ed., (1976).

A pharmaceutically acceptable excipient may include a single ingredient or a combination of two or more ingredients. For example, in topical compositions, the pharmaceutically acceptable excipient includes a topical excipient. Suitable topical excipients include one or more ingredients selected from phosphate buffered saline, isotonic water, deionized water, monofunctional alcohols, symmetrical alcohols, aloe vera gel, allantoin, glycerin, vitamin A and E oils, mineral oil, propylene glycol, PPG-2 myristyl propionate, dimethyl isosorbide, castor oil, combinations thereof, and the like. More particularly, excipients for skin applications include propylene glycol, dimethyl isosorbide, and water, and even more particularly, phosphate buffered saline, isotonic water, deionized water, monofunctional alcohols, and symmetrical alcohols.

The pharmaceutically acceptable excipient of a topical composition may further include one or more ingredients selected from emollients, propellants, solvents, humectants, thickeners, powders, fragrances, pigments, and preservatives, all of which are optional.

Suitable emollients include stearyl alcohol, glyceryl monoricinoleate, glyceryl monostearate, propane-1,2-diol, butane-1,3-diol, mink oil, cetyl alcohol, isopropyl isostearate, stearic acid, isobutyl palmitate, isocetyl stearate, oleyl alcohol, isopropyl laurate, hexyl laurate, decyl oleate, octademay-2-ol, isocetyl alcohol, cetyl palmitate, di-n-butyl sebacate, isopropyl myristate, isopropyl palmitate, isopropyl stearate, butyl stearate, polyethylene glycol, triethylene glycol, lanolin, sesame oil, coconut oil, arachis oil, castor oil, acetylated lanolin alcohols, petroleum, mineral oil, butyl myristate, isostearic acid, palmitic acid, isopropyl linoleate, lauryl lactate, myristyl lactate, decyl oleate, myristyl myristate, and combinations thereof. Specific emollients for skin include stearyl alcohol and polydimethylsiloxane. The amount of emollient(s) in a skin-based topical composition is typically about 5% to about 95%.

Suitable propellants include propane, butane, isobutane, dimethyl ether, carbon dioxide, nitrous oxide, and combinations thereof. The amount of propellant(s) in a topical composition is typically about 0% to about 95%.

Suitable solvents include water, ethyl alcohol, methylene chloride, isopropanol, castor oil, ethylene glycol monoethyl ether, diethylene glycol monobutyl ether, diethylene glycol monoethyl ether, dimethylsulfoxide, dimethyl formamide, tetrahydrofuran, and combinations thereof. Specific solvents include ethyl alcohol and homotopic alcohols. The amount of solvent(s) in a topical composition is typically about 0% to about 95%.

Suitable humectants include glycerin, sorbitol, sodium 2-pyrrolidone-5-carboxylate, soluble collagen, dibutyl phthalate, gelatin, and combinations thereof. Specific humectants include glycerin. The amount of humectant(s) in a topical composition is typically 0% to 95%. The amount of thickener(s) in a topical composition is typically about 0% to about 95%. Suitable powders include beta-cyclodextrins, hydroxypropyl cyclodextrins, chalk, talc, fullers earth, kaolin, starch, gums, colloidal silicon dioxide, sodium polyacrylate, tetra alkyl ammonium smectites, trialkyl aryl ammonium smectites, chemically-modified magnesium aluminum silicate, organically-modified Montmorillonite clay, hydrated aluminum silicate, fumed silica, carboxyvinyl polymer, sodium carboxymethyl cellulose, ethylene glycol monostearate, and combinations thereof. The amount of powder(s) in a topical composition is typically 0% to 95%. The amount of fragrance in a topical composition is typically about 0% to about 0.5%, particularly, about 0.001% to about 0.1%. Suitable pH adjusting additives include HCl or NaOH in amounts sufficient to adjust the pH of a topical pharmaceutical composition.

### III. Methods of Treatment

Further disclosed herein are methods of correcting glucose levels in a subject in need thereof. Exemplary method of correcting blood glucose levels in a subject in need thereof, comprise administering a complex described herein or a pharmaceutical composition comprising a complex described herein to a subject in need thereof.

In various instances, the insulin-B¹ may be administered to the subject at 0.05 mg/kg to 10 mg/kg. In some instances, the insulin-B¹ may be administered to the subject at 0.1 mg/kg to 10 mg/kg; 0.5 mg/kg to 9.5 mg/kg; 1 mg/kg to 9 mg/kg; 1.5 mg/kg to 8.5 mg/kg; 2 mg/kg to 8 mg/kg; 2.5 mg/kg to 7.5 mg/kg; 3 mg/kg to 7 mg/kg; 3.5 mg/kg to 6.5 mg/kg; 4 mg/kg to 6 mg/kg; 4.5 mg/kg to 5.5 mg/kg. In some instances, the insulin-B¹ may be administered to the subject at no greater than 10 mg/kg; no greater than 9 mg/kg; no greater than 8 mg/kg; no greater than 7 mg/kg; no greater than 6 mg/kg; no greater than 5 mg/kg; no greater than 4 mg/kg; no greater than 3 mg/kg; or no greater than 2 mg/kg. In some instances, the insulin-B¹ may be administered to the subject at no less than 0.05 mg/kg; no less than 0.1 mg/kg; no less than 0.5 mg/kg; no less than 1 mg/kg; no less than 2 mg/kg; no less than 3 mg/kg; no less than 4 mg/kg; no less than 5 mg/kg; no less than 6 mg/kg; no less than 7 mg/kg; or no less than 8 mg/kg.

In various instances, following administration of the complex or pharmaceutical composition, the subject may have blood glucose levels of 40 mg/dL to 800 mg/dL. In some instances, the subject may have blood glucose levels of 50 mg/dL to 700 mg/dL; 60 mg/dL to 600 mg/dL; 70 mg/dL to 500 mg/dL; 80 mg/dL to 450 mg/dL; 90 mg/dL to 400 mg/dL; 100 mg/dL to 350 mg/dL; 110 mg/dL to 300 mg/dL; 120 mg/dL to 150 mg/dL; or 130 mg/dL to 140 mg/dL. In various instances, following administration of the complex or pharmaceutical composition, the subject may have blood glucose levels of no greater than 800 mg/dL; no greater than 700 mg/dL; no greater than 600 mg/dL; no greater than 500 mg/dL; no greater than 400 mg/dL; no greater than 300 mg/dL; no greater than 200 mg/dL; no greater than 100 mg/dL; no greater than 90 mg/dL; no greater than 80 mg/dL; no greater than 70 mg/dL; no greater than 60 mg/dL; or no greater than 50 mg/dL. In some instances, the subject may have blood glucose levels of no less than 40 mg/dL; no less than 50 mg/dL; no less than 60 mg/dL; no less than 70 mg/dL; no less than 80 mg/dL; no less than 90 mg/dL; no less than 100 mg/dL; no less than 200 mg/dL; no less than 300 mg/dL; no less than 400 mg/dL; no less than 500 mg/dL; or no less than 600 mg/dL.

In various instances, following administration of the complex or pharmaceutical composition, the subject may have corrected blood glucose levels for at least 1 day. In various instances, following administration of the complex or pharmaceutical composition, the subject may have corrected blood glucose levels for at least 2 days; at least 3 days; at least 4 days; at least 5 days; at least 6 days; or at least 7 days.

The subject in need thereof may have a metabolic disease or disorder. The term “metabolic disorder” refers to any disorder that involves an alteration in the normal metabolism of carbohydrates, lipids, proteins, nucleic acids, or a combination thereof. A metabolic disorder is associated with either a deficiency or excess in a metabolic pathway resulting in an imbalance in metabolism of nucleic acids, proteins, lipids, and/or carbohydrates. Factors affecting metabolism include, and are not limited to, the endocrine (hormonal) control system (e.g., the insulin pathway, the enteroendocrine hormones including GLP-1, PYY or the like), the neural control system (e.g., GLP-1 in the brain), or the like. Examples of metabolic disorders include, but are not limited to, diabetes (e.g., Type I diabetes, Type II diabetes, gestational diabetes), hyperglycemia, hyperinsulinemia, insulin resistance, and obesity. In various instances, the subject in need thereof may have diabetes.

### IV. Examples

### A. General Materials and Methods

In Vitro Bioactivity Assays. C2C12 mouse myoblast cell line was purchased from ATCC and cultured with standard growth media consisting of Dulbecco's Modified Eagle's Medium (DMEM) with 4.5 g/L D-glucose, L-glutamine, and 110 mg/L sodium pyruvate supplemented with 10% fetal bovine serum (FBS) and 2% penicillin-streptomycin. C2C12 cells were incubated at 37° C. and 5% CO₂. To assess receptor activation, C2C12 cells were seeded in clear, flat-bottom 96-well tissue culture plates at a density of 25,000 cells/well with 200 μL standard growth media. After incubating for 24 h, the media was aspirated, and cells were washed twice with 200 μL of complete growth media followed by starving in 100 μL of unsupplemented DMEM overnight. Media was then aspirated from each well, and the cells were stimulated with 100 μL of insulin diluted in unsupplemented DMEM to desired concentrations (1E-1, 1E-2, 1E-3, 1E-4, 1E-5, 1E-6, 1E-7, and 1E-8 mg/mL, in triplicate) for 30 min while incubating at 37° C. Cells were then washed twice with 100 μL of cold 1× Tris-buffered saline (TBS), followed by the addition of 100 μL of lysis buffer into each well. Cells were lysed for 20 min at room temperature under shaking (350 rpm). Total AKT and phosphorylated AKT (pSer473) levels were quantified with PerkinElmer AlphaLISA SureFire Ultra AKT 1/2/3 (pS473) Assay Kit (500 Assay Points), and PerkinElmer Total AKT 1/2/3 AlphaLISA SureFire Ultra Detection Kit (500 Assay Points). Cell lysate (10 μL) was transferred to a PerkinElmer 384-well white optiPlate and assays were completed according to the manufacturer's protocol. Plates were incubated at room temperature for 20 h and AlphaLISA signal was analyzed using a BioTek Cytation 5 Cell Imaging Multimode Reader. The EC₅₀ value of each insulin derivative was then quantified by plotting the log of insulin concentration versus the pAKT to total AKT ratio.

Receptor Affinity Assays. Binding affinities of Insulin-DBCO, Insulin-DiPBA, and human insulin for human IR-A and IR-B were determined by competition with [¹²⁵I]-monoiodotyrosyl-TyrA14-insulin for IR-A or IR-B in cell membranes of mouse embryonic fibroblasts derived from IGF-1R knock-out mice and transfected to express the human IR-A or IR-B isoform at a high density of ~10⁵ receptors/cell. The cells were a kind gift of Prof Antonino Belfiore (Catanzaro, Italy). Radiolabeled [¹²⁵I]-monoiodotyrosyl-TyrA14-insulin was prepared from human insulin via radioiodination of TyrA14 according to a procedure previously described in detail. The cells were grown at 37° C. in a humid atmosphere (5% CO₂) in 87.6% DMEM containing glucose (4.5 g/L), 10% fetal bovine serum, L-glutamine (2 mmol/L), penicillin (100 U/mL) streptomycin (100 μg(mL), and puromycin (3 μg/mL). For the assay, the cells (about 38,000 per well) were washed twice with the binding buffer (100 mmol/L HEPES pH 7.6, 100 mmol/L NaCl, 5 mmol/L KCl, 1.3 mmol/L MgSO₄, 1 mmol/L EDTA, 10 mmol/L glucose, 15 mmol/L sodium acetate and 1% bovine serum albumin). The cells were incubated and stirred with increasing concentrations of insulin analogue and human [¹²⁵I]-monoiodotyrosyl-TyrA14-insulin (2200 Ci/mmol, 43,000 cpm, 0.043 nM) for 16 h at 5° C. in the binding buffer (total volume 250 mL). After incubation, the cells were washed twice with cold binding buffer and solubilized with 0.1 mol/L NaOH. The solutions of solubilized cells were counted for cell-associated radioactivity. The binding curve of each analogue was determined in duplicate points and the final dissociation constant (K_{d}) was calculated from at least three (n≥3) binding curves (each curve giving a single K_{d} value), determined independently and compared to binding curves for insulin. Binding data were analyzed by GraphPad Prism 8 using a non-linear regression and one-site fitting program, which takes the potential ligand depletion into account. The dissociation constant of human ¹²⁵I insulin was set to 0.3 nM. Receptor binding affinities of analogues for IGF-1R were determined by the same methodology as for receptor binding affinity for IR-A and IR-B described above but using mouse embryonic fibroblasts derived from IGF-1R knock-out mice transfected with the human IGF-1R. The cells were a kind gift of Prof. Antonino Belfiore (Catanzaro, Italy). The cells were grown to about 21,000 per well. As a radiotracer human [¹²⁵I]-IGF-1 was used (PerkinElmer Life Science, 2497 Ci/mmol, 44,000 cpm, 0.039 nM). The dissociation constant of human ¹²⁵I-IGF-1 was set up to 0.2 nM.

Cell Toxicity Assay. C2C12 cells were seeded in a flat-bottom, clear, 96-well tissue culture plate at a density of 20,000 cells/well with 200 μL standard growth media. After incubating for 24 h, the media was aspirated and replaced with the treatment (Insulin-DiPBA or Dendrimer-diol) dissolved in standard growth media at 1 mg/mL (n=4/group). A control was performed by treatment with the media. After 24 h incubation, the AlamarBlue assay was performed according to the manufacturer's protocol. UV absorbance was measured at 570 and 600 nm (Temay M200 Pro plate reader) was used to quantify and compare the ratio of viable cells for each treatment. Cell viability was expressed as a percentage of the total AlamarBlue signal relative to the control group.

Isothermal Titration Calorimetry. The binding affinities (K_{eq}) between Insulin-DiPBA and a model GdL-derived diol small molecule under different pH conditions were measured through isothermal titration calorimetry (ITC). All titration experiments were performed at 298 K on a PEAQ-ITC calorimeter (Microcal, Inc.) using a 38 μL syringe and 200 μL cells and consisted of 19 injections. Insulin-DiPBA (3×⁻⁴ or 5×⁻⁴M, loaded in cell) and the model diol (5×⁻³ or 8×⁻³ M loaded in syringe) were dissolved in either 1× phosphate buffered saline (degassed, pH 7.4), 50 mM acetate buffer (degassed, pH 5.0) and 50 mM acetate buffer (degassed, pH 3.5). All raw data were corrected by subtraction of a dilution measurement of the titrate model diol into the corresponding buffer and were then analyzed and graphed using the integrated public-domain software packages of NIPIC, SEDPHAT and GUSSI.

Turbidity Measurements. The turbidity of mixtures at different charge ratios was measured using absorbance measurements at 540 nm on a Temay M200 plate reader. This wavelength was shown to avoid key absorption signatures of the modified insulins and dendrimers and thereby isolate light scattering from aggregated species in solution. Several turbidity analyses were performed, and the complex solutions were prepared according to different evaluated variables. For the study of charge ratio-depended turbidity, stock solutions of insulin and dendrimer derivatives were prepared with 1×PBS. To formulate complexes, appropriate volumes of each compound stock solution and PBS were combined to yield the final desired charge ratio with the final insulin concentration (0.45 mg/mL) kept constant. For the study of glucose-dependent turbidity, glucose-containing buffers were prepared by dissolving glucose in PBS to yield the desired glucose concentration (0 mg/dL, 100 mg/dL, 200 mg/dL, and 400 mg/dL). The stock solutions of insulin and dendrimer derivatives were then prepared in these glucose-containing buffers to a final insulin concentration of 0.05 mg/mL. To formulate a charge-balanced complex, appropriate volumes of each stock solution and related glucose-containing buffers were combined to yield the final desired insulin concentration.

Zeta Potential Measurements. The pH-dependent Zeta-potential analysis was carried out on a Malvern Zetasizer with an auto-titrator attachment. Sample solutions were prepared by dissolving an appropriate amount of each compound with NaCl in water (150 mmol/L) to yield desired sample concentration of 0.5 mg/mL solution. The initial pH was adjusted to 3 by using 1 M HCl (prepared in 150 mM NaCl solution) and titrated with 0.1 M NaOH (prepared in 150 mM NaCl solution) until the pH value reached 11.

Transmission Electron Microscopy. Stock solutions of Insulin-DiPBA and G(6)-Diol were prepared in PBS buffer at a charge-balanced state. Samples were then further diluted in PBS to a final insulin concentration of 0.01 mg/mL. This sample was deposited onto Formvar/Carbon 200 mesh grids (Ted Pella) and wicked. The samples were then briefly washed with DI water to remove salt and allowed to full dry in air, after which negative staining was applied using 2% uranyl acetate. TEM visualization was performed on a JOEL 2011 instrument with an accelerating voltage of 120 kV.

Glucose-Dependent Insulin Release. Insulin-DiPBA and dendrimer-diol were dissolved in PBS and mixed at a charge-balanced state at an insulin concentration of 0.26 mg/mL. Upon mixing, white precipitate formed. The mixture was centrifuged, and the pellet was resuspended in 200 μL of either 1×PBS buffer at pH 7.4 or in 100 mg/dL or 400 mg/dL glucose solutions in PBS at pH 7.4 (n=3/group). At each time point, samples were centrifuged, and the pellet was resuspended in fresh release buffers (200 μL). For cyclic release studies, each day the release buffer was exchanged, alternating between no glucose and 400 mg/dL glucose. Supernatants collected at each time point were diluted and analyzed with Mercodia Iso-Insulin ELISA kit to quantify the amount of Insulin-DiPBA released. These data were fit to a standard curve, accounting for some reduced sensitivity in detecting Insulin-DiPBA relative to native unmodified Insulin.

Circular Dichroism Spectroscopy. Circular dichroism (CD) spectroscopy was used to characterize and compare insulin secondary structure before and after side chain modification. Insulin derivatives (unmodified insulin, insulin DBCO, and insulin-DiPBA were dissolved in 1×PBS at 0.2 mg/mL and loaded into a 1 mm glass cuvette for wavelengths may with J-815 Circular Dichroism Spectrophotometer. CD spectra of unmodified insulin was furthermore used to standardize actual insulin concentration based on Beer's law for the CD absorbance signal at 208 nm.

STZ Mouse Model. A chemically induce diabetes mouse model were established in male C57BL6/J mice (8 weeks, 25 g, Jackson Laboratory) using streptozotocin (STZ), according to common protocols. Mice were fasted for 4 h prior to injection with STZ at 150 mg/kg i.p., dissolved in pH 4.5 citrate buffer. Treated mice were fasted for an additional 0.5 h and were then supplied with food and water as normal. Treated mice were allowed to develop diabetes for 7 d, and diabetes was verified using handheld blood glucose meters (CVS), targeting unfasted blood glucose level (BGL) above 600 mg/dL. All studies were approved by the University of Notre Dame Animal Care and Use Committee. Specifics of these studies are detailed here:

In Vivo Activity Verification. STZ induced diabetic mice were fasted for 8 h on the eighth day following induction, and mice with BGL>350 mg/dL in their overnight fasted state were randomly divided into groups (n=4/group). Mice were injected with 0.1 mg/kg of either unmodified native insulin or Insulin-DiPBA subcutaneously. BGLs for each group were continuously monitored for 6 h after treatment. Mice were fasted during the entire experiment.

Single Day Blood Glucose Control Study. STZ induced diabetic mice were fasted for 8 h on the eighth day following induction, and mice with BGL>350 mg/dL in their overnight fasted state were randomly divided into 3 groups (n=6). Mice were treated with either carrier control (Dendrimer-diol only), the 1:1 Complex, or the 1.5:1 Complex, each at a dose of 10.4 mg/kg Insulin-DiPBA, followed by continuous BGL monitoring for 3 h. An intraperitoneal glucose tolerance test (IPGTT) was performed by injecting glucose solution to each group (1 g/kg in 0.1 mL) to mimic a rapid increase in blood glucose. BGL was then monitored for an additional 3 h following IPGTT. A total of three IPGTT cycles were performed. Mice were fasted during the entire IPGTT period with free access to water. Area under the curve (AUC) for each IPGTT cycle was calculated using the trapezoidal rule and statistically analyzed using GraphPad Prism v9.0.

Multi-Day Blood Glucose Control Study. STZ induced diabetic mice were fasted for 8 h on the eighth day following induction, and mice with BGL>350 mg/dL in their overnight fasted state were randomly divided into 3 groups (n=6). Mice were treated with the 1:1 Complex or 1.5:1 Complex loaded with 10.4 mg/kg Insulin-DiPBA. A control of clinically used Insulin Detemir was administered at a dose of 1 IU/kg and was given at this same dose every subsequent day in the evenings. Continuous BGL monitoring was performed for 3 h after which an intraperitoneal glucose tolerance test (IPGTT) was administered by injecting glucose solution to each group (1 g/kg in 0.1 mL) to mimic a rapid increase in blood glucose. BGL was then monitored for an additional 3 h following IPGTT. Mice were fasted during the entire IPGTT period with free access to water. Mice were fed after IPGTT. At 48 h post treatment, mice were fasted for 5 h followed by an IPGTT challenge for 3 h. Mice were then fed at the end of IPGTT. The IPGTT challenges were repeated on Day 4 and Day 6. Area under the curve (AUC) for each IPGTT cycle was calculated using the trapezoidal rule and statistically analyzed using GraphPad Prism v 9.0.

Multi-Day Serum Insulin Levels. STZ induced diabetic mice were fasted for 8 h on the eighth day following induction, and mice with BGL>350 mg/dL in their overnight fasted state were randomly divided into 6 cohorts (n=5-6). On Day 0, all mice were treated with either saline (3 cohorts) or the 1:1 Complex (3 cohorts) loaded with 10.4 mg/kg Insulin-DiPBA in an overnight fasted state. One cohort from each treatment group was selected on Day 0 and maintained in a fasted state, while other cohorts resumed feeding. For the DO cohort, BGL was measured and 100 μL blood was collected via submandibular bleed for each mouse at 2.5 h after treatment injection (pre-IPGTT baseline). IPGTT was then performed at 3 h after treatment injection (1 g/kg in 0.1 mL) to mimic a rapid increase in BGL. BGL was measured and 100 μL blood was collected via submandibular bleed for each mouse at 0.5 h after GTT (post-GTT). On Day 2 (48 h after treatment injection), both saline and 1:1 Complex groups were fasted for 5 h. BGL was measured and 100 μL blood was collected via submandibular bleed for each mouse (pre-GTT baseline). IPGTT was then performed at 0.5 h after blood collection (1 g/kg in 0.1 mL) to mimic a rapid increase in BGL. BGL was measured and 100 μL blood was collected via submandibular bleed for each mouse at 0.5 h after GTT (post-GTT). This process was repeated again on Day 4. All blood samples were centrifuged to collect serum on the day of collection. Serum insulin concentrations were then quantified using an Iso-Insulin ELISA kit (Mercodia).

Single Day Serum Insulin Kinetics. STZ induced diabetic mice were fasted for 8 h on the eighth day following induction, and mice with BGL>350 mg/dL in their overnight fasted state were randomly divided into 8 cohorts (n=5). All mice were treated with either a dendrimer-diol carrier control (Cohort A, B, C and D) or the 1:1 Complex (Cohort A, B, C and D) loaded with 10.4 mg/kg Insulin-DiPBA. Mice were fed and brought back to their cage after treatment injection on Day 0. On Day 2 (48 h after treatment), all mice were fasted for 5 h. BGL was measured and 100 μL blood was collected via submandibular bleed for all mice from Cohort D (time 0). IPGTT was then performed on all eight groups (1 g/kg, 0.1 mL/mice) to mimic a rapid increase in BGL. At each time point, BGL was measured and 100 μL blood was collected via submandibular bleed for one cohort per treatment, according to the schedule in the table below. This process continued for 3 h. All blood samples were centrifuged to collect serum on the day of collection. Serum insulin concentrations were then quantified using an Iso-Insulin ELISA kit (Mercodia).

TABLE 1
Cohort Blood Glucose Level (BGL) Collection Times

| | Cohort | Cohort | Cohort | Cohort | Cohort |
|---|---|---|---|---|---|
| | E | A | B | C | D |
| Collecting | 0 | 15 | 30 | 45 | 60 |
| Time (min) | 75 | 90 | 120 | 150 | 180 |

Long term blood glucose control study. Healthy mice were first weighed individually and injected STZ following the above protocol. STZ induced diabetic mice were fasted for 8 h on the eighth day following induction, and mice with BGL>350 mg/dL were randomly divided into 4 groups (n=8-10). Mice were treated with either carrier control (saline), daily injections of Insulin Detemir (1 IU/kg), the 1:1 Complex, or the 1.5:1 Complex loaded with 10.4 mg/kg Insulin-DiPBA. Mice were fed after treatment injection and brought back to their cage on Day 0. On BGL measurement days, the daily Insulin Detemir group was injected 7 h prior to fasting, and thus 12 h before BGL measurement. For all other groups, 48 h post treatment injection, mice were fasted for 5 h to adjust for time since last eating, followed with BGL measurement. The same process was repeated at 96 h following treatment injection. After measurement, mice were weighed individually. Mice were then re-dosed with the same formula every 5 days, collecting BGL at 48 h and 96 h following treatment with a 5 h fast; the process was repeated for one month.

Long-Term Biocompatibility Study. Healthy mice were randomly divided into 3 groups (n=5) and treated with either saline, the 1:1 Complex, or the 1.5:1 Complex, each loaded with 10.4 mg/kg Insulin-DiPBA. Each group was then re-dosed with the same treatment every 5 days for 1 month. At the endpoint of the studies, 300 μL of blood was collected via terminal cardiac puncture. All blood samples were centrifuged to collect serum on the day of collection, and serum samples were shipped frozen to IDEXX (North Grafton, MA USA) for a liver and kidney blood chemistry panel. Liver and kidney tissues were fixed in 10% formalin for 2 d, and then transferred to ethanol for paraffin embedding and histological staining using hematoxylin and eosin stain at the Notre Dame Histology core facility.

Diabetic Ossabaw Swine Model. A diabetic model was prepared using 11-14 month old male Ossabaw miniature swine by administration of alloxan, following previously described methods. Alloxan solution was made by adding the powder to a solution of 11 mL 0.9% NaCl and 14 mL NaOH at pH 6.9-7.1. Pigs were fasted ~23 h before alloxan administration at 160 mg/kg. Animals were monitored for 4 weeks to ensure a stable state of fasted hyperglycemia and administered insulin as needed to ensure continued health and well-being. At 3 d prior to treatment, a central venous catheter was inserted to facilitate blood collection. Two days prior to treatment, BGL was collected using a handheld glucometer and 5 mL of blood was collected for subsequent serum insulin quantification. An oral glucose tolerance test (OGTT) was then conducted by administering 2 g/kg dextrose through oral gavage. At 20, 40, 60, 90, 120, and 150 min following OGTT, BGLs were measured, and blood was collected. This process was repeated on the day prior to treatment as well. On the treatment day, all swine (n=6) in an overnight fasted state were administered the 1:1 Complex at a dose of ~0.8-0.9 mg/kg Insulin-DiPBA, depending on body weight, via subcutaneous injection. BGLs were monitored for 20, 40, 60-, 90-, 120-, and 150-min following treatment. At 3 h following treatment, an OGTT was performed as described above with BGL measurements and serum insulin collection. This OGTT process was repeated for days 1-6 following treatment, again measuring BGL and collecting serum for insulin analysis. All collected blood was centrifuged to isolate serum and frozen for subsequent serum insulin concentration analysis using an Iso-Insulin ELISA kit (Mercodia). These studies were conducted by CorVus Biomedical, LLC (Crawfordsville, IN) and approved by a veterinary consultant and the Animal Care and Use committee.

Dosing was determined using the following rationale and derived from the efficacious dose used in mouse studies. To apply a safe and effective insulin derivative dose to swine, the dose used in swine is calculated from the dose used in mice based on published literature to adjust insulin dosing on the basis of body surface area.

$$Estimated {Dose}_{pig} , \frac{mg}{kg} = {Dose}_{m o u s e} , \frac{mg}{kg} \times {( \frac{{Weight}_{mouse} , kg}{{Weight}_{pig} , kg} )}^{( 1 - 0.67 )}$$

For example, for a male swine weighing 59 kg, the dose was estimated based on mouse dose (25 g, 10.4 mg/kg):

$$Estimated {Dose}_{p i g} , \frac{mg}{kg} = 10.41 \frac{mg}{kg} \times {( \frac{0.025 kg}{59 kg} )}^{( 1 - 0.67 )} = 0.802 \frac{mg}{kg}$$

The dose and injection volume for each animal is shown in Table 2.

TABLE 2
Dose and Injection Volume for Swine (Pigs)

| | Pig 1 | Pig 2 | Pig 3 | Pig 4 | Pig 5 | Pig 6 |
|---|---|---|---|---|---|---|
| Weight (kg) | 57.5 | 54 | 59 | 59 | 40.5 | 45.5 |
| Dose (mg/kg) | 0.809 | 0.826 | 0.802 | 0.802 | 0.909 | 0.874 |
| Injection | 17.9 | 17.1 | 18.2 | 18.2 | 14.1 | 15.3 |
| Volume (mL) | | | | | | |

Dynamic Light Scattering. Dynamic Light Scattering (DLS) was carried out to measure the hydrodynamic radius and diffusion coefficients of insulin derivatives on a Malvern Zetasizer. Insulin derivatives were dissolved in PBS (pH 7.4) at 0.52 mM. Solutions of insulin and insulin-DiPBA were filtered with a 0.22 m pore size filter into a disposable cuvette. The size measurement was taken right after filtration to obtain the hydrodynamic radius and diffusion coefficient for each insulin.

Hemolysis Assay. Single donor human red blood cells (10 mL, suspended at 25% dilution of cells to Alsever's solution, Innovative Research) were centrifuged at 500×g for 5 min in a 15 mL conical tube. Supernatant was then aspirated, and a fresh PBS buffer (pH 7.4) was added to the conical tube up to the 10 mL mark line. The tube was inverted gently to resuspend red blood cells. Red blood cell was further diluted by adding 1 ml of the resuspended red blood cell solution to the 24 mL PBS buffer, yielding the final 10% red blood cell in the PBS buffer. Dendrimer-Diol and Insulin-DiPBA stock solutions were prepared at 10, 4 and 2 mg/mL concentration in PBS. For each sample well, 10 μL of each stock solution for both Dendrimer-Diol and Insulin-DiPBA were pipetted into a flat bottom 96-well plates, each sample were loaded in triplicate. For positive control wells, 10 μL of 20% Triton X-100 were added, while for negative control wells, 10 μL of PBS buffer were added. To each sample and control well, 190 μL of the 1% red blood cell solution were added, resulting in final Dendrimer-Diol and Insulin-DiPBA concentration at 500, 200, 100 μg/mL and Triton-X-100 concentration at 1%. These tested concentrations were chosen to be in the range of ~1×-5× of the maximal possible blood concentration of each component, assuming complete and immediate absorption of the entire delivered dose into circulation of ~2 mL of mouse blood. The plate was then incubated at 37° C. for 1 h and then centrifuged at 500×g for 5 min. and 100 μL of supernatant from each well was transferred into a transparent, flat-bottom 96 well plate. Absorbance was measured at 450 nm on a Temay M200 plate reader. The percent hemolysis was then calculated:

$$\% Hemolysis = \frac{{A}_{s a m p l e} - {A}_{P B S}}{{A}_{Triton X} - {A}_{P B S}} \times 100 \%$$

Statistics and Data Analysis. Experimental data was collected above, plotted, and analyzed for each group. For each experiment, data was plotted, and statistical analysis was performed using GraphPad Prism (V9.5.1). For in vitro experiments mean±SD is shown, while for in vivo experiments mean±SEM is shown, customary for in vivo studies. Where noted, statistical comparisons between two groups were performed using a student's t-test, while for studies comparing multiple groups a one-way analysis of variance (ANOVA) was performed with a Tukey multiple comparisons post-hoc test. The figure captions in the main text note sample sizes, error presentation format, and statistical testing, where appropriate.

### B. Example 1: Chemical Synthesis of Insulin Conjugates and Dendrimer Diols

### 1. Abbreviations

NBS is N-bromo-succinimide;
Et₂O diethyl ether;
DBCO is dibenzocyclooctyne;
DCM is dichloromethane;
THF is tetrahydrofuran;
ACN is acetonitrile;
DMF is dimethyl formamide;
CHCl₃ is chloroform;
NaH₂CO₃ is sodium bicarbonate;
MeOH is methanol;
TLC is thin-layer chromatography;
TEA is triethylamine;
eq. or equiv. is equivalents;
min or min. is minute(s);
h or hr. is hour(s);
rt, RT, or r.t. is room temperature; and
sat'd or sat. is saturated.

### 2. Synthesis of Example Insulin-Diboronates

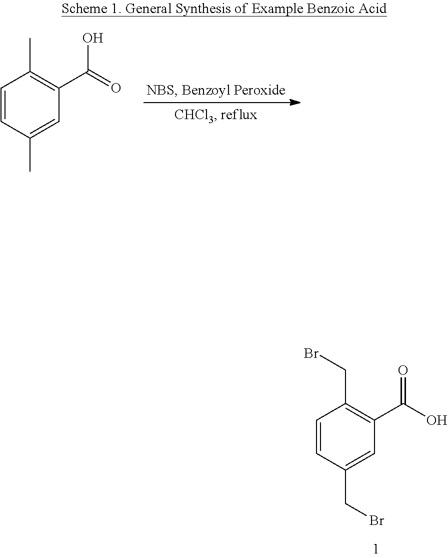

2,5-Bis(bromomethyl)benzoic acid (1): A mixture of 2,5-dimethylbenzoic acid (3 g, 20 mmol), NBS (8.9 g, 50 mmol), and benzoyl peroxide (0.24 g, 1 mmol) was charged into an oven-dried 250 mL round bottom flask and then suspended with 90 mL chloroform. The mixture was then heated to reflux for 4 h and was cooled to ambient temperature. The solvent was removed under reduced pressure and the residue was treated with 90 mL Et₂O. The mixture was filtered, and the filtrate was transferred to a separation funnel, to be washed sequentially with water and brine. The organic layer was then dried with Na₂SO₄, filtered, and concentrated under reduced pressure. The residue was then recrystallized with hexane and ethyl acetate at −20° C., followed by the second recrystallization with DCM and acetone at −20° C. The product was then filtered as a white solid with a yield of 2 g (6 mmol, 30%). ¹H NMR (400 MHz, Chloroform-d) δ 8.10 (d, J=2.0 Hz, 1H), 7.56 (dd, J=7.9, 2.1 Hz, 1H), 7.47 (d, J=7.9 Hz, 1H), 4.96 (d, J=2.2 Hz, 2H), 4.47 (s, 2H).

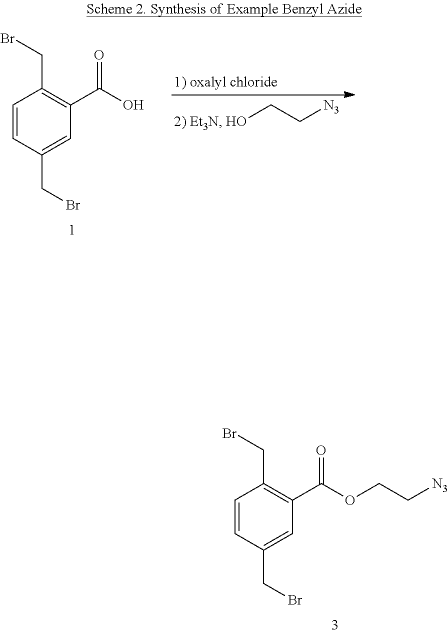 [SMILES: O=C(O)c1cc(CBr)ccc1CBr ; [N-]=[N+]=NCCO ; [N-]=[N+]=NCCOC(=O)c1cc(CBr)ccc1CBr]

2-Azidoethyl 2,5-bis(bromomethyl)benzoate (3): An oven-dried 100 mL round bottom flask was charged with compound 1 (1 g, 3.25 mmol), DMF (20 μL), and dissolved in 15 mL mixed solvent of THF:DCM(1:4). The mixture was then stirred at 0° C. for 10 min before the dropwise addition of oxalyl chloride (1.3 mL, 16.2 mmol). After the addition, the mixture was allowed to stir at ambient temperature for 90 min before solvent was removed under reduced pressure. The residue was then diluted with 20 mL DCM, transferred into an addition funnel, and added dropwise into a stirred solution of compound 2 (0.35 g, 4 mmol) and triethylamine (0.7 mL, 4 mmol) in DCM (20 mL) at 0° C. The mixture was stirred at 0° C. for 2.5 h, and then transferred into a separation funnel, washed sequentially with 1 N HCl (50 mL), water (50 mL), brine (50 mL), and then dried over Na₂SO₄. The solvent was then removed under vacuum and the residue was loaded onto a silica column, eluting with hexane and ethyl acetate for purification. The target product was collected as a transparent oil with yield of 49%. ¹H NMR (400 MHz, Chloroform-d) δ 8.00 (dd, J=4.2, 1.9 Hz, 1H), 7.58-7.41 (m, 2H), 5.01 (d, J=3.2 Hz, 1H), 4.92 (d, J=3.1 Hz, 1H), 4.57 (d, J=3.9 Hz, 1H), 4.48 (dt, J=7.9, 4.3 Hz, 3H), 3.63 (q, J=4.9 Hz, 2H).

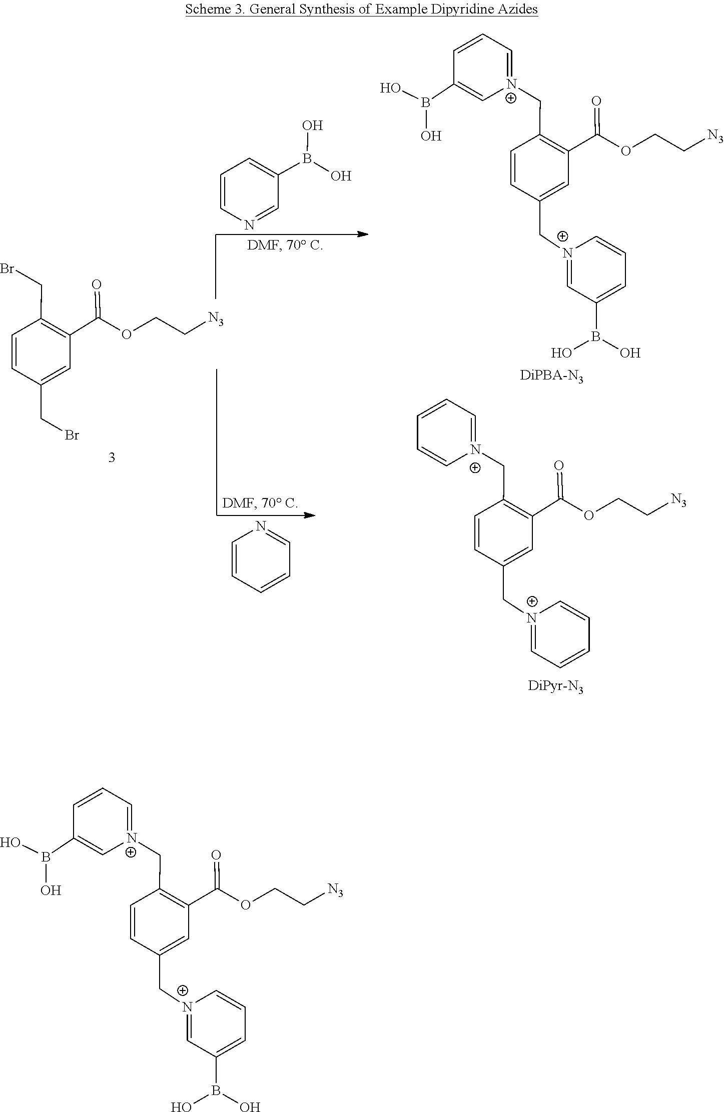

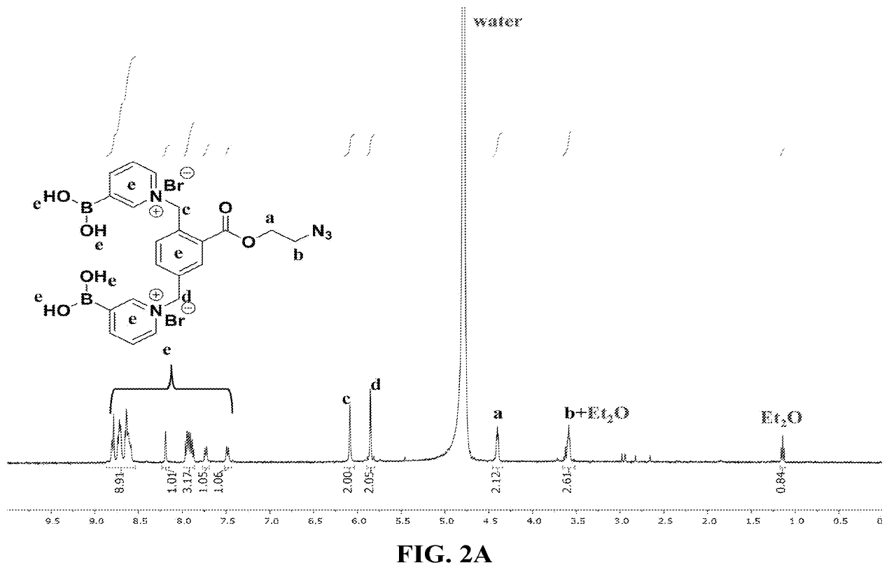

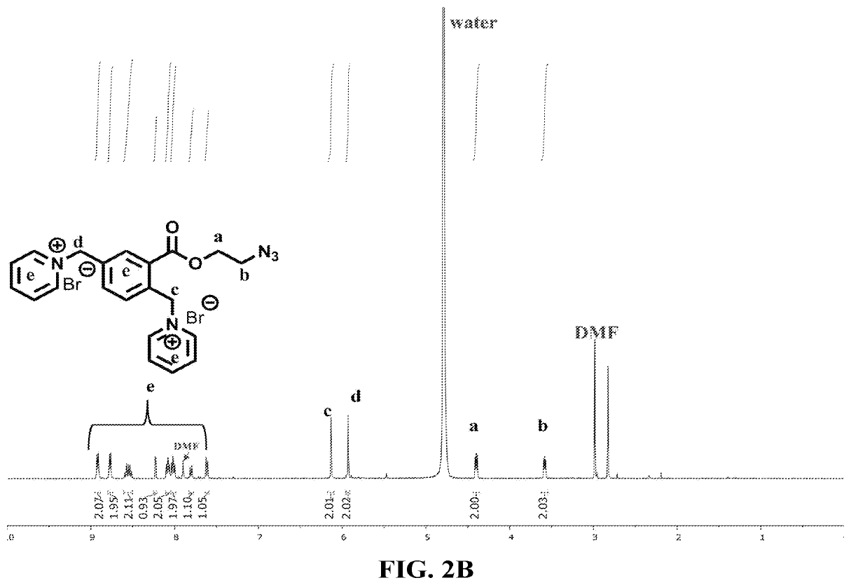

1,1′-((2-)((2-Azidoethoxy)carbonyl)-1,4-phenylene)bis(methylene))bis(3-boronopyridine-1ium) (DiPBA-N₃): Compound 3 (0.6 g, 1.6 mmol) and pyridin-3-ylboronic acid (0.4 g, 3.34 mmol) were diluted with 20 mL dry DMF and the solution was kept stirring at 70° C. overnight. The undissolved solids were recovered by filtration, washed with THF, and diethyl ether, and dried under vacuum to constant weight as the final product with a yield of 90%. ¹H NMR spectrum of DiPBA-N₃ is shown in FIG. 2A. ¹H NMR (400 MHz, Deuterium Oxide) δ 8.86-8.53 (m, 9H), 8.19 (s, 1H), 7.92 (dq, J=13.9, 7.2 Hz, 3H), 7.73 (d, J=7.7 Hz, 1H), 7.48 (d, J=8.1 Hz, 1H), 6.09 (s, 2H), 5.85 (s, 2H), 4.46-4.34 (m, 2H), 3.66-3.52 (m, 2H).

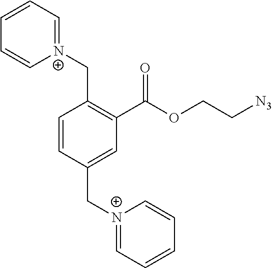 [SMILES: [N-]=[N+]=NCCOC(=O)c1cc(C[n+]2ccccc2)ccc1C[n+]1ccccc1]

1,1′-((2-((2-Azidoethoxy)carbonyl)-1,4-phenylene)bis(methylene))bis(pyridine-1-ium)(DiPyr-N₃): Compound 3 (0.6 g, 1.6 mmol) and pyridine (0.26 g, 3.34 mmol) were diluted with 20 mL dry DMF and the solution was kept stirring at 70° C. overnight. The undissolved solids were recovered by filtration, washed with THF and diethyl ether, and dried under vacuum as the final product with a yield of 90%. ¹H NMR spectrum of DiPyr-N₃ is shown in FIG. 2B. ¹H NMR (400 MHz, Deuterium Oxide) δ 8.95-8.89 (m, 2H), 8.80-8.74 (m, 2H), 8.55 (dt, J=15.9, 7.9 Hz, 2H), 8.23 (d, J=2.0 Hz, 1H), 8.08 (t, J=7.2 Hz, 2H), 8.02 (t, J=7.1 Hz, 2H), 7.80 (dd, J=8.0, 2.0 Hz, 1H), 7.62 (d, J=8.0 Hz, 1H), 6.13 (s, 2H), 5.93 (s, 2H), 4.44-4.36 (m, 2H), 3.62-3.54 (m, 2H).

Insulin-DBCO (I_{DBCO}): A solution of insulin (1 g, 0.17 mmol) in NaHCO₃ (0.1 M, 20 mL) was prepared at ambient temperature, then treated with the solution of DBCO-PEG2-NHS ester (106 mg, 0.19 mmol) in ACN (5 mL). The pH of the above mixture was monitored and maintained at 10.5 for 2 h, and the reaction was then quenched by adjusting the pH to 5.5. The undissolved solids were collected by centrifugation, re-dissolved with 0.1% TFA solution in water, and purified by reversed-phase preparative HPLC (C₈ column with a gradient from Water+0.1% TFA to Acetonitrile). The desired fraction was collected and lyophilized to white powder as the target product (0.6 g, yield: 40%). The desired single-modified species was verified using ESI-MS.

To verify B29-specific modification, the product was first dissolved in a 50 mM ammonium bicarbonate buffer (pH 8.0) at 1 mg/ml. Dithiothreitol (DTT) was dissolved in deionized water to prepare a 500 mM DTT stock solution. The DTT stock solution was added to the insulin solution for final DTT concentration of 5 mM. The reaction mixture was incubated at 50° C. for 50 min to reduce disulfide bonds, following which it was cooled to room temperature and centrifuged to remove solids. Iodoacetamide (IAA) was freshly dissolved in deionized water for a 500 mM IAA stock solution. IAA stock was added to the reduced insulin solution at a final IAA concentration of 15 mM. The reaction mixture was incubated for 30 min at room temperature in the dark to alkylate the reduced cysteines. Unreacted IAA was quenched with addition of DTT stock at a final concentration of 5 mM, incubating for 15 min at room temperature. The mixture was then centrifuged to collect the supernatant. HPLC grade trypsin was first dissolved in 1 mM HCl at 1 mg/mL and added to the protein solution to a molar ratio of 1:100 of trypsin to insulin. The mixture was incubated for 18 h at 37° C. to digest the insulin. The mixture was then cooled to room temperature and trypsin was quenched by acidification with TFA (0.4% vol/vol, pH<2). The result was then analyzed with Liquid Chromatography/Mass Spectrometry. The LC-MS instrument consisted of a Dionex Ultimate 3000 Rapid Separation UPLC system equipped with a Dionex Ultimate 3000 autosampler and a Dionex Ultimate 3000 photodiode array detector coupled with a Bruker MicrOTOF-Q II quadrupole time-of-flight hybrid mass spectrometer using Hystar 3.2 software. The Bruker electrospray ionization source was operated in the positive ion mode with the following parameters: end plate offset voltage=−500 V, capillary voltage=2000 V, and nitrogen as both a nebulizer (4 bar) and dry gas (8 L/min flow rate at 180° C. temperature). Mass spectra were accumulated over the mass range 50-3000 Da. The sample was analyzed on a Dionex Acclaim™ RSLC 120 C8 column (2.2 m, 120 Å, 2.1 mm i.d.×100 mm) with a 15 min-LC gradient (2-min hold at 95% A/5% B, followed by a 11-min linear gradient to 5% A/95% B, an 0.1-min linear gradient to 95% A/5% B, and then 1.9-min hold at 95% A/5% B where A=0.1% formic acid in water; B=0.1% formic acid in acetonitrile).

Stock solutions of DBCO-modified insulin (i.e., “insulin-DBCO” or “I_{DBCO}”) (215 mg, 0.034 mmol) in DI water (4.5 mL) and a substituted dipyridine (43.5 mg, 0.068 mmol) or an unsubstituted dipyridine (2.5 mg, 0.0048 mmol) in DI water (0.5 mL) were prepared at ambient temperature. These two solutions were then mixed, and the pH of the mixture was monitored and maintained at 7 for 2 h before being purified by reversed-phase preparative HPLC (Cis column with a gradient from Water+0.1% TFA to Acetonitrile).

Insulin-DiPBA (I_{DiPBA}): Stock solutions of I_{DBCO} (215 mg, 0.034 mmol) in DI water (4.5 mL) and DiPBA-N₃ (43.5 mg, 0.068 mmol) in DI water (0.5 mL) were prepared at ambient temperature. These two solutions were then mixed, and the pH of the mixture was monitored and maintained at 7 for 2 h before being purified by reversed-phase preparative HPLC (C8 column with a gradient from Water+0.1% TFA to Acetonitrile). The desired fraction was collected and lyophilized to white powder as the target product (178 mg, yield: 83%).

Insulin-DiPyr (I_{DiPyr}): Stock solutions of I_{DBCO} (25 mg, 0.004 mmol) in DI water (4.5 mL) and compound DiPyr-N₃ (2.5 mg, 0.0048 mmol) in DI water (0.5 mL) were prepared at ambient temperature. These two solutions were then mixed, and the pH of the mixture was monitored and maintained at 7 for 2 h before being purified by reversed-phase preparative HPLC (Cis column with a gradient from Water+0.1% TFA to Acetonitrile). The desired fraction was collected and lyophilized to white powder as the target product (18 mg, yield: 70%).

### 3. Synthesis of Example Dendrimer-Diols

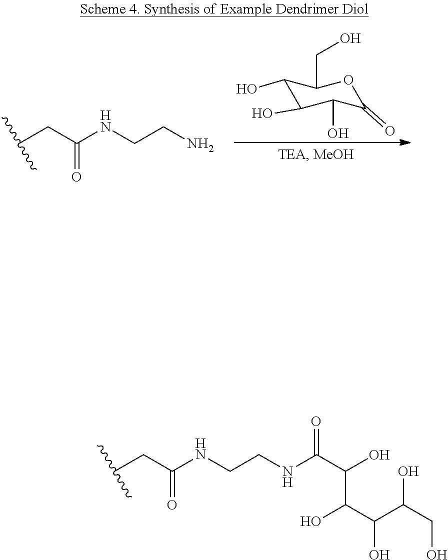 [SMILES: *CC(=O)NCCN ; O=C1OC(CO)C(O)C(O)C1O ; *CC(=O)NCCNC(=O)C(O)C(O)C(O)C(O)CO]

Polyamidoamine (PAMAM) dendrimer-NH₂ (250 mg; Generation G2, 16 arms, G4, 64 arms, or 6 G6, 256 arms) were purchased from Dendritech® and mixed with glucono-δ-lactone (GdL) (1.2 eq to NH₂ groups) were dissolved in methanol (250 mL). Triethylamine (TEA, 0.3 ml) was then added to the reaction mixture and was stirred at room temperature for 7 d. The reaction mixture was then concentrated under reduced pressure to remove methanol. The crude product was dissolved in deionized water, loaded into regenerated cellulose dialysis tubing (MWCO=3,500 Da) and dialyzed for 1 d. The final product was lyophilized, yielding a white powder.

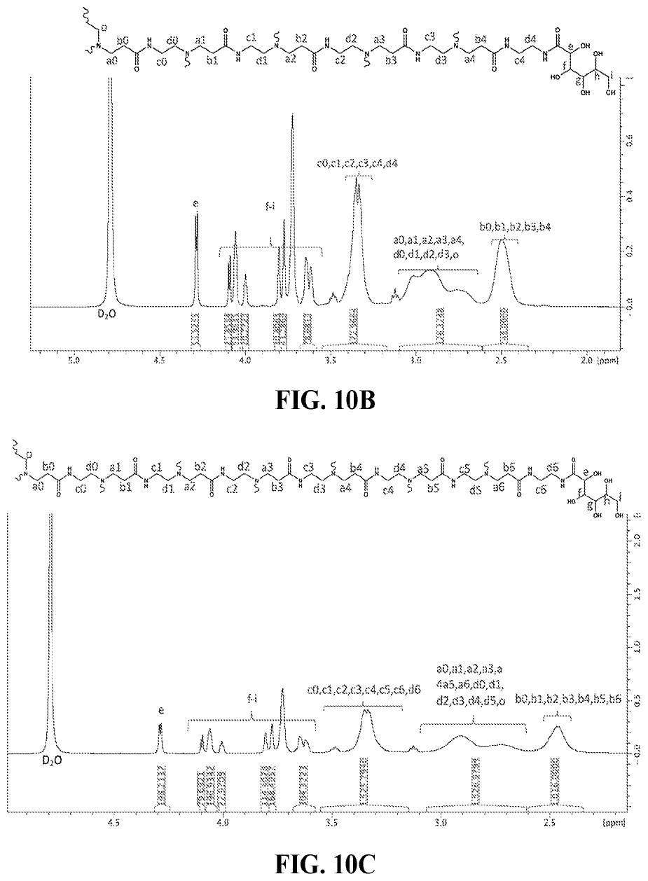

Dendrimer-Diol (G2): ¹H NMR spectrum shown in FIG. 10A. ¹H NMR (400 MHz, D₂O) δ 4.28 ppm (d, J=3.6 Hz, 16H), 4.09-3.58 (bs, 80H), 3.5-3.2 (bs, 88H), 3.2-2.7 (bs, 84H), 2.7-2.4 (bs, 56H).

Dendrimer-Diol (G4): ¹H NMR spectrum shown in FIG. 10B. ¹H NMR (400 MHz, D₂O) δ 4.28 ppm (d, J=3.6 Hz, 64H), 4.1-3.6 (bs, 320H), 3.5-3.2 (bs, 372H), 3.2-2.7 (bs, 376H), 2.7-2.4 (bs, 248H).

Dendrimer-Diol (G6): ¹H NMR spectrum shown in FIG. 10C. ¹H NMR (400 MHz, D₂O) δ 4.28 ppm (d, J=3.6 Hz, 256H), 4.1-3.6 (bs, 1280H), 3.5-3.2 (bs, 1524H), 3.2-2.7 (bs, 1528H), 2.7-2.4 (bs, 1016H).

### C. Example 2: Characterization and Functional Assessment of Insulin Conjugates

### 1. Material Design and Synthesis

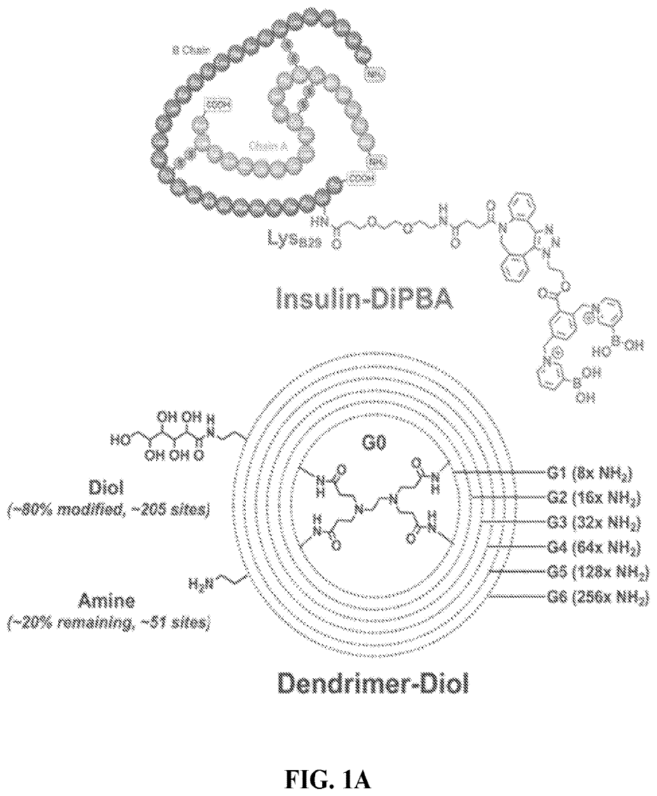

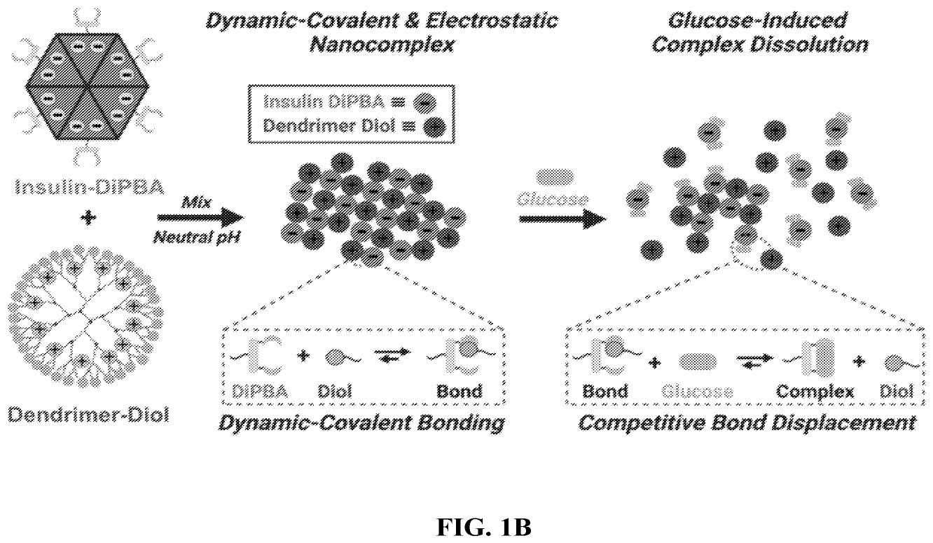

The design inspiration for this approach took concepts from three different clinically evaluated insulin therapeutics to achieve a long-lasting and glucose-responsive depot. A leading option for basal insulin therapy, Insulin Glargine, forms a depot by subcutaneous nanoprecipitation following injection in a pH 5 suspension because of its roughly neutral isoelectric point; slow enzymatically driven depot re-solubilization offers protracted basal availability and ~24-36 h duration of action. NPH Insulin, an intermediate-acting insulin with clinical use dating back to the 1940's, forms a depot with ~24 h duration of action with protraction from the electrostatic complexation of insulin and a positively charged biopolymer protamine. Meanwhile, the first glucose responsive insulin used pre-clinically (MK-2640) modified insulin with oligosaccharides to leverage sugar-binding proteins as depots for competition mediated displacement and insulin release. As such, the envisioned design here was to couple features of subcutaneous nanoprecipitation, electrostatic complexation with a macromolecular carrier, and molecular scale interactions susceptible to competition from free glucose to yield a long-lasting and glucose-responsive insulin depot (FIGS. 1A-1B).

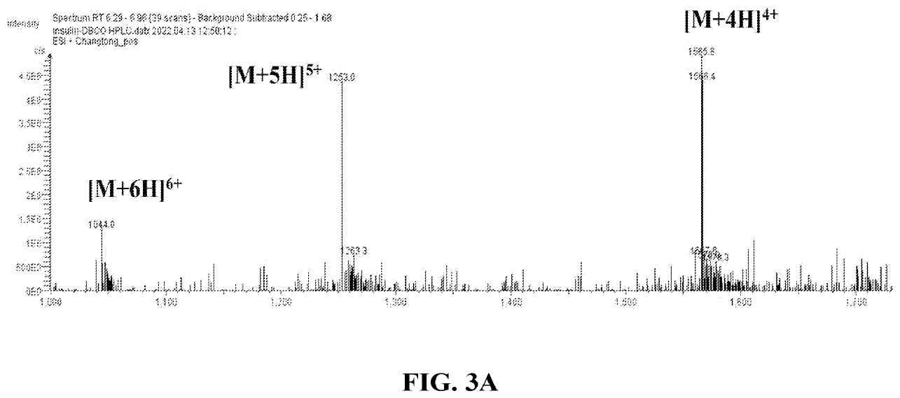

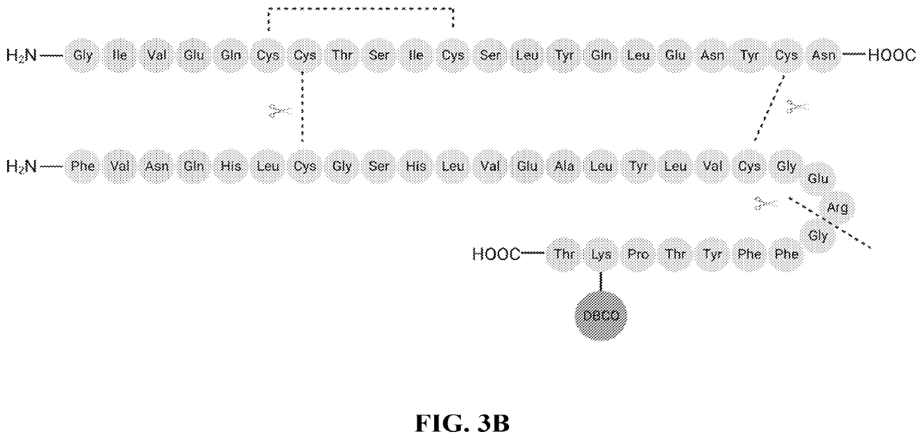

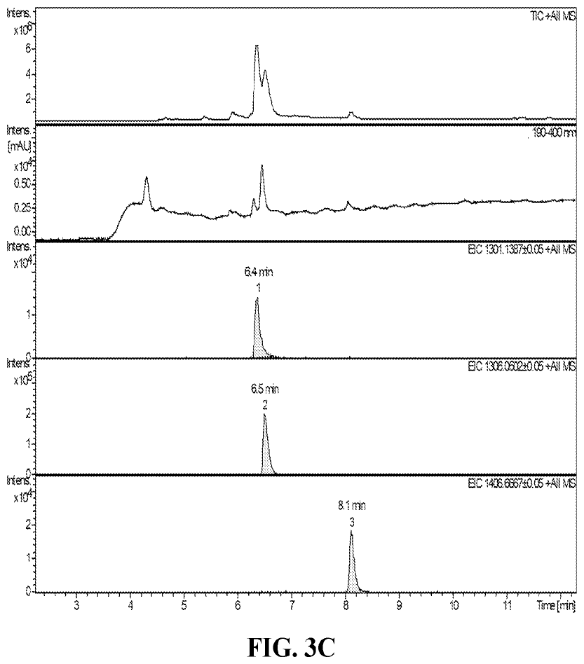

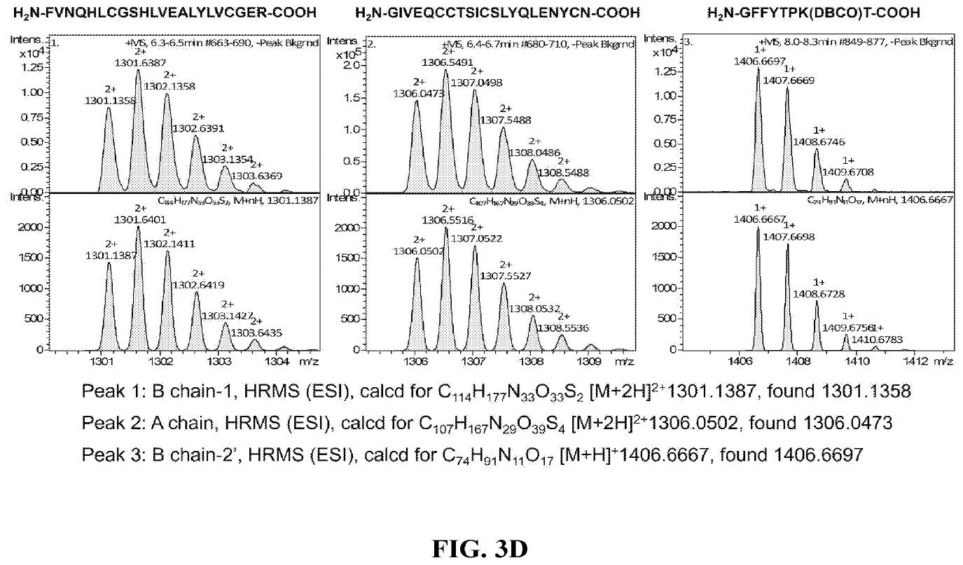

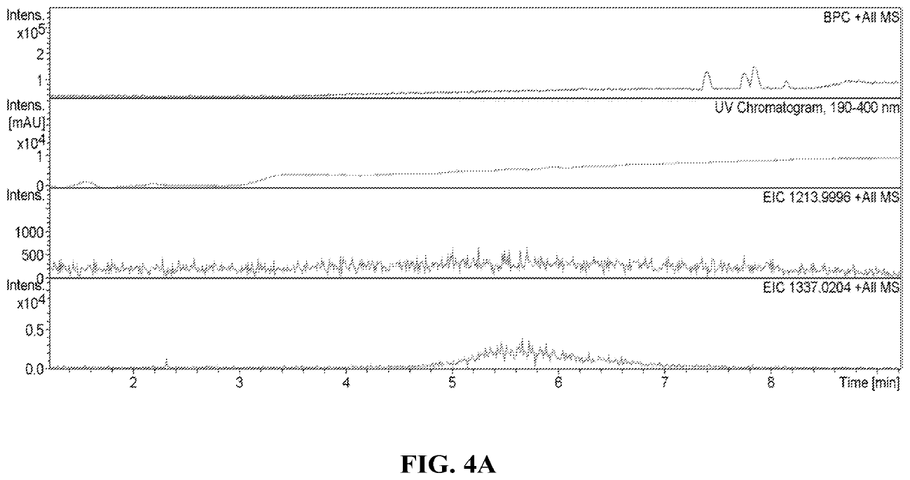

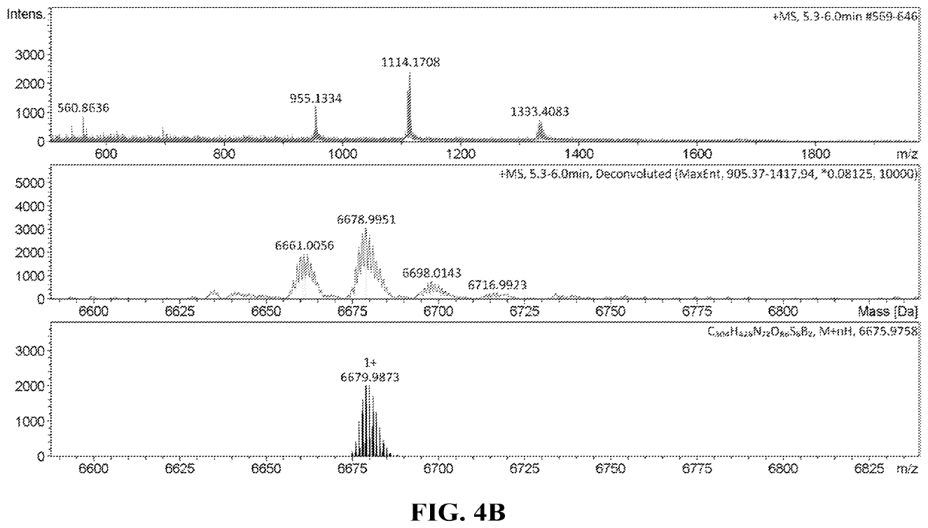

Insulin was first modified with the reported DiPBA motif to endow prosthetic glucose-responsive functionality. Insulin has three primary amines for modification; reaction at the ε-amine of the B29 lysine residue may be enhanced relative to the primarily amines of the A1 and B1 N-terminal positions by controlling the pH of the amide bond-forming reaction. The B29 lysine is also where insulin is modified with a C₁₄ myristic acid in Insulin Detemir, a clinically used long-lasting basal variant. The direct modification of the ε-amine of the B29 lysine using a related DiPBA motif bearing a carboxylic acid was not feasible at pH 11 due to DiPBA degradation, likely by protodeboronation, under basic reaction conditions. As such, a two-step approach was implemented wherein insulin was first modified with Dibenzocyclooctyne-PEG2-N-hydroxysuccinimidyl ester (DBCO-PEG2-NHS ester) under pH 11 conditions, and then subsequently a DiPBA-azide compound (FIG. 2) could be attached via strainpromoted alkyne-azide cycloaddition, or so-called copper-free “click” chemistry (FIG. 1A). Reversed phase preparative HPLC was performed after both DBCO and DiPBA modification steps to isolate the single-modified insulin product. The effectiveness of site-specific modification by this two-step approach was confirmed using digestion of insulin with DTT and trypsin followed by high-resolution LC-MS/MS analysis of the three resulting peptide fragments, which confirmed exclusive DBCO modification at the B29 site (FIGS. 3A-3D). The ensuing “click” reaction proceeds readily to produce the final DiPBA-modified product (FIGS. 4A-4B). Overall, this procedure results in a 33% yield of Insulin-DiPBA from recombinant human insulin.

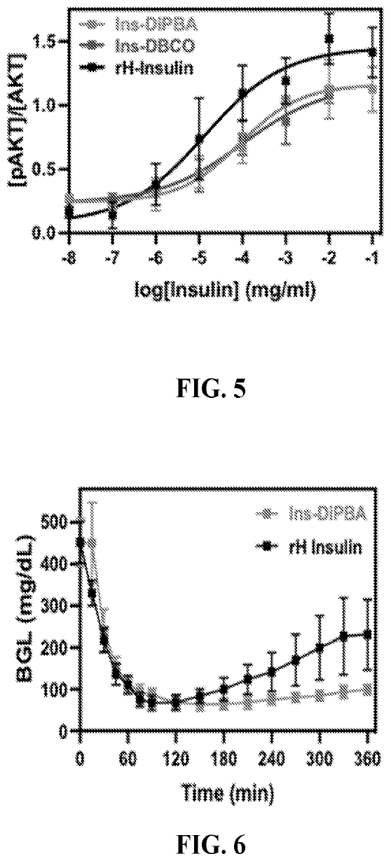

The activity of Insulin-DiPBA was next assessed through an in vitro cell activity assay (FIG. 5). This assay, performed in model C2C12 myoblast cells, quantifies insulin receptor activation reflected in phosphorylated AKT (pSer473) vs. total AKT. The EC₅₀ measured by this assay was 13 μg/L for recombinant insulin; addition of a DBCO linker led to an increase in EC₅₀ of an order of magnitude (101 μg/L), with subsequent DiPBA addition having similar impact on in vitro potency (EC₅₀=89 μg/L). Thus, modification with DBCO led to reduced insulin potency in vitro that was maintained upon subsequent DiPBA attachment via click chemistry. As cell assays were performed in glucose-containing media, the DiPBA would appear to still signal in its glucose bound state, though the modification itself does impact activity. In another measure of insulin activity/potency, Insulin-DiPBA alone was assessed in STZ diabetic mice (FIG. 6). At an identical dose of 0.1 mg/kg (equal to 3 IU/kg of native insulin), Insulin-DiPBA depressed blood glucose at a rate comparable to unmodified insulin in the first 90 min after administration, suggesting potency to be initially matched. Yet, whereas blood glucose increased in the insulin group after ~120 min, Insulin-DiPBA had a longer duration of action with blood glucose rising much more slowly after an initial nadir at ~150 min. Such protraction is common when receptor binding affinity is reduced, owing to the reduced rate of insulin clearance from circulation; the physicochemical modification of insulin may also interfere with the binding of insulin-degrading enzyme to its substrate. As such, modification may be responsible for extending the duration of action relative to unmodified insulin in vivo, though does not appear to have a dramatic impact on potency reflected in the initial rate of blood glucose correction.

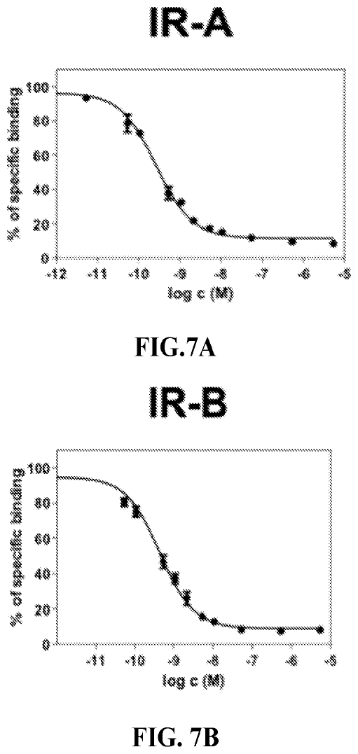

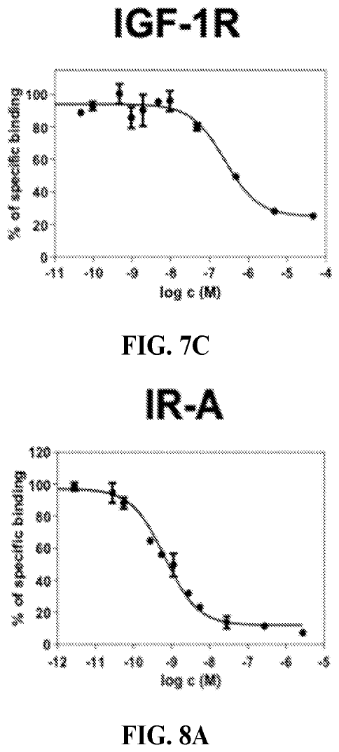

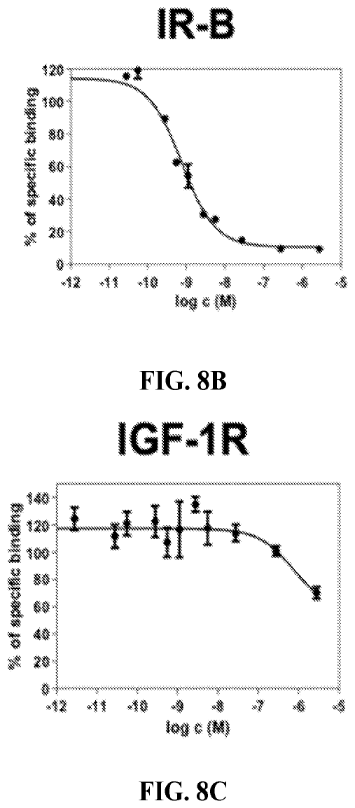

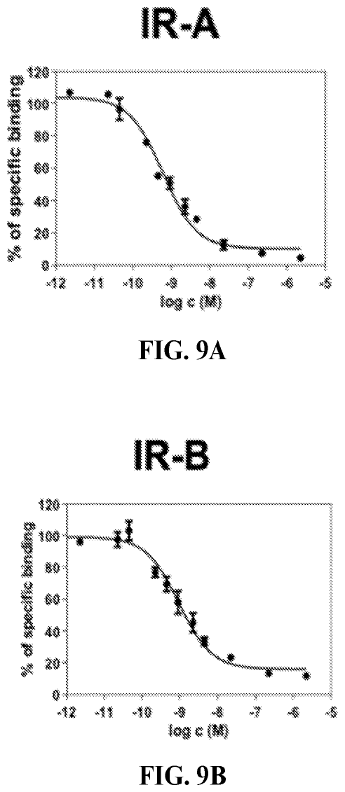

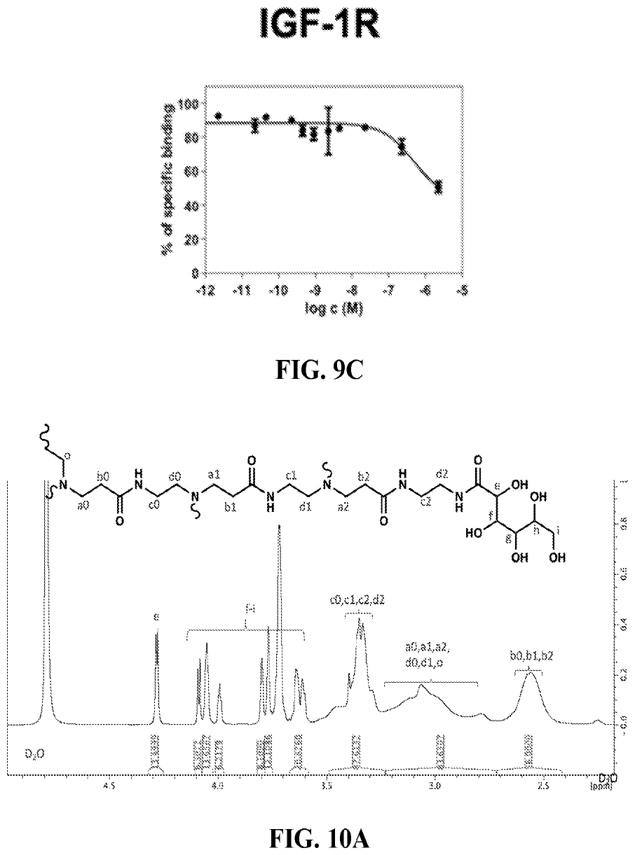

To further understand the impact of DiPBA modification on insulin receptor affinity, radiolabeled displacement studies were performed to assess the affinity of these B29-modified insulins compared to native insulin in binding to insulin receptor A (IR-A) and insulin receptor B (IRB) (FIGS. 7A-7C, 8A-8C, 9A-9C). Binding affinities of insulin and B29-modified variants Insulin-DBCO and Insulin-DiPBA to insulin receptor A (IR-A), insulin receptor B (IR-B), insulin-like growth factor 1 (IGF-1R) are shown in Table 3.

TABLE 3
Binding affinities of insulin and B29-modified variants Insulin-
DBCO and Insulin-DiPBA to insulin receptor A (IR-A), insulin receptor
B (IR-B), insulin-like growth factor 1 (IGF-1R).¹

| | IR-A | | IR-B | | IGF-IR | |
|---|---|---|---|---|---|---|
| | K_{d} (nM) | % | K_{d} (nM) | % | K_{d} (nM) | % |
| Insulin (n = 3) | 0.19 ± 0.06 | 100 | 0.36 ± 0.01 | 100 | 200 ± 45 | 0.05 |
| Ins-DBCO (n = 3) | 0.50 ± 0.13 | 38 | ±0.18 | 50 | 630 ± 252 | 0.016 |
| Ins-DiPBA (n = 3) | 0.59 ± 0.09 | 32 | 0.86 ± 0.24 | 42 | 825 ± 404 | 0.012 |
| IGF-1 (n = 3) | — | — | — | — | 0.10 ± 0.04 | 100 |

¹Tabulated affinities are expressed relative to insulin or IGF-1. Relative binding affinity is defined as (K_{d} of the native hormone/K_{d} of analogue) × 100 (%).

The affinities of Insulin-DBCO and Insulin-DiPBA in binding to IR-A were 38% and 32% (FIGS. 7A, 8A, and 9A) that of native insulin, respectively; for IR-B, these were also reduced to 50% and 42% of native insulin (FIGS. 7B, 8B, and 9B). Insulin Detemir also has reduced potency due to insulin receptor binding affinity that is ~25% that of unmodified insulin, pointing to the expected impact of B29 modification on insulin potency. As such, the reduced affinity arising from B29 modification aligns with expectations and is likely to underlie the reduced cell signaling potency and in vivo protraction of function observed. Modifying insulin may also unintentionally increase mitogenicity of the protein via aberrant activation of insulin-like growth factor receptor (IGF-1R). For example, the modification of the C-terminal B chain in Insulin Glargine is known to enhance mitogenicity through IGF-1R binding. However, here the B29 modification resulted in lower binding affinity for IGF-1R than even unmodified human insulin (FIGS. 7C, 8C, and 9C); mitogenicity via IGF-1R binding is therefore unlikely. Yet, as mitogenicity of insulin is mechanistically complex, it is possible that synthetic modification of insulin may still enhance its mitogenicity through some other mechanism and this topic would need to be further explored in development of any therapeutic insulin.

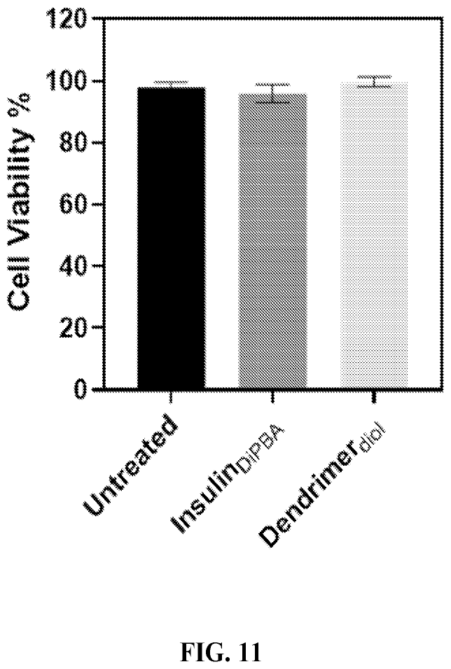

As insulin has a net-negative charge, PAMAM dendrimers were chosen as carriers to leverage their cationic character in facilitating electrostatic complexation. The predictable and globular structure of PAMAM dendrimers, with very low polydispersity, was specifically targeted for this work due to protein-mimetic size and structural features to ensure reproducible function of this envisioned nanocomplex platform. These dendrimers also have well-defined and addressable end-groups for facile modification. PAMAM dendrimers of Generation 2, 4, and 6 were next modified by reaction with glucono-δ-lactone (GdL) on their peripheral amino groups, following methods used for the preparation of complexes using PBA-diol bonding. In each case, ~80% of terminal amines were modified with the GdL-derived diol, as confirmed by ¹H NMR (FIGS. 10A-10C). Both Insulin-DiPBA and the G6 Dendrimer-diol exhibited excellent cytocompatibility in vitro (FIG. 11).

### 1. Nanocomplex Formation

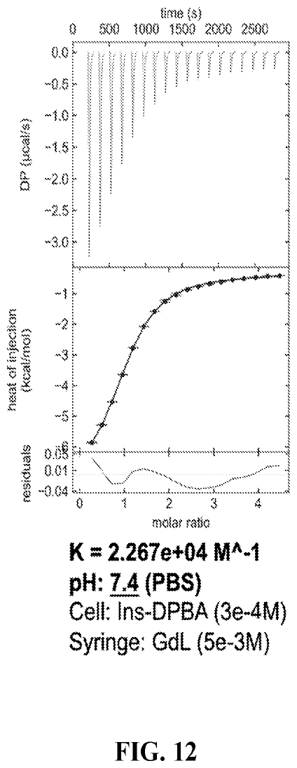

Two complementary interactions were envisioned to prepare Insulin-Dendrimer nanocomplexes. The inherent differences in net charge were first targeted to drive nanoprecipitation and depot formation when the net-negative Insulin-DiPBA and positive Dendrimer-Diol were mixed, like the mechanism underlying function of NPH Insulin. Added to that was the dynamic-covalent DiPBA-diol interaction to stabilize the Insulin-Dendrimer nanocomplexes and render their interactions glucose-responsive. To first confirm that Insulin-DiPBA retained its ability to recognize the GdL-derived diol, isothermal titration calorimetry (ITC) was performed at pH 7.4 to assess binding between Insulin-DiPBA and a small molecule GdL-derived diol (FIG. 12). The binding affinity (Keq) was measured to be 2.3×10⁴ M⁻¹ at pH 7.4. Interestingly, this is ~4 times higher affinity than was measured for the small molecule DiPBA-diol interaction. Attachment to insulin could alter both the presentation and associative dynamics for the dynamic-covalent interaction, giving rise to higher binding affinities.

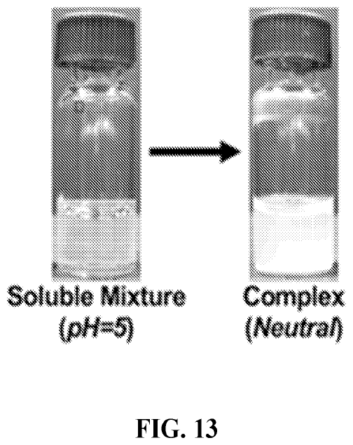

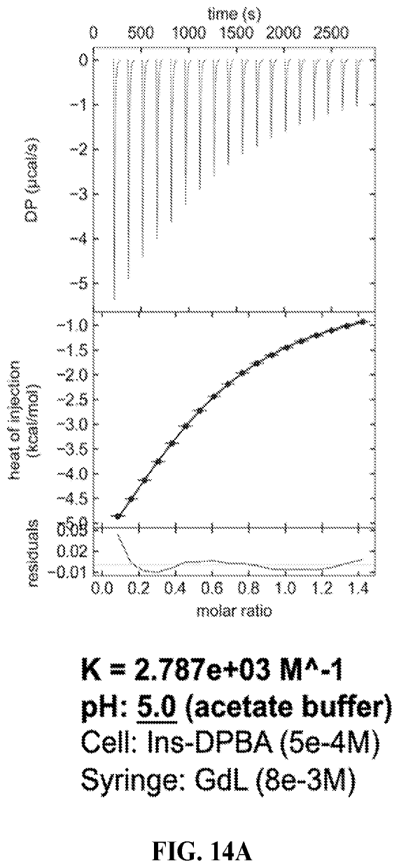

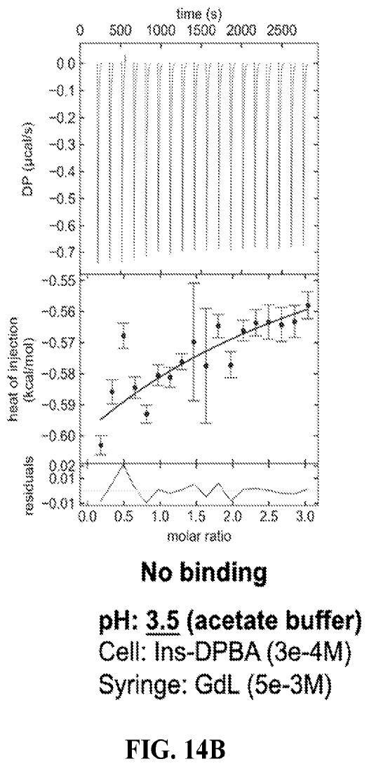

Insulin-DiPBA and Dendrimer-Diol mixtures were soluble at pH 5, yet formed visible precipitates at pH 7.4, noted by increased sample turbidity (FIG. 13). This solubility profile is similar to that of Insulin Glargine, which is injected at pH 5 and forms a nanoprecipitate depot in the body due to its neutral isoelectric point. The pH-induced shift in solubility likely results from enhanced electrostatic screening at neutral conditions as well as higher affinity DiPBA-diol bonding. The latter point is supported by the pH dependence of the bonding between Insulin-DiPBA and GdL-derived diol, where affinity measured by ITC was reduced by an order of magnitude (2.8×10³ M⁻¹) at pH 5, and no binding was recorded at pH 3 (FIGS. 14A-14B). The pKa for the two boronic acids on the DiPBA was previously reported to be 4.5 (pKa,1) and 7.4 (pKa,2). PBA-diol interactions are well-known to form readily at pH levels at or above the pKa of the boronate, wherein it may adopt its tetrahedral and charged conformation. As such, these pH-dependent trends in affinity are expected.

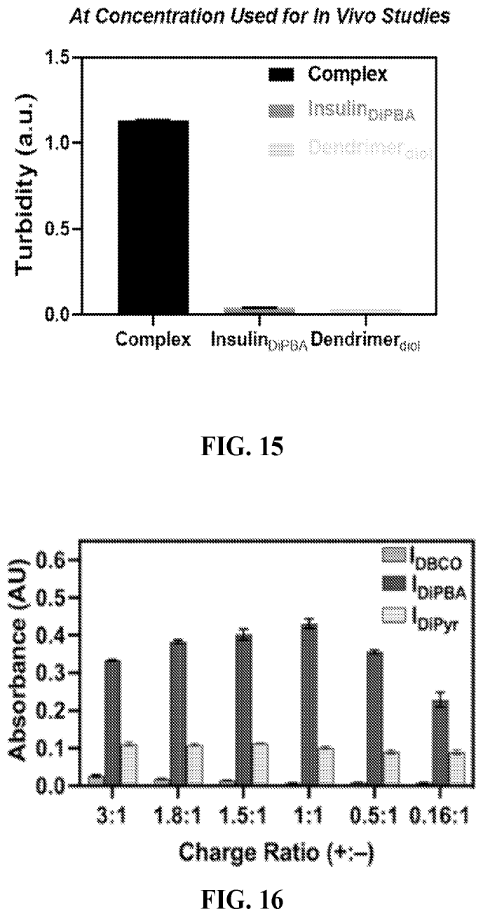

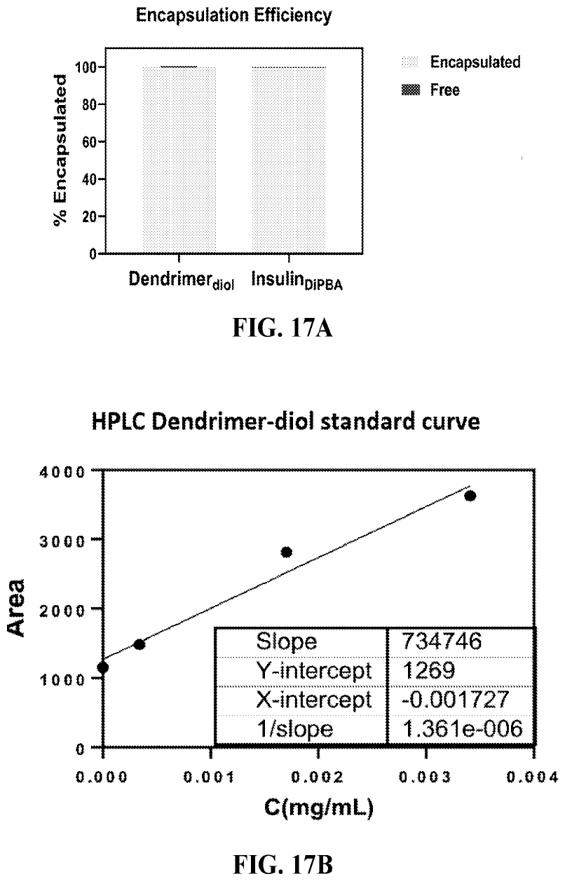

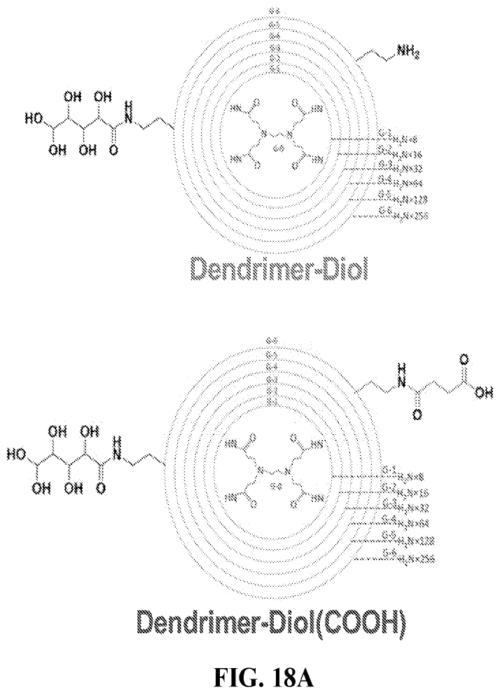

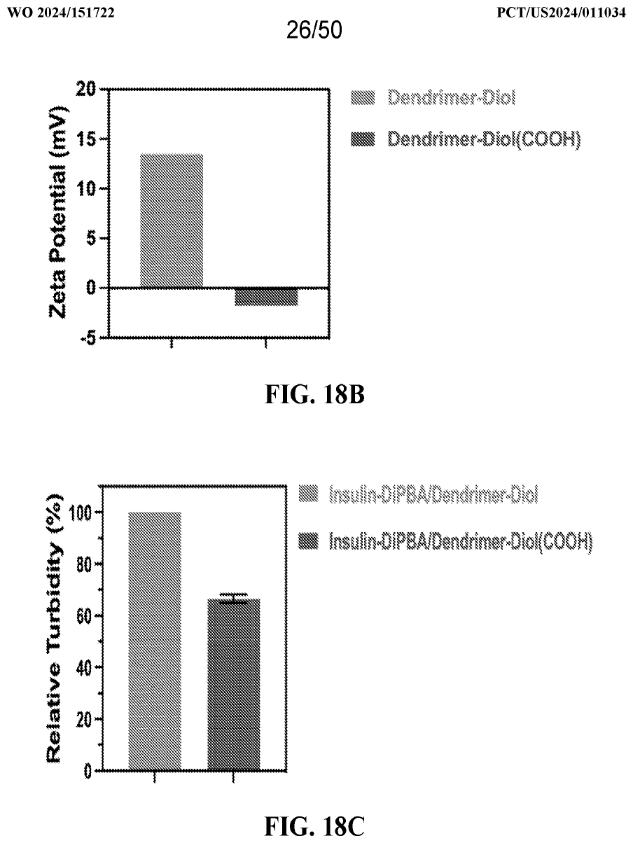

Alone at pH 7.4, Insulin-DiPBA and Dendrimer-Diol were fully soluble with no measurable turbidity (FIG. 15). The mixing of Dendrimer-Diol (+) and Insulin-DiPBA (−) under these same conditions revealed maximal complex formation at a charge ratio of 1:1, as evidenced by a measurement of sample turbidity over a range of mixing ratios (FIG. 16). Both Insulin-DiPBA and Dendrimer-Diol were essentially fully incorporated in the formed nanocomplexes at 1:1 charge balance, as verified by analysis of the soluble fraction following separation of the formed complexes via centrifugation (FIGS. 17A-17B). For clarity, the molar ratio of Dendrimer-Diol to Insulin-DiPBA is 1:17 at 1:1 charge balance; this equates to a ratio of 1:2.8 in terms of moles of dendrimer to insulin hexamers.

The net charge of the various insulin derivatives at physiological pH was determined based on the pKₐ of ionizable groups. Glutamic acids (E) and the C-terminal carboxylic acids of the A- and B-chains each contribute a negative charge (−1), while arginine (R), lysine (K), and the N-terminal amino groups of the A- and B-chain contribute a positive charge (+1). The contribution of lysine (K) was adjusted to account for the only lysine (B29 residue) being the site of prosthetic modification, thus converting its charged ε-amine to an uncharged amide; the prosthetic group itself introduced charge at this site as follows: DiPBA (roughly neutral), diPyr (+2), and DBCO (0). Accordingly, the three modified insulins were estimated to have a net charge as follows: Insulin-DiPBA (−3), Insulin-DiPyr (−1), and Insulin-DBCO (−3).

The net charge of Diol-modified PAMAM dendrimers was determined for the surface-presented amino groups for dendrimers. Table 4 shows the estimates of the net charge/mole of dendrimers. For the G2 dendrimer, it does not possess the same density of charges as higher generations, and as such the contribution from internal tertiary amines is less clear. Thus, its net charge was calculated for both extrema where only surface charges were present (+3) and an alternate scenario where surface amines as well as internal tertiary amines are charged (+30).

TABLE 4
Estimates of Net Charge/Mole of Dendrimers.

| Generation of | | Number of | |
|---|---|---|---|
| Dendrimer | Number of | Surface | Net |
| (surface | Diol/mol (80% | —NH₂/mol (80% | charge/ |
| groups) | modification) | modification) | mol |
| G6 (256) | 205 | 51 (outside layer) | +51 |
| G4 (64) | 51 | 13 (outside layer) | +13 |
| G2 (16) | 13 | 3 (outside layer) | +3 |
| G2 (8)* | 5 | 25 (overall) | +30 |

*Net charge/mole was calculated for circumstances where only surface charges were present (+3) and an alternative scenario where surface amines as well as internal tertiary amines are charged (+30).

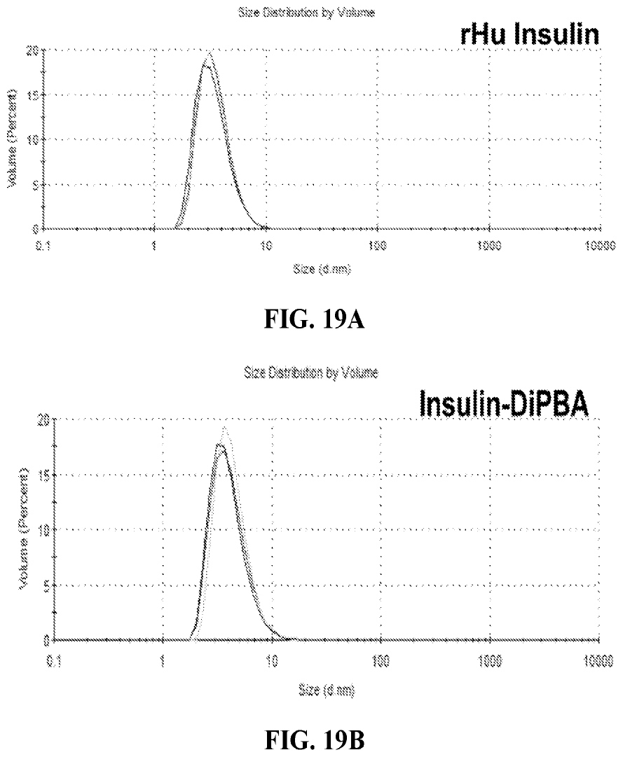

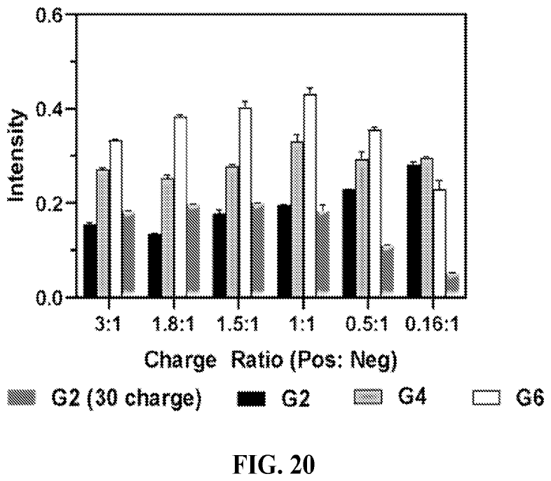

Control insulin variants consisting of insulin modified with a dibenzocyclooctyne (DBCO) prosthetic group (“Insulin-DBCO”) and insulin modified with a dipyridinium prosthetic group (“Insulin-DiPyr”) did not form the same level of complex formation at any charge ratio, supporting a role for DiPBA-diol crosslinking alongside electrostatics in stabilizing the nanocomplex. When peripheral amines on the Dendrimer-Diol were converted to carboxylic acids, effectively mayceling the charge on the dendrimer, complexation still occurred when mixing with Insulin-DiPBA at the same molar ratio as the 1:1 complex though not to the same extent based on turbidity (FIGS. 17A-17C). The data for nanocomplexation, together with ITC studies, point to DiPBA-diol interactions being the primary means of nanocomplexation, with electrostatics serving in a stabilizing role. G6 PAMAM dendrimers (~58 kDa) have a diameter of ~6.7 nm, roughly comparable to the dimensions of an insulin hexamer (~36 kDa) with a diameter of ~5.6 nm. The assumption of a hexameric state for Insulin-DiPBA was supported by dynamic light scattering (FIGS. 19A-19B), comparing diffusion coefficients to that for native insulin without zinc removed as well as results in published work. As such, from the outset G6 PAMAM dendrimers were hypothesized to be a better match to formulate with insulin for depot formation. This was further confirmed when comparing the resulting nanoprecipitation to that from other amine terminated PAMAM generations (G2 and G4), which had reduced turbidity even at a state of charge balance (FIG. 20).

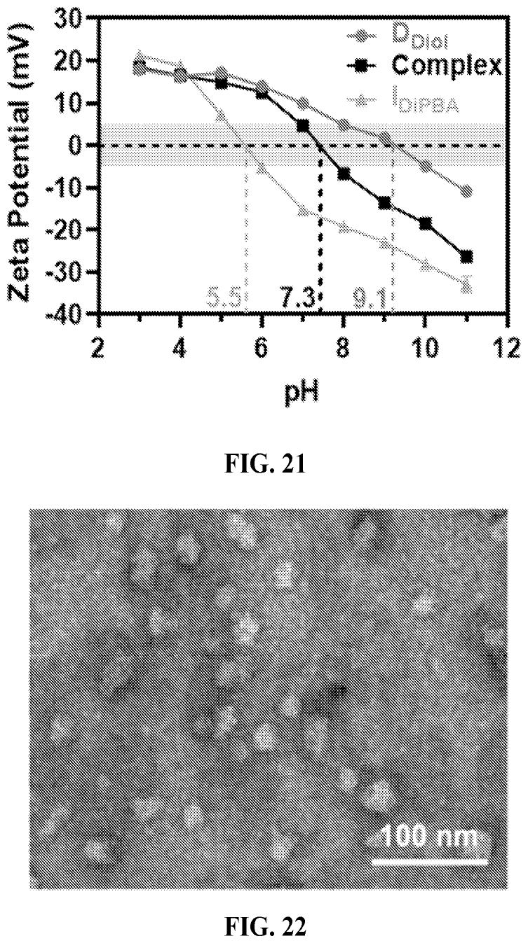

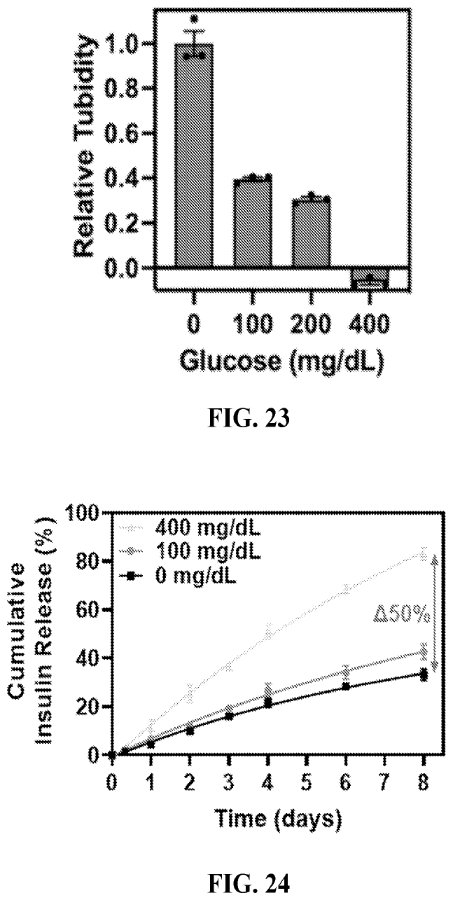

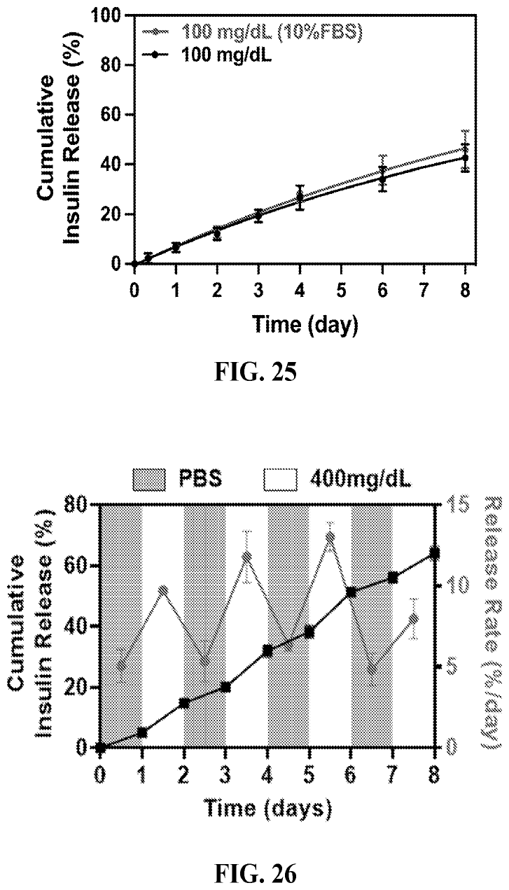

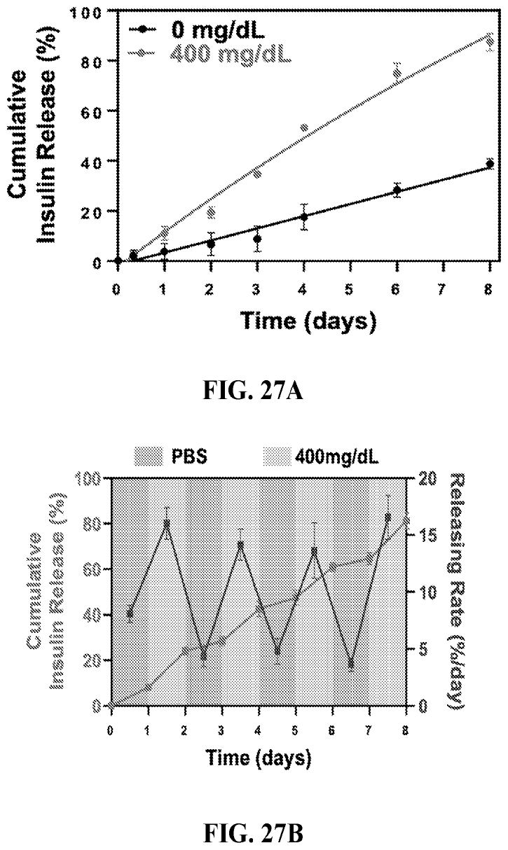

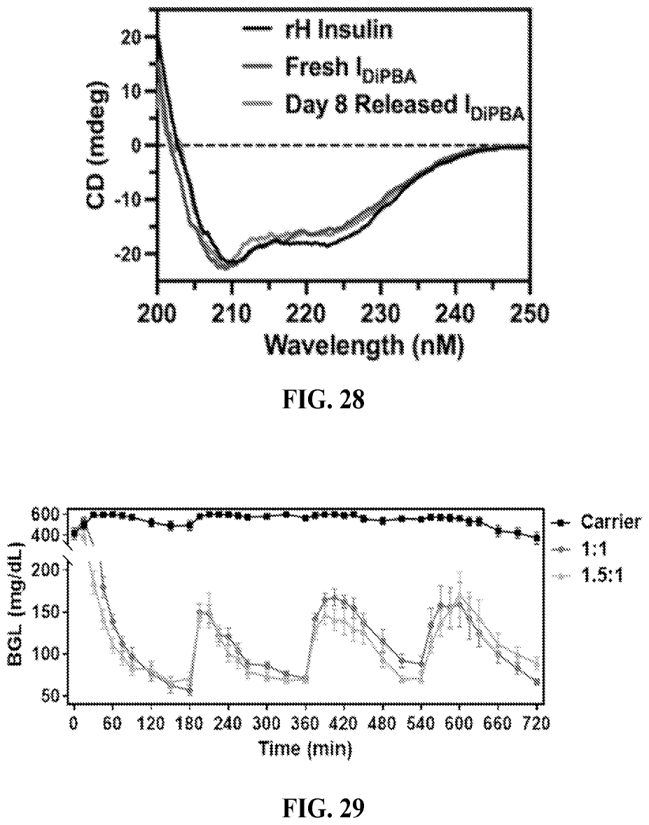

Zeta potential collected in the course of a pH titration revealed isoelectric points of ~5.5 for Insulin-DiPBA and ~9.1 for the Dendrimer-Diol, with the 1:1 charge-balanced complex being net-neutral at pH ~7.3 (FIG. 21). These data further support the methods used to estimate the charge state of the two components to achieve balance, confirming electrostatic stabilization under neutral pH conditions. Nanocomplex diameters of ~30-40 nm were observed by transmission electron microscopy in the dry state (FIG. 22); these diameters are on the same order as those formed by Insulin Glargine when introduced into neutral conditions. When forming nanocomplexes in the presence of glucose, the extent of aggregation was reduced, with no detectable complex formed when glucose levels were raised to up to level of 400 mg/dL, resembling hyperglycemic conditions (FIG. 23). The impact on nanocomplex formation due to competition from glucose further supports a primary role for DiPBA-diol dynamic-covalent interactions in the initial nanocomplex formation of the Insulin-Dendrimer formulation. When complexes were pre-formed and exposed to bulk conditions of 400 mg/dL, the release of free insulin was accelerated relative to its release in a bulk buffer without glucose or in buffer containing a normal (100 mg/dL) level of glucose (FIG. 24). Sustained release was observed in both cases, with total cumulative release after 8 days of ~83% in the 400 mg/dL case versus ~33% in buffer and ~43% in 100 mg/dL glucose. It is expected that some insulin release occurs even in the absence of glucose under these dilution conditions, resulting from a shift in the binding equilibrium of DiPBA-diol interactions as well as slow nanocomplex erosion. Notably, the addition of protein to the release media (10% fetal bovine serum) to resemble conditions in vivo had no impact on release (FIG. 25). The release rate was also roughly doubled upon repeated cycling between no- and high-glucose levels (FIG. 26). Of note, when the complexes were compared at close to charge balance (+:− of 1.5:1), insulin release in both glucose-free and glucosecontaining media was enhanced, with similar doubling of the release rates when cycled between no glucose and 400 mg/dL (FIGS. 27A-27B). Circular dichroism (CD) spectroscopy of Insulin-DiPBA after 8 days of release from the Insulin-Dendrimer nanocomplex revealed no change in characteristic α-helical secondary structure compared to freshly dissolved recombinant insulin or fresh Insulin-DiPBA (FIG. 28).

### D. Example 3: Physiological Evaluation of Insulin-Dendrimer Nanocomplexes in Mice

### 1. Determination of Functional Formulation and Dose in Mice

Mouse dosing was determined iteratively by adjusting the formulation parameters and amount of insulin administered to maximize the duration of action and the glucose-responsive function of the formulation. These studies informed aspects of formulation design and the tolerated insulin doses in arriving at the eventual dose used in the mouse studies.

The Insulin-Dendrimer nanocomplex, loaded with low insulin dose, was first evaluated in STZ-induced diabetic mouse model for single-day glucose-responsive insulin delivery. In this study, unmodified insulin (0.17 mg/kg) was administered as a control and compared to a 1:2 Complex loaded with 0.17 mg/kg Insulin-DiPBA as well as a 3.3:1 Complex loaded with either 0.17 mg/kg or 3.5 mg/kg Insulin-DiPBA. Mice were challenged with three intraperitoneal glucose tolerance tests (IPGTT). At a low insulin dose (0.17 mg/kg), groups treated with unmodified insulin failed during the first IPGTT cycle with BGLs returning to their pre-treatment baseline. Mice treated with all three complexes were able to correct BGL to their pre-treatment baseline throughout all IPGTT cycles. Importantly, no hypoglycemia was observed for any of the doses explored. BGL of mice treated with complexes were then continuously monitored for additional days until their corrective function stopped. By comparison, the 1:2 Complex and 3.3:1 Complex formulated with 0.17 mg/kg of Insulin-DiPBA had a ~50% reduction in fasting BGL one day after treatment injection. It was not until day 3 that mice treated both complexes returned to their original hyperglycemic state. Thus, even at an identical dose to the unmodified insulin, the Insulin-Dendrimer complexes were able to provide extended blood glucose correction without hypoglycemia. The 3.3:1 complex dosed at 3.5 mg/kg, meanwhile, showed no hypoglycemia in spite of a 20× increase in the insulin dose and provided robust blood glucose correction for at least 3 d following administration.

The Insulin-Dendrimer formulations were next optimized for delivery of higher insulin doses and various charge ratios (+:−). The 3.3:1 Complex formulated with either 3.5 or 7 mg/kg Insulin-DiPBA was evaluated in STZ mice. The 3.3:1 Complex formulated with 3.5 mg/kg Insulin-DiPBA failed by day 3 while that formulated with 7 mg/kg Insulin-DiPBA failed by day 5. Another charge unbalanced ratio of a 1:9 Complex loaded with 14 mg/kg Insuli-DiPBA, was also evaluated. Even though loaded with an extremely high dose of Insulin-DiPBA, the 1:9 complex demonstrated severe yet non-lethal hypoglycemia, but only offered blood glucose correction only up to Day 3. This result suggested that a more charge-unbalanced formulation leads to a less stable complex that could not fully trap insulin in the depot. However, a formulation closer to charge balance demonstrated longer functional time even at a lower total insulin dose.

A 1:2 Complex, nearing charge balance, was formulated with either 7, 10 and 14 mg/kg Insulin-DiPBA. In this case, mice treated with the complex formulated with both 7 and 10 mg/kg Insulin-DiPBA started to fail on Day 5, while mice treated with complex formulated with 14 mg/kg Insulin-DiPBA started to fail on Day 6. At this near charge-balanced ratio, mice treated with all three doses were slightly hypoglycemic on Day 0 following injection. Accordingly, while the 1:2 complex was relatively more stable for sustained insulin release from the depot over multiple days, it was still not stable enough, as indicated by initial hypoglycemia of treated mice.

Finally, the charge-balanced 1:1 Complex was formulated with either 3.5 or 10.4 mg/kg Insulin-DiPBA and evaluated in STZ mice. At the charge balanced ratio, complex formulated with both 3.5 and 10 mg/kg Insulin-DiPBA did not lead to hypoglycemia throughout the study. Mice BGL were maintained within normal glycemic range for both doses; mice treated with the 1:1 complex formulated with 10 mg/kg Insulin-DiPBA showed especially great control on BGL until Day 6. These results suggested that a charge-balanced ratio between Insulin-DiPBA and Dendrimer-Diol is crucial to formulate a stable complex with sustained and well-controlled insulin release for blood glucose correction over multiple days.

To achieve longer therapeutic duration while preventing hypoglycemia in mice, the Complex was further optimized at or near its charge-balanced ratio. The 1:1 Complex, 1.5:1 Complex, and 1:2 Complex were formulated with 10 mg/kg Insulin-DiPBA and evaluated in vivo. Here, 10 mg/kg was chosen since this dose showed longer duration of therapeutic function while minimizing potential of hypoglycemia. With the same insulin dose, the 1:2 Complex seemed to have weaker control over BGL, indicated by slight hypoglycemia of mice on Day 0 while in blood glucose correction by Day 5. Accordingly, excess insulin relative to dendrimer in the formulation does not lead to prolonged control. The 1:1 Complex and 1.5:1 Complex showed comparable therapeutic functions throughout the study: both complexes did not induce hypoglycemia in mice throughout the study and achieved blood glucose correction until Day 6. It is worth noting that even though the average BGL of mice treated with the 1.5:1 Complex were in the normoglycemic range, a couple mice were slightly hypoglycemic on Day 0, which further supported the statement that charge balanced ratio is indeed a crucial factor in this platform. Based on otherwise comparable performances, the 1:1 Complex and 1.5:1 Complex formulated with 10 mg/kg Insulin-DiPBA were then brought to other long-term studies for better comparison.

The Insulin-Dendrimer platform was designed to address both electrostatic (charge ratio) and dynamic-covalent (DiPBA-diol) interactions. After demonstrating that a charge-balanced ratio is a key feature of a stable complex, the importance of dynamic covalent bonding was also evaluated. In this case, the Dendrimer-Diol was formulated with Insulin-DiPBA, unmodified insulin, or Insulin-DBCO, all at a charge-balanced ratio. The three charge-balanced complexes were formulated with 3.5 mg/kg insulin and evaluated in vivo. Here, complexes were formulated with 3.5 mg/kg insulin derivatives to avoid undesired overdosing in the case that electrostatic force alone was not sufficient to trap insulin derivatives in the depot. Upon mixing, Insulin-DiPBA and Dendrimer-Diol formed a colloidal suspension with visible aggregation and turbidity. Insulin and Dendrimer-Diol, with electrostatic forces alone, remained clear but formed a slightly more viscous solution. Insulin-DBCO and Dendrimer-Diol, with electrostatic forces only though with higher charge density compared the formulation with native insulin, also formed a colloidal suspension. Complexes prepared from native insulin and Insulin-DBCO resulted in severe hypoglycemia in mice on Day 0 and failed to offer blood glucose correction by Day 1. By comparison, the Complex prepared with Insulin-DiPBA did not lead to hypoglycemia and offered longer therapeutic duration. This suggested that electrostatic force alone is not sufficient to trap insulin in the depot, and as such both electrostatic forces and dynamic-covalent bonding were required necessary for long-term therapeutic functions without initial hypoglycemia.

### 2. Blood Glucose Correction in Mice

Stable Insulin-Dendrimer nanocomplexes at charge ratios (+:−) of 1:1 and 1.5:1 were next explored for single day blood glucose correction in STZ-induced diabetic mice subjected to multiple glucose challenges. These two formulations were evaluated in tandem for early studies as they demonstrated the highest complexation by turbidity measurements along with glucoseresponsive insulin release. The STZ mouse model recreates clinical features of hyperglycemia and insulin deficiency of type 1 diabetes. The Insulin-Dendrimer formulations for these and subsequent rodent studies were performed at an optimal insulin dose of 10.4 mg/kg.

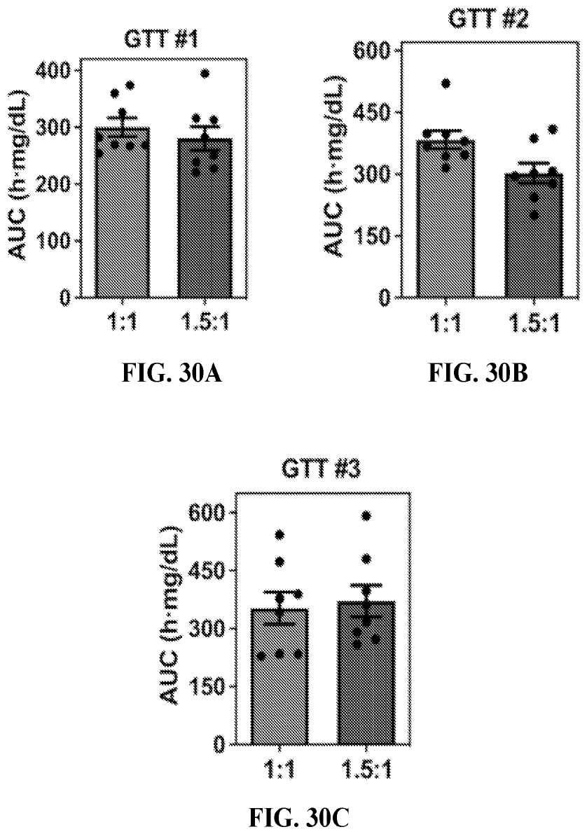

Complexes or a carrier control consisting of only the Dendrimer-Diol were injected subcutaneously (t=0) in overnight-fasted diabetic mice; both complexes restored blood glucose to within a normal physiological range (60-180 mg/dL for mice) over the ensuing 3 h after dosing (FIG. 29). Upon initial dosing, average blood glucose levels reached 57 mg/dL in the 1:1 treatment group; though mice remained stable and alert, these values indicate moderate hypoglycemia that may arise from some extent of insulin burst release upon injection and compression of the liquid formulation under the skin. By comparison, the same extent of hypoglycemia was not observed for the 1.5:1 formulation in this study, where the dendrimer carrier component was at a greater relative ratio. The Dendrimer-Diol carrier control showed no impact on blood glucose, with these mice remaining hyperglycemic for the duration of the study. A control of Insulin-DiPBA at the same dose used in the Insulin-Dendrimer nanocomplex was not feasible, as alone this dose of Insulin-DiPBA is over its lethal dose; this likewise offers indirect support for retention of Insulin-DiPBA in the subcutaneous depot when combined with Dendrimer-Diol. Intraperitoneal glucose tolerance tests (IPGTT) were next performed every 3 h for three cycles while monitoring blood glucose. Both complexes corrected blood glucose following each of three administered IPGTT rounds, with normoglycemia still maintained at 12 h following treatment and three IPGTT cycles. Blood glucose levels at this point for the 1:1 (67 mg/dL) and 1.5:1 (89 mg/dL) formulations were well within the normal range for a fasted healthy mouse. Area under the curve (AUC) after each challenge was quantified (FIGS. 30A-30B), showing comparable response for both formulations. AUC values were also comparable across all three IPGTT cycles.

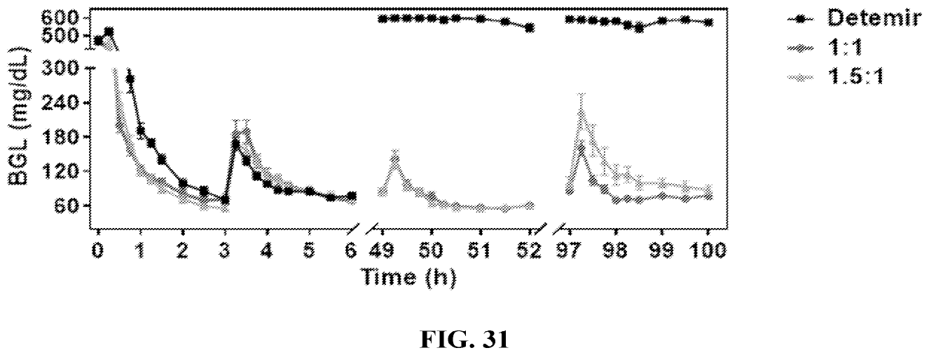

Subcutaneous injection was chosen for evaluation of this technology, as the most effective and most used site for insulin due to consistent uptake in a self-administered setting. However, for a glucose responsive delivery approach, it is noted that interstitial glucose levels are typically lower than plasma glucose levels and have ~10 minutes of lag time in humans, and alternate sites (e.g., intramuscular) may thus be appropriate to consider. It is furthermore possible that interstitial glucose may disrupt the initial nanocomplex formation following injection. A comparison of data presented here for the full formulation at an Insulin-DiPBA dose of 10.4 mg/kg (FIG. 31) with data for Insulin-DiPBA alone at a dose of 0.1 mg/kg reveals similar post-injection nadir values yet a much longer duration of action for the formulation, suggesting that any initial burst of insulin following injection is likely only a small fraction of the injected dose. Moreover, though the DiPBA structure was designed to be more glucose specific than typical PBA chemistries, which bind better to the diol presented on the dendrimer than it does to glucose (FIG. 12) it likely also binds to diols or analytes in the tissue and this could also contribute to the displacement and release of Insulin-DiPBA in vivo.

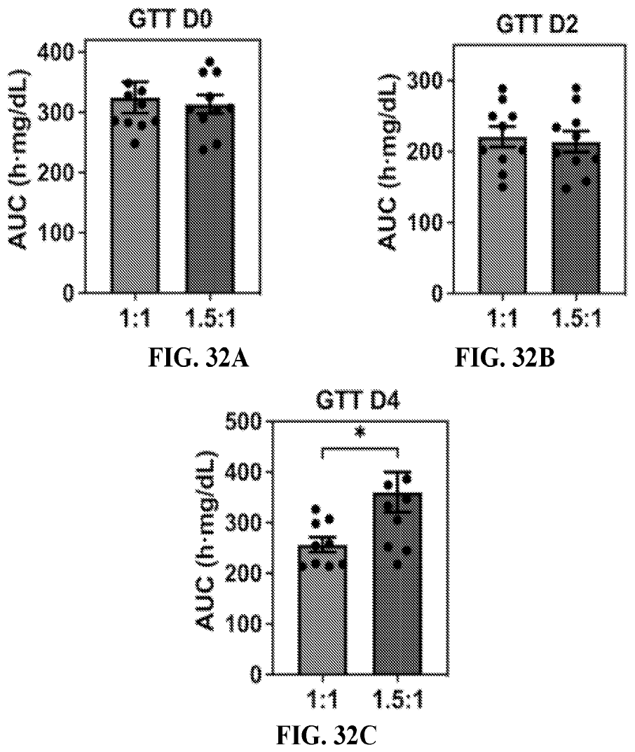

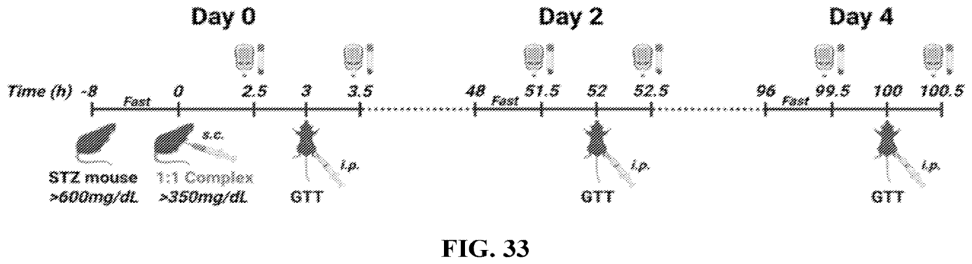

Examples of insulin delivery approaches that correct blood glucose in response to multiple challenges in a single day have been previously reported. Here, with sustained function at 12 h following treatment, a subsequent study was thus performed to test long-term glucose-responsive function of the Insulin-Dendrimer nanocomplex formulations subjected to glucose challenge on day 0, 2, and 4 (FIG. 31); these studies were performed against a control of daily administration of Insulin Detemir, a clinically used long-lasting basal insulin that achieves protraction by binding to circulating serum albumin. The daily dosing of the Insulin Detemir control was chosen to be potency-matched to reach the same blood glucose level at 3 h after administration in overnight-fasted mice, similar to methods used to determine insulin potency in rabbits that form the basis of modern day “International Units” (IU) convention. Insulin Detemir offered similar correction following the day 0 challenge in overnight-fasted mice, reaching blood glucose levels of 71 mg/dL after 3 h, which was comparable to treatment with the 1:1 (72 mg/dL) and 1.5:1 (60 mg/dL) complexes. However, the daily administration of Insulin Detemir did not sustain blood glucose control when this was not combined with overnight fasting. AUC values were quantified for the two formulations (FIGS. 32A-32B), with the 1:1 charge complex demonstrating greater responsiveness to IPGTT at day 4 following treatment. AUC values were also comparable across all days, and similar to the values obtained for the prior study with multiple IPGTT cycles administered in a single day. Though both ratios performed comparably, the improvement in response seen in AUC values for the 1:1 formulation at day 4 may be due to a lower excess of diol sites from less Dendrimer-Diol in the formulation, enhancing the ability of glucose to compete at later stages of the study when there is less Insulin-DiPBA remaining for competition-mediated release. By day 6, however, blood glucose had returned to a hyperglycemic state for mice that were treated with both the 1:1 and 1.5:1 Insulin-Dendrimer nanocomplexes (FIG. 31). However, treatment with both complexes demonstrated sustained blood glucose control for ~5 d with repeated response to IPGTT.

### 1. Glucose-Triggered Depot Insulin Release

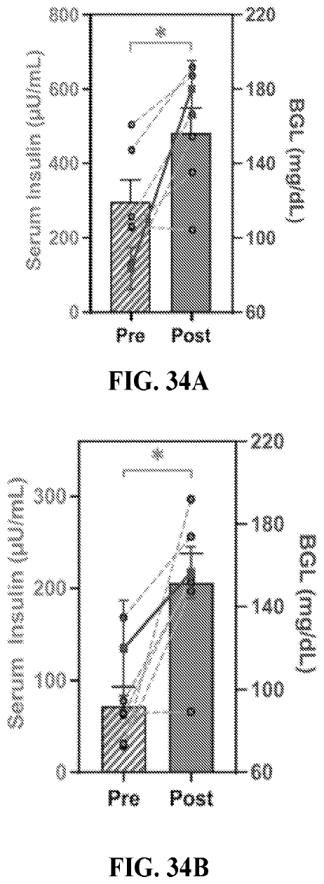

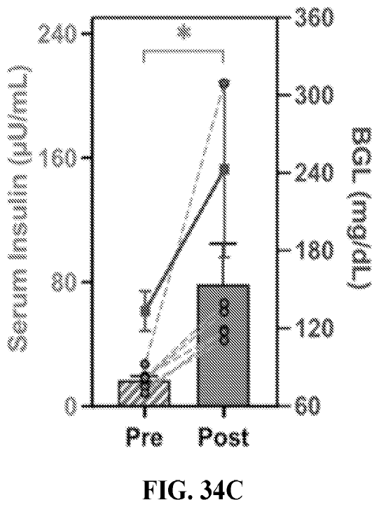

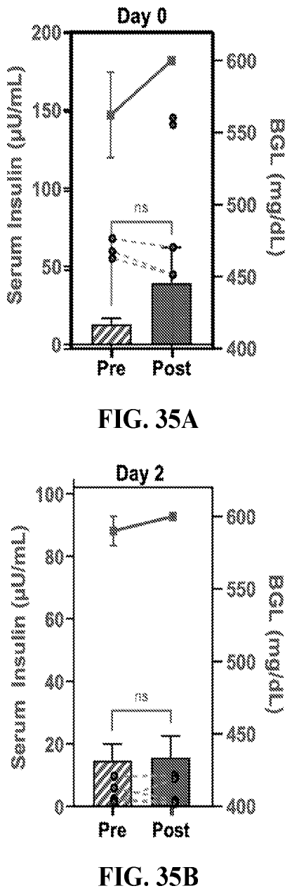

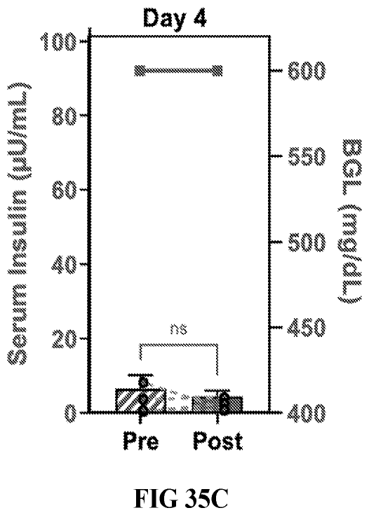

Multi-day blood glucose control from Insulin-Dendrimer formulations with sustained reduction and responsive correction when subjected to IPGTT at least supports protraction of insulin from the depot, beyond that observed by most long-lasting basal insulins; it also exceeds that reported from DiPBA based hydrogel delivery approaches, which only afforded blood glucose control for a single day. Verifying authentic glucose-responsive depot function—and not just ultra-long-lasting insulin-controlled release functionality—required further study of serum insulin concentrations in response to blood glucose challenge. Accordingly, serum insulin levels were monitored at 0, 2, and 4 d following treatment with the 1:1 complex in conjunction with IPGTT (FIGS. 34A-34C). Serum was collected from mice along with blood glucose measurements at 30 min prior and then 30 min following IPGTT. The presence of serum insulin prior to IPGTT on all 4 days supports continuous basal insulin availability from the depot, aligning with expectations for some level of release even under low glucose conditions. However, at day 0, 2, and 4 following treatment, serum insulin levels were elevated 30 min following IPGTT. The increase in serum insulin concentration at day 0 (+160%), 2 (+290%), and 4 (460%) amounted to an elevation over pre-challenge levels on all days. The general trend was strengthened by pre- and post-IPGTT serum insulin measurements having been taken from the same mouse. STZ mice administered the Dendrimer-Diol carrier alone were verified to have serum insulin levels at or below the limits of ELISA detection both before and after IPGTT (FIGS. 35A-35C). This result assures that ELISA-detected insulin was arising from the depot and not due to a glucose response emanating from residual pancreatic function that may result were STZ not effective at ablating the pancreatic § cells.

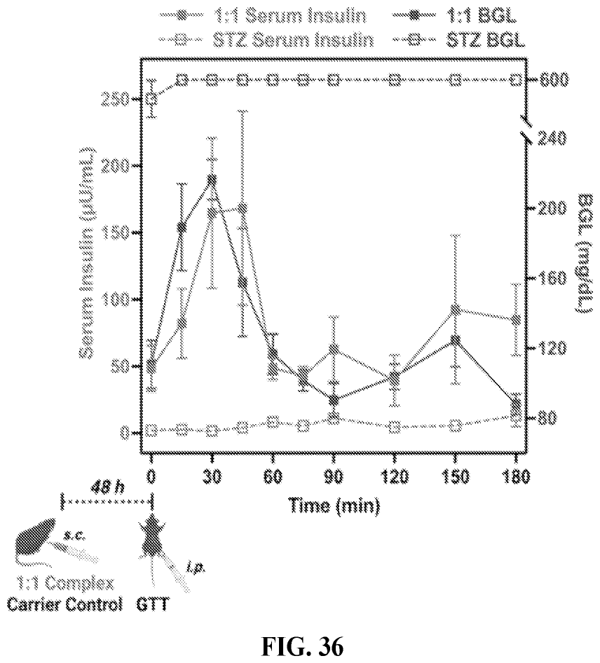

To further assess the kinetics of insulin availability from the depot, serum insulin levels were next measured serially in cohorts of mice administered the 1:1 nanocomplex during an IPGTT conducted 48 h after treatment (FIG. 36). Cohorts of mice all identically treated were necessary due to the blood volume collection requirements for accurate detection. At each timepoint, blood glucose was measured, and serum samples were collected. The general trend in blood glucose following challenge matched that seen for groups of mice tracked over the same time, verifying the validity of the cohort approach. Excitingly, even at 48 h after treatment with the 1:1 complex serum insulin was elevated with kinetics that corresponded to the increase in blood glucose from IPGTT and declined along with blood glucose correction. The difference between the average serum insulin concentration at the initial time (49 μU/mL) and at the peak (169 μU/mL) accounted for an increase of ~-340%; blood glucose levels rose from 110 mg/dL to 216 mg/dL for an increase of ~195% in this same time. STZ-induced mice administered only a carrier showed no increase in serum insulin upon IPGTT. These data support insulin bioavailability originating from the depot that directly corresponds to changes in blood glucose level. Such kinetics have not yet been reported in the literature of glucose-responsive insulin technologies to date, though spikes in serum insulin following glucose challenge have been shown.

### 2. Serial Dosing for Long-Term Blood Glucose Control

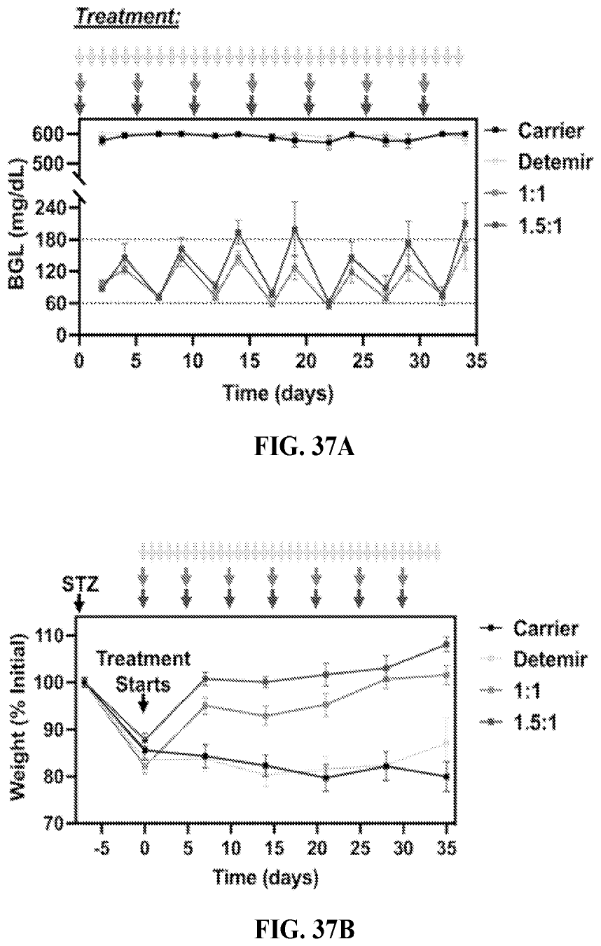

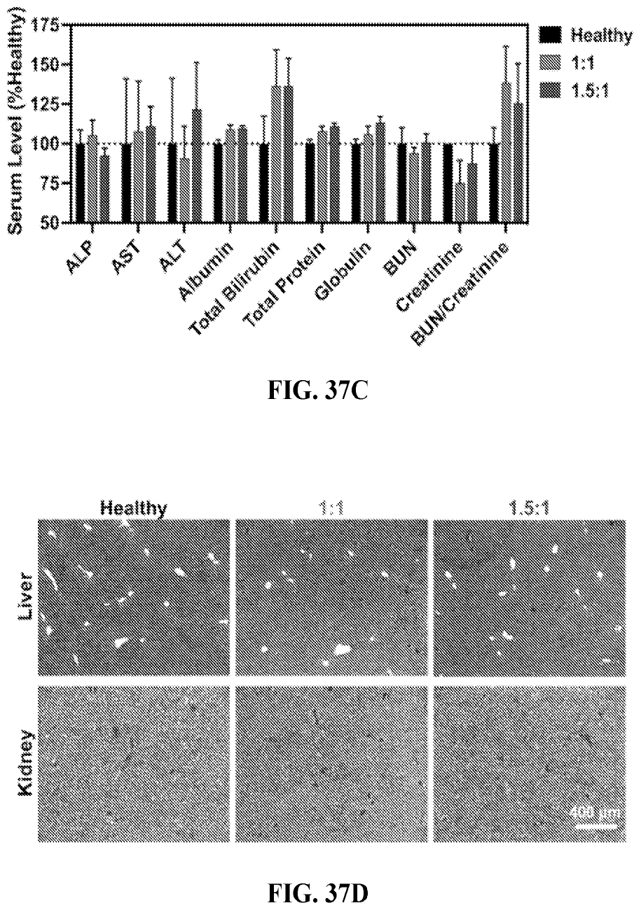

The function of Insulin-Dendrimer nanocomplexes was also explored in the context of repeat dosing over the course of 5 weeks, with complexes dosed every 5 d (FIG. 37A). At two and four days after administration of each dose, blood glucose was measured following a brief 3 h fast, included to correct for time since last meal and ensure gastric emptying. Using this dosing and assessment protocol, both the 1:1 and 1.5:1 complex resulted in mice with a normoglycemic range (60-180 mg/dL for healthy mice) throughout the study, with a comparative improvement for the time-in-range upon treatment with the 1:1 formulation, The same previously established potency-matched daily dose of Insulin Detemir, determined based on its ability to elicit a similar blood glucose correction in overnight-fasted mice, did not provide sustained blood glucose correction. The mice treated with Insulin-Dendrimer nanocomplexes also had improved body condition, better grooming, reduced polyuria, and were of noticeably better health status than either the carrier control or Insulin Detemir groups. Indeed, mice treated with the Insulin-Dendrimer nanocomplexes recovered to their pre-STZ body weights following only a week of treatment whereas the carrier control and the Insulin Detemir mice both maintained body weights ~20% reduced from their pre-STZ levels (FIG. 37B).

To further establish feasibility of serial redosing with the Insulin-Dendrimer nanocomplexes for long-term blood glucose control, these were dosed repeatedly in healthy mice for one month, after which serum chemistry was measured from samples collected at the study endpoint (FIG. 37C). These studies were performed in healthy mice to avoid confounding influence from the toxic effects of STZ on health status. Serum markers of liver and kidney function were selected for profiling, showing no noticeable differences observed for any of the markers relative to healthy mice treated with saline. Likewise, endpoint histology of liver and kidney tissue following serial dosing for one month revealed normal tissue structures with no histological abnormalities (FIG. 37D). These findings support limited impact from serial redosing of the Insulin-Dendrimer nanocomplexes on the metabolic function or overall health status in key organs associated with insulin signaling and clearance. In particular, liver inflammation has been a key barrier in the development of other synthetically modified insulins, and the lack of any inflammation here is therefore encouraging. It is envisioned that the Insulin-Dendrimer forms a nanoprecipitated depot following injection, like the mechanism of protraction for Insulin Glargine. A post-mortem search of the subcutaneous area following serial injection revealed no signs of inflammation or material accumulation, and thus the injection site (which varied slightly with every administration) was not able to be collected for analysis of local inflammation or material retention by histology.

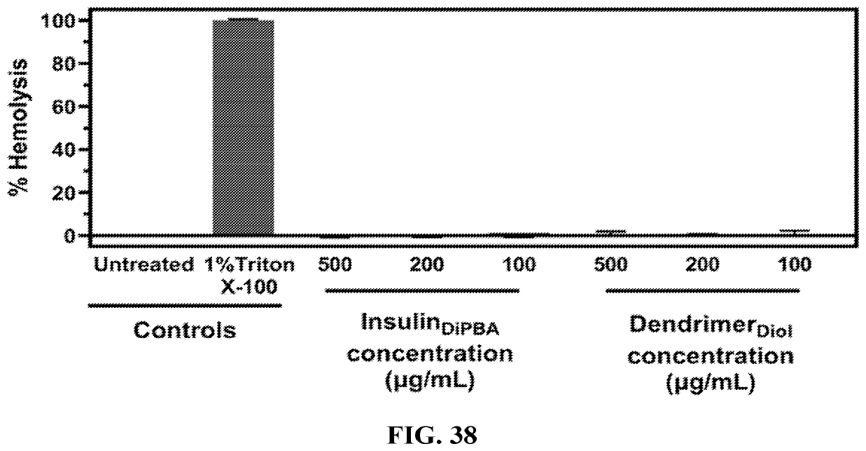

PAMAM dendrimers are known to have hemolytic properties, as their highly cationic surface charge may interact with and disrupt red blood cell membranes. This could present safety concerns in the context of translation of this current technology. A hemolysis assay was thus performed for Insulin-DiPBA and Dendrimer-Diol individually at concentrations ~1×-5× their maximum possible blood concentrations at the dosing levels used in mice (FIG. 38). No hemolysis was observed for either component in these studies. For the Dendrimer-Diol, this result is attributed to the highly reduced surface charge due to modification of ~80% of the terminal PAMAM amino groups with the diol.

### E. Example 4: Physiological Evaluation of Insulin-Dendrimer Nanocomplexes in Ossabaw Minipigs Blood Glucose Control in Ossabaw Minipigs

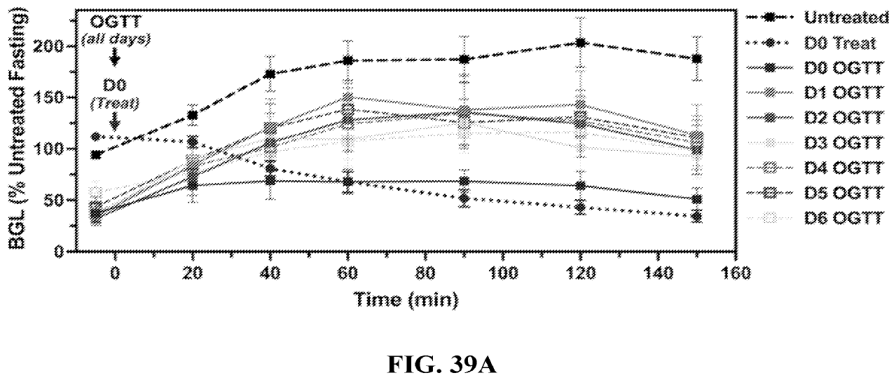

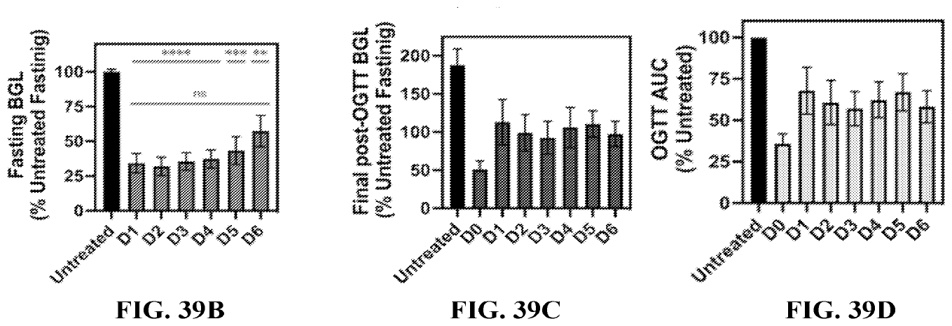

To assess the translational potential of the 1:1 Insulin-Dendrimer nanocomplex in a human-sized subject, a study was performed in alloxan-induced insulin deficient 11-14 month-old Ossabaw minipigs with body weights of ~41-59 kg (~90-130 pounds, n=6). Alloxan is commonly used in pigs to recreate insulin deficiency and hyperglycemia, pathological features of type 1 diabetes, due to its lower rate of mortality than STZ. Following alloxan treatment and blood glucose stabilization, a baseline oral glucose tolerance test (OGTT) administered via gavage was performed for two consecutive days on each pig in an overnight fasted state with no insulin treatment to determine the baseline untreated blood glucose response against which to compare each animal following treatment (FIG. 39A). The average untreated fasting blood glucose levels for all swine at the study outset was 228±43 mg/dL. Normal fasting blood glucose for healthy Ossabaw swine is in the range of roughly 57-71 mg/dL. As such, alloxan treatment successfully induced hyperglycemia reminiscent of an insulin deficient diabetic state. On the treatment day (D0), overnight-fasted swine were treated with the 1:1 Insulin-Dendrimer formulation at an insulin dose of ~-0.8-0.9 mg/kg; the exact dose was determined specifically for each animal using reported methods for allometric scaling of insulin dose to account for the difference in size and body surface area of mice and swine. Following treatment, blood glucose levels were reduced to ~35% of their initial fasted levels; after 3 h, the average blood glucose level for pigs was in the range of 71±12 mg/dL, within the range expected for healthy swine.

Three hours following treatment with the Insulin-Dendrimer formulation, a DO OGTT was performed (FIG. 39A). Subsequent rounds of OGTT were performed on overnight-fasted swine for 6 additional days (D1-D6) to evaluate the effectiveness of a single dose of the formulation for one week of blood glucose control. Blood glucose levels were generally reduced throughout the course of the week following treatment. This is most strongly evidenced by fasting blood glucose levels (FIG. 39B) that were ~32% of untreated fasting levels (73±15 mg/dL) in the early days following treatment. Though some elevation was seen over time, fasting blood glucose levels showed sustained control, even at D6 with levels that were ~57% of the untreated fasting blood glucose levels. The final blood glucose values for swine, collected at 150 min following OGTT, were also reduced to roughly half of their level in the untreated control state throughout D1-D6 following treatment (FIG. 39C). The blood glucose levels from the OGTT performed at DO were slightly depressed relative to subsequent days, likely due to increased insulin release following its initial administration. Dosing and injection volume could be further optimized in subsequent studies to limit the extent of initial burst release, though it is at the same time encouraging that swine did not exhibit signs of hypoglycemia resulting from administration of the Insulin-Dendrimer formulation. Overall, responsiveness as measured by the AUC throughout OGTT also was consistently reduced on D1-D6 following treatment compared to the untreated levels (FIG. 39D).

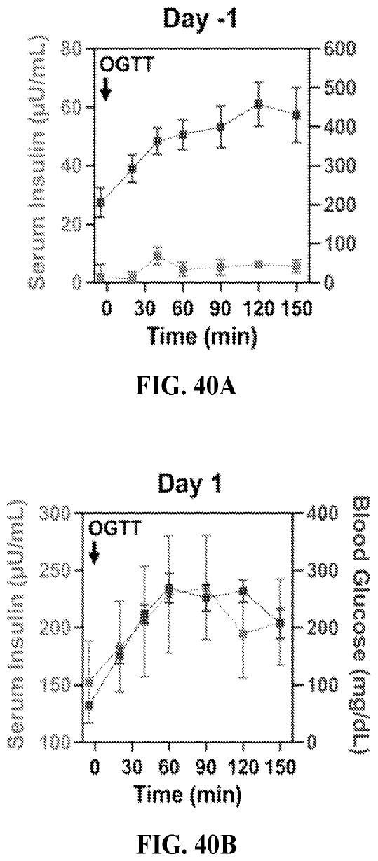

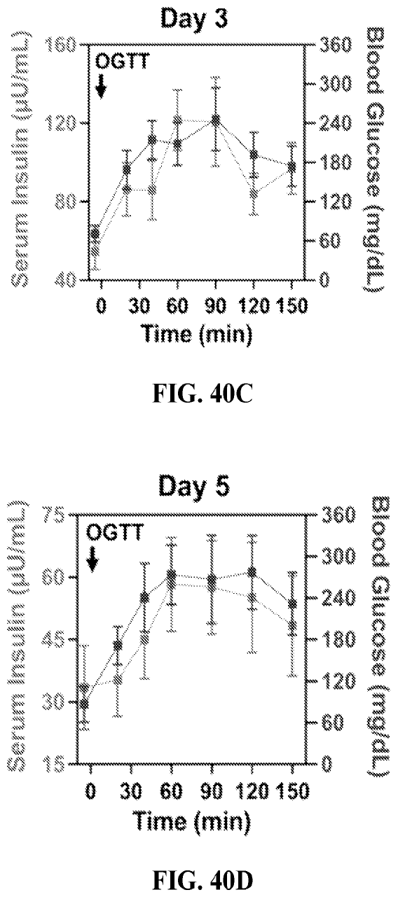

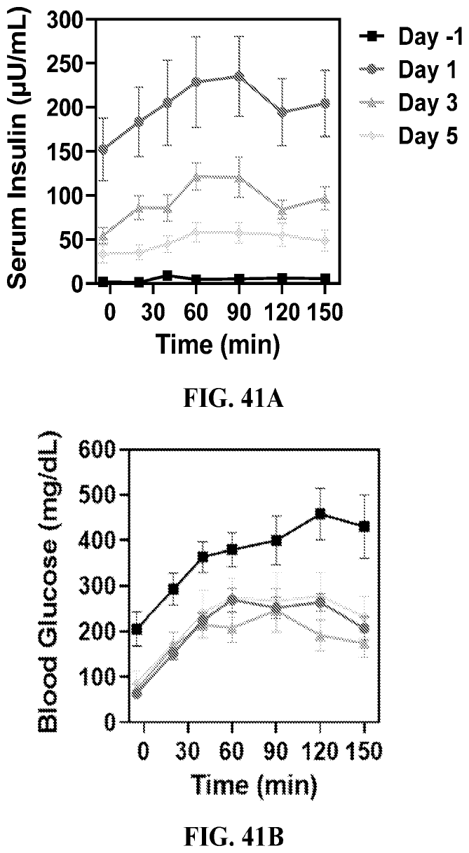

Serum insulin levels were furthermore quantified during OGTT on each day of the study (FIGS. 40A-40D). Prior to treatment with the Insulin-Dendrimer formulation (D-1), serum insulin levels were near or below the limits of ELISA detection and did not show any corresponding increase in response to OGTT. These data confirm effective loss of insulin-secreting function in alloxan-treated swine. Following treatment with the Insulin-Dendrimer formulation, serum insulin levels were quantifiable, and were correlated with blood glucose levels during the course of OGTT (FIGS. 40A-40D). As was observed in mouse studies, these data again point to glucose-triggered release of Insulin-DiPBA from the depot and increased serum insulin availability. Whereas the IPGTT in mouse studies indicated some lag in insulin levels following the increase in glucose, the serum insulin and blood glucose levels following OGTT in swine tracked more closely; this may be due to the comparatively slower rate of glucose absorption for oral versus intraperitoneal administration. It is noted that the serum insulin concentration released from the depot over the course of the OGTT decreased with time following treatment; these insulin levels were still effective in normalizing fasting blood glucose and controlling glucose levels throughout each OGTT cycle (FIGS. 41A-41B). Accordingly, in a human-sized animal model, the results support week-long blood glucose control and glucose-responsive function of this Insulin-Dendrimer nanocomplex formulation, both of which have never been reported at this scale.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:
Clause 1. A complex comprising:
   insulin attached to B¹ (insulin-B¹) and a macromolecule attached to D¹ (macromolecule-D¹); or
   insulin attached to D¹ (insulin-D¹) and a macromolecule attached to B¹ (macromolecule-B¹);
   wherein:
   B¹ is

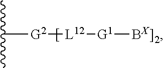

wherein:
B^{X}, at each occurrence, is B(OH)₂ or [B(OH)₃];
G¹, at each occurrence, is independently a pyridylene or a phenylene, wherein G¹ is optionally substituted with 1-2 substituents independently selected from the group consisting of halogen, —CN, C₁₋₄alkyl, —NO₂, C₃₋₄cycloalkyl, C₁₋₂haloalkyl, —OC₁₋₄alkyl, —OC₃₋₄cycloalkyl, and —OC₁₋₂haloalkyl, wherein each cycloalkyl is optionally substituted with 1-4 substituents independently selected from the group consisting of halogen, C₁₋₄alkyl, and C₁₋₂haloalkyl;
L¹², at each occurrence, is independently a C₁₋₆alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G2 is optionally substituted with a first substituent selected from the group consisting of halogen, cyano, C₁₋₆alkyl, C₁₋₄haloalkyl, oxo, —OR^{2x}, —N(R^{2x})₂, —SR^{2x}, —SO₂R^{2x}, —C(O)R^{2x}, —C(O)OR^{2x}, —C(O)N(R^{2x})₂, —C₁₋₆alkylene-OR^{2x}, —C₁₋₆alkylene-SR^{2x}, —C₁₋₆alkylene-N(R^{2x})₂, —C₁₋₆alkylene-SO₂R^{2x}, —C₁₋₆alkylene-C(O)R^{2x}, —C₁₋₆alkylene-C(O)OR^{2x}, and —C₁₋₆alkylene-C(O)N(R^{2x})₂, and optionally further substituted with 1-3 substituents independently selected from the group consisting of halogen, cyano, C₁₋₄alkyl, C₁₋₄haloalkyl, and —OR^{2x};
R^{2x}, at each occurrence, is hydrogen, C₃₋₄cycloalkyl, C₁₋₂haloalkyl, —OC₁₋₄alkyl, —OC₃-4cycloalkyl, or —OC₁₋₂haloalkyl, wherein each cycloalkyl is optionally substituted with 1-4 substituents independently selected from the group consisting of halogen, C₁₋₄alkyl, and C₁₋₂haloalkyl; and
D¹ is a diol moiety comprising

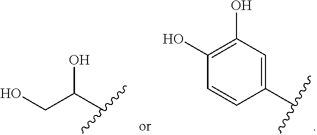 [SMILES: *C(O)CO ; *c1ccc(O)c(O)c1]

Clause 2. The complex of clause 1, wherein the complex comprises the insulin-B¹ and the macromolecule-D¹.
Clause 3. The complex of clause 2, wherein the molar ratio of the insulin-B¹ to the macromolecule-D¹ is from 1:1 to 110:1.
Clause 4. The complex of any one of clauses 1-3, wherein G² is phenylene.
Clause 5. The complex of any one of clauses 1-4, wherein

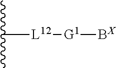

is:

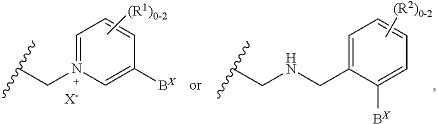

wherein:
R¹, at each occurrence, is independently halogen, —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl;
R², at each occurrence, is independently —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl; and
X⁻ is an anion having a net charge of −1.
Clause 6. The complex of clause 5, wherein X⁻ is Br⁻, Cl⁻, NO₃⁻, H₂PO₄⁻, H₂PO₃⁻, HSO₄⁻, HSO₃⁻, H₃C—SO₃⁻, HCO₃⁻, HCO₂⁻, H₃C—CO₂⁻, HC₂O₄⁻, or TsO⁻.
Clause 7. The complex of clause 6, wherein X⁻ is Br⁻ or Cl⁻.
Clause 8. The complex of any one of clauses 1-7, wherein B¹ is attached to the insulin by a linking moiety.
Clause 9. The complex of clause 8, wherein the linking moiety comprises:

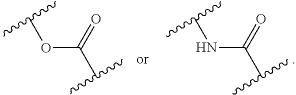 [SMILES: *OC(*)=O ; *NC(*)=O]

Clause 10. The complex of clause 9, wherein the linking moiety comprises:

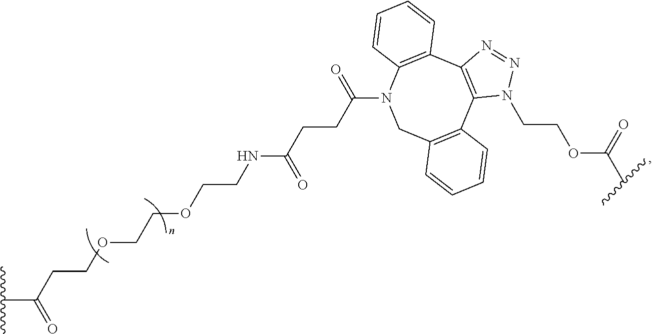

wherein n is 1 to 20.
Clause 11. The complex of any one of clauses 1-10, wherein B¹ is attached to the insulin's Lys_{B29} residue.
Clause 12. The complex of any one of clauses 1-11, wherein D¹ is:

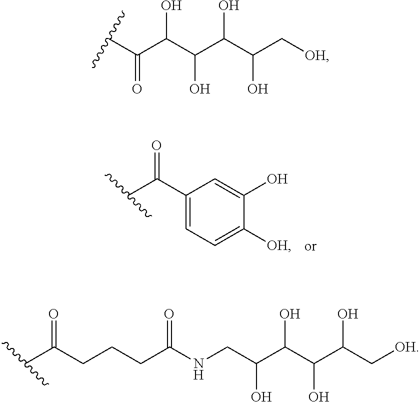 [SMILES: *C(=O)C(O)C(O)C(O)C(O)CO ; *C(=O)c1ccc(O)c(O)c1 ; *C(=O)CCCC(=O)NCC(O)C(O)C(O)C(O)CO]

Clause 13. The complex of any one of clauses 1-12, wherein the macromolecule is a dendrimer.
Clause 14. The complex of clause 13, wherein the dendrimer is a polyamidoamine dendrimer, a polyethylenimine dendrimer, a polyester dendrimer, or a lysine dendrimer.
Clause 15. The complex of clause 13 or 14, wherein the dendrimer is a 16-arm to 256-arm dendrimer.
Clause 16. A pharmaceutical composition comprising the complex of any one of clauses 1-15 and a pharmaceutically acceptable excipient.
Clause 17. A method of correcting blood glucose levels in a subject in need thereof, the method comprising: administering the complex of clause 1, or the pharmaceutical composition of clause 16, to a subject in need thereof.
Clause 18. The method of clause 17, wherein the subject in need thereof has diabetes.
Clause 19. The method of clause 17 or 18, wherein the insulin-B¹ is administered to the subject at 0.05 mg/kg to 10 mg/kg.
Clause 20. The method of any one of clauses 17-19, wherein following administration of the complex or pharmaceutical composition, the subject has blood glucose levels of 40 mg/dL to 800 mg/dL.
Clause 21. The method of any one of clauses 17-20, wherein following administration of the complex or pharmaceutical composition, the subject has corrected blood glucose levels for at least 1 day.
Clause 22. Use of the complex of any one of clauses 1-15 for delivering insulin to a subject in need thereof.

1. A complex comprising:
insulin attached to B¹ (insulin-B¹) and a macromolecule attached to D1 (macromolecule-D¹); or
insulin attached to D¹ (insulin-D¹) and a macromolecule attached to B¹ (macromolecule-B¹);
wherein:
B¹ is 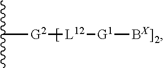
wherein:
B^{X}, at each occurrence, is B(OH)₂ or [B(OH)₃]—;
G¹, at each occurrence, is independently a pyridylene or a phenylene, wherein G¹ is optionally substituted with 1-2 substituents independently selected from the group consisting of halogen, —CN, C₁₋₄alkyl, —NO₂, C₃₋₄cycloalkyl, C₁₋₂haloalkyl, —OC₁₋₄alkyl, —OC₃₋₄cycloalkyl, and —OC₁₋₂haloalkyl, wherein each cycloalkyl is optionally substituted with 1-4 substituents independently selected from the group consisting of halogen, C₁₋₄alkyl, and C₁₋₂haloalkyl;
L¹², at each occurrence, is independently a C₁₋₆alkylene wherein optionally 1-2 methylene groups in the alkylene of L¹² are independently replaced with —N(H)—, —O—, or —S—, wherein 2 methylene groups replaced with —N(H)—, —O—, or —S— are separated by two or more carbon atoms in the alkylene; and
G² is phenylene, wherein G² is optionally substituted with a first substituent selected from the group consisting of halogen, cyano, C₁₋₆alkyl, C₁₋₄haloalkyl, oxo, —OR^{2x}, —N(R^{2x})₂, —SR^{2x}, —SO₂R^{2x}, —C(O)R^{2x}, —C(O)OR^{2x}, —C(O)N(R^{2x})₂, —C₁₋₆alkylene-OR^{2x}, —C₁₋₆alkylene-SR^{2x}, —C₁₋₆alkylene-N(R^{2x})₂, —C₁₋₆alkylene-SO₂R^{2x}, —C₁₋₆alkylene-C(O)R^{2x}, —C₁₋₆alkylene-C(O)OR^{2x}, and —C₁₋₆alkylene-C(O)N(R^{2x})₂, and optionally further substituted with 1-3 substituents independently selected from the group consisting of halogen, cyano, C₁₋₄alkyl, C₁₋₄haloalkyl, and —OR^{2x};
R^{2x}, at each occurrence, is hydrogen, C₃₋₄cycloalkyl, C₁₋₂haloalkyl, —OC₁₋₄alkyl, —OC₃₋₄cycloalkyl, or —OC₁₋₂haloalkyl, wherein each cycloalkyl is optionally substituted with 1-4 substituents independently selected from the group consisting of halogen, C₁₋₄alkyl, and C₁₋₂haloalkyl; and
D¹ is a diol moiety comprising

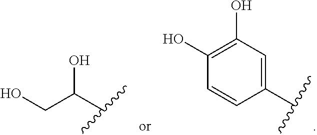 [SMILES: *C(O)CO ; *c1ccc(O)c(O)c1]

2. The complex of claim 1, wherein the complex comprises the insulin-B¹ and the macromolecule-D¹.

3. The complex of claim 2, wherein the molar ratio of the insulin-B¹ to the macromolecule-D¹ is from 1:1 to 110:1.

4. The complex of claim 1, wherein G² is phenylene.

5. The complex of claim 1, wherein 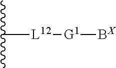
is: 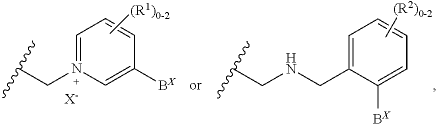
wherein:
R¹, at each occurrence, is independently halogen, —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl;
R², at each occurrence, is independently —CN, C₁₋₄alkyl, —NO₂, C₃₋₄cycloalkyl, C₁₋₂haloalkyl, —OC₁₋₄alkyl, —OC₃₋₄cycloalkyl, or —OC₁₋₂haloalkyl; and
X⁻ is an anion having a net charge of −1.

6. The complex of claim 5, wherein X⁻ is Br⁻, Cl⁻, NO₃⁻, H₂PO₄⁻, H₂PO₃⁻, HSO₄⁻, HSO₃⁻, H₃C—SO₃⁻, HCO₃⁻, HCO₂⁻, H₃C—CO₂⁻, HC₂O₄⁻, or TsO⁻.

7. The complex of claim 6, wherein X⁻ is Br⁻ or Cl⁻.

8. The complex of claim 1, wherein B¹ is attached to the insulin by a linking moiety.

9. The complex of claim 8, wherein the linking moiety comprises:

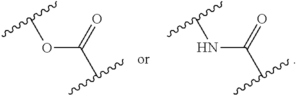 [SMILES: *OC(*)=O ; *NC(*)=O]

10. The complex of claim 9, wherein the linking moiety comprises: 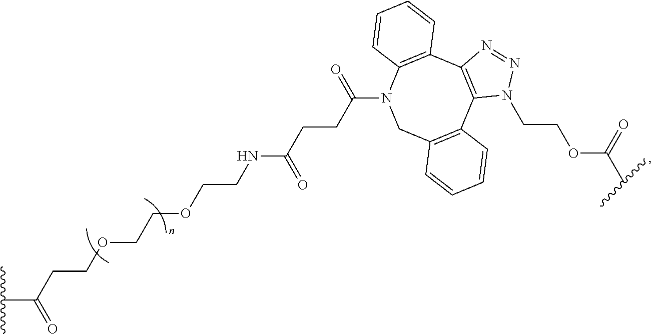
wherein n is 1 to 20.

11. The complex of claim 1, wherein B¹ is attached to the insulin's Lys_{B29} residue.

12. The complex of claim 1, wherein D¹ is:

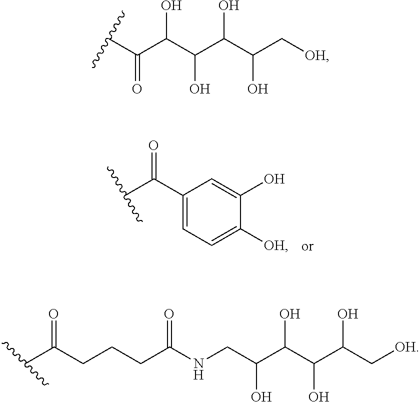 [SMILES: *C(=O)C(O)C(O)C(O)C(O)CO ; *C(=O)c1ccc(O)c(O)c1 ; *C(=O)CCCC(=O)NCC(O)C(O)C(O)C(O)CO]

13. The complex of claim 1, wherein the macromolecule is a dendrimer.

14. The complex of claim 13, wherein the dendrimer is a polyamidoamine dendrimer, a polyethylenimine dendrimer, a polyester dendrimer, or a lysine dendrimer.

15. The complex of claim 13, wherein the dendrimer is a 16-arm to 256-arm dendrimer.

16. A pharmaceutical composition comprising the complex of claim 1 and a pharmaceutically acceptable excipient.

17. A method of correcting blood glucose levels in a subject in need thereof, the method comprising: administering the complex of claim 1, or the pharmaceutical composition of claim 16, to a subject in need thereof.

18. The method of claim 17, wherein the subject in need thereof has diabetes.

19. The method of claim 17, wherein the insulin-B¹ is administered to the subject at 0.05 mg/kg to 10 mg/kg.

20. The method of claim 17, wherein following administration of the complex or pharmaceutical composition, the subject has blood glucose levels of 40 mg/dL to 800 mg/dL.

21. The method of claim 17, wherein following administration of the complex or pharmaceutical composition, the subject has corrected blood glucose levels for at least 1 day.

22. (canceled)
